(19) United States
(12) Patent Application Publication
(10) Pub. No.: US 2023/0365656 A1
(43) Pub. Date: Nov. 16, 2023

(54) A FUSION PROTEIN

(71) Applicant: THE UNIVERSITY OF QUEENSLAND, St. Lucia, Queensland (AU)

(72) Inventors: Mohammadmehdi MOBLI, Upper Mount Gravatt, Queensland (AU); Xinying JIA, Wishart, Queensland (AU); Yanni Ka-Yan CHIN, Sunnybank Hills, Queensland (AU); Alan Harry ZHANG, Canberra, Australian Capital Territory (AU); Theo Gene CRAWFORD, Toowong, Queensland (AU)

(21) Appl. No.: 18/009,224

(22) PCT Filed: Jun. 9, 2021
(86) PCT No.: PCT/AU2021/050578

§ 371 Date: Dec. 8, 2022

(30) Foreign Application Priority Data
Jun. 9, 2020 (AU) 2020901892

(51) Int. Cl. C07K 14/775; C12N 9/50

(52) U.S. Cl. CPC C07K 14/775; C12Y 304/22034; C12N 9/50; C07K 2319/50; C12Y 304/2207

(57) ABSTRACT

This invention relates to a method of producing a circularised form of a target protein from a fusion protein and to a fusion protein capable of producing a circularised form of a target protein. The fusion protein can comprise: the target protein; at least one circularisation site adjacent the target protein; and an enzyme domain capable of interacting with the at least one circularisation site and circularising the target protein. The target protein can be a membrane scaffold protein or a cyclotide such as SFTI, Vc1.1, Kalata B1 or MCOTI-II.

### CROSS REFERENCE TO RELATED APPLICATION

The present application is the U.S. national phase of International Application No. PCT/AU2021/050578 filed Jun. 9, 2021, which designated the U.S. and claims the benefit of and priority to Australian Provisional Application No. 2020901892, filed on Jun. 9, 2020, the entire disclosure of each of which is hereby incorporated by reference, in its entirety, for all that it teaches and for all purposes. This application includes an electronically submitted sequence listing in .txt format (0181_0451_Sequence_Listing_ST25.txt, 99,303 bytes). The sequence listing contained in this .txt file is part of the specification and is incorporated herein by reference in its entirety.

### TECHNICAL FIELD

This invention relates to fusion proteins. More particularly, this invention relates to “autocyclases” that comprise an enzyme domain capable of circularising a target protein, such as a membrane scaffold protein, cyclotide or a therapeutic protein, contained therein.

### BACKGROUND

Head-to-tail macrocyclisation is a naturally occurring post-translational modification¹ that endows peptides or proteins with various desirable properties, including stabilization of the protein fold often leading to enhanced proteolytic and thermal stability²⁻⁴. Thus, macrocyclisation has emerged as an attractive protein engineering tool. The most common strategy to achieve macrocyclisation is backbone cyclisation via a peptide bond. A widely used chemical method is native chemical ligation (NCL)⁵ for cyclising cysteine-containing peptides. NCL, however, requires an N-terminal cysteine and a C-terminal thioester. Furthermore, chemical peptide synthesis becomes a challenge for longer peptides and proteins (>100 amino acids). Consequently, several size-insensitive biological approaches have been developed, such as expressed protein ligation (EPL)⁶⁻⁹, split intein-mediated protein trans-splicing¹⁰, and genetic-code reprogramming¹¹. EPL and split-intein mediated protein circularization require a free cysteine in the sequence to perform an N—S acyl shift and trans-thioesterification, while backbone cyclisation via codon reprogramming requires the introduction of at least one nonproteinogenic amino acid.

In nature, peptides and proteins are cyclised by enzymes referred to as cyclases. Several native cyclases have been identified to date, these include the first characterized asparaginyl endopeptidase (AEP) butelase 1¹² and a related AEP called OaAEP1^{13,14} and three serine proteases PatG¹⁵, PCY1¹⁶, POPB¹⁷. The serine proteases are specific to the certain peptide sequences, spanning 5-11 residues whereas OaAEP1 and butelase 1 display broad substrate tolerance. The OaAEP1 C247A mutant possesses enhanced enzyme kinetics¹⁴, comparable to butelase 1. Both enzymes can be recombinantly expressed¹⁸, enriching the toolbox for peptide macrocyclisation.

Bioengineering efforts have approached cyclisation of proteins by repurposing ligases. These efforts have largely relied on the thiol-containing transpeptidases known as sortase A (SrtA) from Staphylococcus aureus¹⁹ for cyclisation of linear proteins²⁰. Examples include the disulfide-rich cyclotides²¹ and, more recently, the membrane scaffold proteins used in preparation of cyclised nanodiscs²².

Current peptide or protein cyclisation reactions require the addition of a polypeptide substrate to an enzyme in a bimolecular reaction. This approach, however, is very sensitive to substrate concentration and requires highly dilute reaction conditions to avoid polymerization of the substrate. Thus, the reaction mechanism, under standard conditions, never follows zero order enzyme kinetics but follows a bimolecular reaction mechanism and kinetics (where the substrate is very dilute and comparable in concentration to the enzyme). Current efforts of using ligases for circularisation of proteins are therefore prone to low yields and are not amenable to scale-up for industrial applications.

Accordingly, there remains a need for improved reagents and methods for circularising proteins or peptides that overcome one or more of the deficiencies of the prior art.

### DETAILED DESCRIPTION OF THE INVENTION

The present invention, in one or more embodiments, addresses a need to develop reagents and methods for the improved production speed and/or yield of circularised or cyclised proteins. In this regard, the present inventors have surprisingly discovered that in some embodiments fusion proteins designed to include a target protein and an enzyme domain capable of cyclising the target protein, upon recognition and binding of one or more flanking circularisation sites, are capable of advantageously producing cyclised/circularised versions of the target protein in higher yields, having lower impurities and with shorter production times than the prior art process of adding a polypeptide substrate to an enzyme in a bimolecular reaction. To this end, the present inventors have devised an engineered protein that self-cyclises/circularises, thus following a first order reaction mechanism that is independent of intermolecular collisions and therefore also invariant of diffusion limits.

In some embodiments, the present invention is predicated on the surprising discovery of a design for a fusion protein which facilitates the production or generation of a circularised or cyclic protein, such as a membrane scaffold protein, in higher yields and at higher concentrations and with lower impurities and shorter production times than methods of the prior art.

In one aspect, the invention relates to a fusion protein comprising a target protein flanked by at least one circularisation site and an enzyme domain that facilitates cyclising/circularising the target protein upon recognition and binding of the at least one circularisation site by the enzyme domain.

In another aspect, the invention relates to a fusion protein capable of producing a circularised form of a target protein, the fusion protein comprising:
the target protein;
at least one circularisation site adjacent the target protein; and
an enzyme domain capable of interacting with the at least one circularisation site and circularising the target protein.

The fusion protein, being a chimeric protein, may be of any suitable amino acid sequence and polymer length. The target protein, the at least one circularisation site and the enzyme domain are fused, connected or covalently bonded, typically into a single amino acid polymer. As described herein, the fusion protein can further comprise at least one spacer or linker, recognition motif, recognition site, affinity tag and/or domain. These too are fused, connected or covalently bonded, typically into a single amino acid polymer.

In some embodiments, the fusion protein can have an amino acid sequence or an encoding nucleotide sequence as shown in the Figures or Sequence Listing of this specification, or a fragment, variant, derivative or orthologue (ortholog) thereof.

It will be appreciated that the fusion protein may be provided in an isolated or purified form. For the purposes of this invention, by “isolated” or “purified” is meant material (such as a molecule) that has been removed from its natural state or otherwise been subjected to human manipulation. Isolated or purified material may be substantially or essentially free from components that normally accompany it in its natural state, or may be manipulated so as to be in an artificial state together with components that normally accompany it in its natural state. Isolated or purified proteins may be in native, chemical synthetic or recombinant form.

Any suitable type of target protein may be used. It may be of any suitable amino acid polymer length and amino acid sequence. It may comprise or consist of a naturally occurring amino acid polymer sequence. It may comprise or consist of a non-naturally occurring or engineered amino acid polymer sequence.

It is to be understood that a “target protein” includes within its scope a “protein”, “peptide” or “polypeptide”—namely, an amino acid polymer of any suitable length.

By “protein” is meant an amino acid polymer. The amino acids may be natural or non-natural amino acids, D- or L-amino acids as are well understood in the art. A “peptide” is a protein typically having less than fifty (50) amino acids. A “polypeptide” is a protein typically having fifty (50) or more amino acids. A “protein” can have less or more than fifty amino acids.

The terms “circularised”, “cyclised” and “cyclic” when used in reference to a protein refers to the fact that the amino acid sequence thereof is not linear in nature, such that it does not have an N-terminus or C-terminus. A protein which is circularised or cyclic can form any shape, such as a circle, an oval, or a polygon.

In some embodiments, the target protein can have an amino acid sequence or an encoding nucleotide sequence as shown in any one of the Figures or Sequence Listing of this specification, or a fragment, variant, derivative or orthologue (ortholog) thereof.

In some embodiments, the target protein is or comprises a membrane scaffold protein (MSP), or fragment, derivative or variant thereof. In some embodiments, when the membrane scaffold protein is circularised, it is suitably appropriate for use in the production of a nanodisc.

As used herein, the term “membrane scaffold protein” refers to a protein that can stabilize a phospholipid bilayer in a nanodisc by binding to the bilayer periphery thereof. In general, membrane scaffold proteins have hydrophobic faces that can associate with the nonpolar interior of a phospholipid bilayer and hydrophilic faces that favorably interact with a polar solvent such as an aqueous buffer. In some embodiments, the diameter of a circularised form of the membrane scaffold protein is between about 5 nm to about 80 nm (e.g., 5, 6, 7, 8, 9, 10, 11, 12, 13, 14, 15, 16, 17, 18, 19, 20, 25, 30, 35, 40, 45, 50, 55, 60, 65, 70, 75, 80 nm) and any range therein.

As used herein, “nanodisc” refers to a discoidal, nanoscale phospholipid bilayer which is “belted” or “ringed” by a membrane scaffold protein. The membrane scaffold protein generally comprises amphipathic alpha helices which provide a hydrophobic surface next to the hydrocarbon tails of the phospholipid bilayer and an external hydrophilic surface.

Nanodisc (ND) technology, and examples of membrane scaffold proteins, are known in the art and are described in, for example, U.S. Pat. Nos. 7,691,414; 7,662,410; 7,622,437; 7,592,008; 7,575,763; 7,083,958; and 7,048,949, each of which are hereby incorporated by reference in their entirety. In some embodiments, the membrane scaffold protein is that included or contained within an amino acid sequence set forth in any one of SEQ ID NOs:1 to 11, or a fragment, variant, derivative or orthologue (ortholog) thereof.

NDs typically comprise a nanometer-sized discoidal lipid bilayer wrapped by two copies of an α-helical MSPs. The initial MSPs were engineered forms of the major apolipoprotein ApoA1 in human high density lipoproteins (HDL).⁴¹ While a series of extended MSPs and deletion of N-terminal residues have been designed to incorporate membrane proteins and establish an optimal molar ratio of lipid to MSPs⁴², the helices in wild-type MSP1D1 have been further truncated to generate a panel of smaller NDs, including optimized MSP1D1ΔH5 for NDs suitable for solution structure determination of membrane proteins by NMR.^{43, 44} However, those MSPs are linear, resulting in the heterogeneity in the size of NDs and the number of membrane proteins assembled.⁴⁵ To improve the homogeneity of NDs, Nasr et al. have developed an evolved sortase A (eSrtA)-based method³⁰ to covalently join the N and C termini of MSPs to make circular MSPs (cMSPs) for circular NDs (cNDs).²²

In various embodiments described herein, a cyclic form of the target protein is for binding to a target of interest, which may be, for example, a therapeutic target or a pesticide target. The target of interest may be any molecule, including, but not limited to, a biomacromolecule such as a protein, a peptide, a nucleic acid (e.g., DNA or RNA), a polycarbohydrate, or a small molecule such as an organic compound or an organometallic complex, or any other molecule that contributes to a disease or is a target of a pesticide, such as the diseases listed below (e.g., a receptor for a therapeutic protein, an enzyme inhibited or activated by a therapeutic protein, or any other molecule wherein the activity of the molecule is altered by a therapeutic protein). In one embodiment, the target of interest can be a molecule involved in a disease state and the cyclic/circularised form of the target protein can be a therapeutic protein.

The disease that may be treated by the cyclic form of the target protein can be, for example, cancer, an infectious disease, heart disease (e.g., atherosclerosis) and other cholesterol-related diseases, stroke, wounds, pain, an inflammatory disease, such as arthritis (e.g., rheumatoid arthritis), inflammatory bowel disease, psoriasis, diabetes mellitus, or an autoimmune disease, a respiratory disease, such as asthma or chronic obstructive pulmonary disease, diarrheal diseases, a genetic disease, a neurological disorder, such as multiple sclerosis, Alzheimer's disease, muscular dystrophy, or Parkinson's disease, a mental disorder, or any other type of disease capable of being treated with a therapeutic protein (e.g., a cyclic form of the target protein). Particular examples of such target proteins are described in White and Craik, 2016 (Expert Opinion on Drug Discovery; vol. 11, pages 1151-1163), which is incorporated by reference in its entirety herein. Exemplary therapeutic proteins that are cyclic in nature include Sunflower trypsin inhibitor (SFTI), α-conotoxins (e.g., MII, Vc1.1, α-RgIA, α-ImI, α-AuIB, χ-MrIA, ω-MVIIA, PVIIA), Kalata B1, MCoTI-II, sea anemone peptide APETx2, hepcidin and gomesin, inclusive of orthologues, fragments, variants and derivatives thereof. Examples of fusion proteins comprising SFTI, Vc1.1 and Kalata B1 amino acid sequences and resultant circularised versions thereof are provided in FIGS. 4 and 16 and SEQ ID NOs:12 to 21, or orthologues, fragments, variants and derivatives thereof.

As used herein, the term “circularisation site” generally refers to a section, domain, motif or sequence of the fusion protein which upon interaction with the enzyme domain and optionally a corresponding further circularisation site in the fusion protein, can cause circularisation of the protein (i.e., the target protein). The circularisation site(s) can comprise one or more amino acid residues. A number of circularisation technologies are known in the art and any such technology can be applied to the fusion protein described herein. Exemplary circularisation domains are described below.

In various embodiments, the at least one circularisation site is a single circularisation site situated at, near, adjacent or next to the N- or C-terminal end of the target protein.

In various embodiments, the at least one circularisation site comprises first and second circularisation sites at, near, adjacent or next to respective N- and C-terminal ends of the target protein. In this regard, the arrangement of the first and second circularisation sites will at least partly depend upon the respective positions of the target protein and the enzyme domain (i.e., either N- or C-terminally) in the fusion protein.

In some embodiments, the first circularisation site is positioned at, near, adjacent or next to the N-terminal end of the target protein and the second circularisation site is positioned at, near, adjacent or next to the C-terminal end of the target protein. In such embodiments, the target protein is suitably positioned at, near, adjacent or towards an N-terminus of the fusion protein and the enzyme domain is suitably positioned at, near, adjacent or towards a C-terminus of the fusion protein.

In alternative embodiments, the first circularisation site is positioned at, near, adjacent or next to the C-terminal end of the target protein and the second circularisation site is positioned at, near, adjacent or next to the N-terminal end of the target protein. In such embodiments, the target protein is suitably positioned at, near, adjacent or towards a C-terminus of the fusion protein and the enzyme domain is suitably positioned at, near, adjacent or towards an N-terminus of the fusion protein.

Any suitable type of enzyme domain can be used. The term “enzyme domain” refers to a complete or partial amino acid sequence of at least one enzyme protein that includes the active or catalytic site thereof. To this end, the enzyme domain suitably includes an amino acid sequence of at least one enzyme or an enzymatically or catalytically active fragment, variant or derivative thereof.

As used herein an “enzyme” is a protein having catalytic activity towards one or more substrate molecules. Suitably, the enzyme is capable of displaying catalytic activity towards a substrate molecule (e.g., a linear protein) to thereby produce a cyclic/circularised protein by ligation of two segments of the target protein.

As generally used herein “enzymatically active”, “catalytically active”, “enzymatically active state” and “catalytically active state” may refer to absolute or relative amounts of enzyme activity that can be displayed or achieved by an enzyme or a fragment or portion thereof. Typically, an enzyme is enzymatically or catalytically active or in an enzymatically or catalytically active state if it is capable of displaying specific enzyme activity towards a substrate molecule to produce a cyclic peptide under appropriate reaction conditions. The enzyme domain can comprise, for example, at least one ligase and/or cyclase or an enzymatically active fragment, variant or derivative thereof.

In some embodiments, the enzyme domain can have an amino acid sequence or an encoding nucleotide sequence as shown in the Figures or Sequence Listing of this specification, or a fragment, variant, derivative or orthologue (ortholog) thereof. In some embodiments, the at least one circularisation site can have an amino acid sequence or an encoding nucleotide sequence as shown in the Figures or Sequence Listing of this specification.

In some embodiments, the ligase is or comprises a sortase or an enzymatically active fragment, variant or derivative thereof, such as Staphylococcus aureus wild type sortase A, evolved sortase (eSrtA), eSrtA(2A-9), eSrtA(4S-9), and Streptococcus pyogenes sortase A. Orthologues of these are also envisaged.

In particular embodiments, activity or activation of the sortase enzyme or enzymatically active fragments, variants or derivatives thereof is calcium dependent. Hence, activation of the sortase enzyme can comprise the step of adding calcium.

In some embodiments, the enzyme domain can be modulated by the introduction of a mutation or mutations as a result of structure-activity-relationship (SAR). In other embodiments, the enzyme domain can be modulated by the introduction of a mutation or mutations by directed evolution. In such embodiments, the enzyme domain can have an altered activity. The altered activity can result in: i) increased or reduced catalytic activity; ii) increased or reduced binding to the at least one circularisation site (ie. enzyme recognition site); or iii) an altered circularisation site (ie. recognition site). In particular, the unimolecular reaction design in these embodiments of the invention may create a kinetic gap between intra- and inter-molecular reactions, which in cases where the enzyme activity is reduced via the above changes (ie. mutations that reduce enzyme activity by any one of or any combination of i) to iii)) can ensure that the reaction can be carried out at relatively high substrate concentrations without a risk for polymerization, thus increasing overall yield and reaction efficiency. Mutations that reduce the enzyme activity by any one of or any combination of i) to iii) are known and are listed in Table 4. The mutation can be other than the motif LPXTG.

The use of sortase enzymes to circularise proteins is described in more detail in, for example, Cowper et al. ChemBioChem 2013 14:809-812; Antes et al., Journal of Biological Chemistry 2009 284: 16028-36; and Tsukiji et al. ChemBioChem 2009 10:787-798; each of which is incorporated by reference herein in its entirety. Sortase enzyme variants or derivatives are known in the art, such as those reviewed in Antos et al. (Curr Opin Struct Biol. 2016 June; 38: 111-118), which is incorporated by reference herein. In certain embodiments, a sortase enzyme variant includes a substitution of a cysteine residue in an active site thereof with, for example, a selenocysteine residue or a homocysteine residue.

According to such embodiments, a preferred at least one circularisation site is or comprises a sortase acceptor motif and/or a sortase recognition motif.

The term “sortase acceptor motif” refers to a moiety that acts as an acceptor for the sortase-mediated transfer of a polypeptide to the sortase acceptor motif. In particular embodiments, the sortase acceptor motif is located at, near, adjacent or towards the N- or C-terminus of the fusion protein and comprises a non-polar amino acid sequence, said sequence being at least one amino acid in length. In some embodiments, the non-polar amino acid sequence is from 1 to 20 amino acids (e.g., 1, 2, 3, 4, 5, 6, 7, 8, 9, 10, 11, 12, 13, 14, 15, 16, 17, 18, 19, 20 amino acids or any range therein) in length. In certain embodiments, the non-polar amino acid sequence is or comprises one or a plurality of glycines or alanines. Exemplary sortase acceptor motifs include Gly-[Gly]ₙ, wherein n=0-5 and Ala-[Ala]ₙ, wherein n=0-5. In particular embodiments, the first circularisation site is or comprises a sortase acceptor motif.

As generally used herein, the term “sortase recognition motif” as that term is used herein, refers to a polypeptide or peptide sequence which, upon cleavage by a sortase molecule, forms a thioester bond with the sortase molecule. In certain embodiments, the sortase recognition motif comprises the amino acid sequence LPXTG/A, LAXTG or LPXSG, where X is any amino acid. It will be appreciated that sortase cleaves the sequence after the threonine or serine. The threonine or serine residue can then form a new peptide bond with a glycine or alanine residue found at a second, more N- or C-terminal location in the fusion protein, causing the target protein to be circularised.

In this regard, it will be appreciated that sortase-mediated cleavage generally occurs between the T/S and G/A residues of the recognition motif. In an embodiment, the peptide bond between T/S and G/A is replaced with an ester bond to the sortase molecule. In some embodiments, the amino acid sequence of LPXTG/A is or comprises: LPGTG/A; LPSTG/A; or LPETG/A. More particularly, the amino acid sequence of LPXTG/A is suitably: LPGTG; LPGTA; LPSTG; LPSTA; LPETG; or LPETA. In particular embodiments, the second circularisation site is or comprises a sortase recognition motif.

Although the natural recognition motif SrtA is LPxTG (where X is any amino acid), it is possible to generate mutants of SrtA that have a different recognition motif using methods of molecular evolution. These enzymes would have altered kinetic properties and can be used to generate cyclic target protein products with fewer amino acids due to the ligation scar (LPXTG).

Reported examples of this approach include: eSrtA(4S-9) for LPXSG linking sequences; eSrtA(2A-9) for LAXTG; SrtA-F40 and SrtA-A1-22 for APXTG; SrtA-F1-20 for FPXTG; and SrtAβ for LMVGG (where X is any amino acid). Thus, embodiments of the invention include any recognition motif that can be engineered for ligation recognition by the enzyme domain of the fusion protein—including but not limited to the above examples.

In certain embodiments, the enzyme domain is or comprises an asparaginyl endopeptidase (AEP) enzyme, inclusive of enzymatically or catalytically active fragments, variants or derivatives thereof, that preferentially functions as a cyclase. Exemplary AEP enzymes that demonstrate cyclase activity include butelase-1 and O. affinis AEP (OaAEP) and orthologues thereof.

Accordingly, in some embodiments, the enzyme domain is or comprises a butelase enzyme, inclusive of enzymatically or catalytically active fragments, variants or derivatives thereof. It will be appreciated that butelase-1 recognises a tripeptide motif, Asx-His-Val, where Asx is Asn (N) or Asp (D), at the C-terminus of a target protein, and mediates peptide backbone cyclisation/circularisation by cleaving the sorting sequence His-Val and ligating the Asx residue to the N-terminal residue of the target protein to form a circular topology. The use of butelase enzymes to circularise proteins is described in more detail in, for example, WO2015163818, WO2017058114 and Hemu et al. (2019) Butelase 1-Mediated Ligation of Peptides and Proteins. In: Nuijens T., Schmidt M. (eds) Enzyme-Mediated Ligation Methods. Methods in Molecular Biology, vol 2012. Humana, New York, NY; which are incorporated by reference herein in their entirety.

According to such embodiments in which the enzyme domain of the fusion protein comprises a butelase enzyme, or an enzymatically active fragment, variant or derivative thereof, the at least one circularisation domain suitably comprises a butelase recognition motif, such as Asp-His-Val or Asn-His-Val, positioned at, near, adjacent, next to or towards the C-terminus of the amino acid sequence of the target protein.

In certain embodiments, the enzyme domain is or comprises an OaAEP enzyme, inclusive of enzymatically or catalytically active fragments, variants or derivatives thereof, that preferentially functions as a cyclase (e.g., OaAEP1). It will be appreciated that OaAEP1 generally recognises a tripeptide motif, Asn-Gly-Leu at the C-terminus of a target protein, and mediates peptide backbone cyclisation/circularisation by cleaving the sequence Gly-Leu and ligating the Asn residue to the N-terminal residue of the target protein to form a cyclic protein. The use of OaAEP enzymes, as well as variants, derivatives and orthologues thereof to circularise proteins is described in more detail in, for example, Harris et al., Nature Communications, 2015; 6: 10199 and WO2017049362; which are incorporated by reference herein in their entirety.

According to such embodiments in which the enzyme domain of the fusion protein comprises a OaAEP enzyme, or an enzymatically active fragment, variant or derivative thereof, the at least one circularisation site suitably comprises an AEP recognition motif, such as Asn-Gly-Leu (i.e., NGL), positioned at, near, adjacent or towards the C-terminus of the target protein.

In particular embodiments, the AEP recognition motif comprises the amino acid sequence of X₁X₂X₃, wherein X₁ is N or D; X₂ is G or S; and X₃ is L, A or I.

In some embodiments, the at least one circularisation site of the fusion protein further comprises an AEP acceptor motif. Suitably, the AEP acceptor motif is at, near, adjacent, next to or towards a C-terminus of the target protein and may comprise the amino acid sequence X₄X₅, wherein X₄ is optional and any amino acid or G, Q, K, V or L; and X₅ is optional or any amino acid or L, F or I or a hydrophobic amino acid residue. In particular embodiments, the AEP acceptor motif is or comprises the amino acid sequence GL. It will be appreciated, however, that the fusion protein may not necessarily comprise any specific AEP acceptor motif, as AEP enzymes have been shown to cyclise/circularise target proteins in their absence.

The fusion protein may further comprise at least one spacer. The at least one spacer may be of any suitable amino acid polymer length and amino acid sequence. As used herein, the term “spacer” (also referred to herein as “linker”) refers to an amino acid polymer between two protein moieties, portions, domains, modules, sites, motifs etc. of the fusion protein. Spacers are typically designed to have flexibility or to insert a structure between two protein moieties, portions, domains, modules, sites, motifs etc., such as an alpha helix. In some embodiments, the at least one spacer can have an amino acid sequence or an encoding nucleotide sequence as shown in the Figures or Sequence Listing of this specification.

In some embodiments, the at least one spacer may be situated between the target protein and the enzyme domain.

Illustrative examples of spacers include glycine polymers (G)ₙ where n is an integer of at least one, two, three, four, or five; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains (e.g., the enzyme domain and the target protein) of the fusion protein. The ordinarily skilled artisan will recognize that design of a fusion protein in particular embodiments can include spacers that are all or partially flexible, such that the spacer can include a flexible spacer as well as one or more portions that confer less flexible structure to provide for a desired fusion protein structure.

Suitably, the spacer is engineered, such as with respect to length and/or flexibility, so that the enzyme domain may co-localize with, recognise and bind to one or more of the circularisation sites in the same fusion protein without steric hindrance. Accordingly, the spacer may be of any appropriate length and flexibility so as to facilitate access or binding of the catalytic or active site of the enzyme domain to at least one of the circularisation sites/domains of the same fusion protein.

In particular embodiments, the spacer may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues in length, optionally between 1-5, 1-10, 1-15, 1-20, 1-25, 1-30, 5-10, 5-15, 5-30, 10-15, 10-20 or 10-30 amino acid residues in length, preferably between 5-20 amino acid residues in length. The spacer may be a G/S-rich linker, i.e., an amino acid sequence comprising at least 50%, 60%, 70%, 80%, 85%, 90%, 95% or about 100% glycine and serine residues. Exemplary spacer sequences include GGS, GS(GGS)ₙ, GAAA and LEGT. In particular embodiments, the spacer may be approximately 35 to 45 Angstroms (Å) in length, including all numerical values between 35 and 45, including 35, 36, 37, 38, 39, 40, 41, 42, 43, 44, and 45 A.

The fusion protein may further comprise at least one inhibitory domain or region (or protecting domain or region). The at least one inhibitory domain may be of any suitable amino acid polymer length and amino acid sequence. The fusion protein typically comprises one or two inhibitory domains. In some embodiments, the at least one inhibitory domain or region (including a recognition sequence) can have an amino acid sequence or an encoding nucleotide sequence as shown in the Figures or Sequence Listing of this specification.

In some embodiments, the at least one inhibitory domain is or comprises a cap sequence adjacent the at least one circularisation site. In this regard, the inhibitory domain is suitably configured to inhibit recognition of the at least one circularisation site by the enzyme domain. In certain embodiments, the at least one inhibitory domain is or comprises an enzyme inhibitory sequence configured to inhibit activity of the enzyme domain by contacting the enzyme domain directly. By way of example, the fusion protein can comprise an inhibitory domain positioned N-terminally to a first circularisation site positioned at, near, adjacent or towards an N-terminus of the fusion protein, such that cleavage of the inhibitory domain exposes the circularisation site as the N-terminus of the fusion protein and/or removes the enzyme inhibitory sequence from the fusion protein. Similarly, the fusion protein can comprise an inhibitory domain positioned C-terminally to a circularisation domain positioned at, near, adjacent or towards a C-terminus of the fusion protein, such that cleavage of the inhibitory domain exposes the circularisation domain as the C-terminus of the fusion protein and/or removes the enzyme inhibitory sequence from the fusion protein. Accordingly, the inhibitory domain is suitably capable of being removed or cleaved to release the enzyme inhibitory sequence and/or expose the at least one circularisation site adjacent thereto as the N- or C-terminus of the fusion protein.

In particular embodiments, the inhibitory domain is positioned adjacent a first circularisation site, such as in embodiments in which the first circularisation site comprises one or a plurality of glycine residues and is positioned N-terminally to the target protein.

Such inhibitory domain sequences can comprise and be cleaved by means of a protease cleavage sequence recognized by a respective protease. A “protease” is any protein which displays, or is capable of displaying, an ability to hydrolyse or otherwise cleave a peptide bond. Like terms include “proteinase” and “peptidase”. Proteases include serine proteases, cysteine proteases, metalloproteases, threonine proteases, aspartate proteases, glutamic acid proteases, acid proteases, neutral proteases, alkaline proteases, exoproteases, aminopeptidases and endopeptidases although without limitation thereto. Proteases may be purified or synthetic (e.g., recombinant synthetic) forms of naturally-occurring proteases or may be engineered or modified proteases which comprise one or more fragments or domains of naturally-occurring proteases which, optionally, have been further modified to possess one or more desired characteristics, activities or properties.

Proteases are found throughout nature, including in viruses, bacteria, yeasts, plants, invertebrate animals and vertebrates, inclusive of mammals and humans, although without limitation thereto. Accordingly, proteases are involved in a variety of different physiological processes including digestion of food proteins, blood-clotting cascades, the complement system, apoptosis pathways, the invertebrate prophenoloxidase-activating cascade, bacterial exotoxins and processing of viral proteins, although without limitation thereto.

A preferred class of proteases are derived from, or encoded by, a viral genome. Typically, such proteases are dependent on expression and proteolytic processing of a polyprotein and/or other events required as part of the life cycle of viruses such as Picomavirales, Nidovirales, Herpesvirales, Retroviruses and Adenoviruses, although without limitation thereto. Particular examples of proteases include: Potyviridae proteases such as the NIa protease of tobacco etch virus (TEV), tobacco vein mottling virus (TVMV), sugarcane mosaic virus (SMV) etc; Flaviviridae proteases such as the NS3 protease of hepatitis C virus (HCV); Picornaviridae proteases such as the 3C protease of EV71, Norovirus etc, the 2A protease of human rhinovirus, coxsackievirus B4 etc and the leader protease of foot and mouth disease virus (FMDV) etc; Coronaviridae proteases such as the 3C-like protease of SARS-CoV, IBV-CoV and Herpesvirus proteases such as HSV-1, HSV-2, HCMV and MCMV proteases etc, although without limitation thereto.

A further class of exemplary proteases include proteolytic coagulation factors. The term “proteolytic coagulation factor” is to be understood as any plasmatic serine protease which has a procoagulant, anticoagulant or fibrinolytic (clot-dissolving) function in the blood clotting system of a mammal, such as a human. Nonlimiting examples of proteolytic coagulation factors include factor IIa (thrombin), factor VIIa, factor IXa, factor Xa, factor XIa, factor XIIa, activated protein C and plasmin.

By way of example, the inhibitory domain can comprise the recognition sequence Leu-Val-Pro-Arg-Gly-Ser. This recognition sequence can be cleaved by thrombin between Arg and Gly. In other embodiments, the inhibitory domain can comprise the recognition sequence Glu-Asn-Leu-Tyr-Phe-Gln-(Gly/Ser) which is recognized by TEV protease and cleaves between the Gln and Gly/Ser residues.

In particular embodiments, the inhibitory domain comprises a first protease cleavage site cleavable by a first protease to expose the at least one circularisation sites adjacent thereto and release inhibition of recognition and binding of the at least one circularisation site by the enzyme domain. By virtue of this arrangement, the fusion protein can be switched or converted from an enzymatically inactive form or state to an enzymatically active form or state capable of circularising the target protein.

As mentioned, the fusion protein can include an inhibitory domain or region at the N-terminus that can be removed by a protease to expose the N-terminal glycine require for the cyclisation/circularisation reaction by the enzyme domain. This ensures that the “autocyclase” (ie. a fusion protein that comprise an enzyme domain capable of circularising a target protein) remains catalytically inactive during expression and purification. However, if the inhibitory domain is removed such that the second amino acid is a glycine, the protein will become reactive after expression in a bacterial host, as the first (initiating) amino acid Met is efficiently removed immediately after translation by endogenous Met aminopeptidase (MAP). The premature activation of the fusion protein in this method will result in some unwanted in vivo reaction; however, it does remove the need for downstream need of a protease for removal of the inhibitory domain. In cases where the losses due to premature reaction of some of the fusion protein can be tolerated, this presents a method for generating cyclic target proteins without the use of an externally added protease—potentially reducing overall costs/time of production. This strategy has been demonstrated for MSP9-autocyclase and MSP11-autocyclase.

The fusion protein may comprise at least one affinity/purification tag. The at least one affinity may be of any suitable amino acid polymer length and amino acid sequence. In some embodiments the fusion protein comprises an affinity tag at an N- and/or C-terminus thereof, and more particularly, a C-terminus thereof. It will be appreciated that affinity tags can be used for affinity purification or to bind a protein to a desired substrate or surface. Accordingly, the affinity tag may facilitate isolation or purification of fusion protein molecules, such as where protein translation has proceeded to the C-terminus of the fusion protein. Suitably, the affinity tag is adjacent the enzyme domain of the fusion protein. The affinity tag suitably comprises an amino acid sequence of an epitope tag, fusion partner or other moiety that facilitates isolation and purification of the recombinant fusion protein. Additionally, and as described in further detail herein, the affinity tag preferably enables isolation and purification of the enzyme domain once it has been removed from the fusion protein and/or the spacer and after formation of a cyclic form of the target protein thereby.

A number of affinity tags and their binding partner ligands are well known in the art and are described, e.g., in Lichty et al. Protein Expr Purif 2005 41:98-105; Zhao et al. J Analytical Methods in Chemistry 2013; Kimple et al. Current Protocols in Protein Science 2004; Giannone et al. Methods and Protocols “Protein Affinity Tags” Humana Press 2014; Kimple et al. Current Protocols in Protein Science “Overview of Affinity Tags for Protein Purification” 2013; 73: Unit-9.9, each of which is incorporated by reference herein in their entirety. Well known examples of fusion partners include, but are not limited to, glutathione-S-transferase (GST), maltose binding protein (MBP) and metal-binding moieties such as polyhistidine (e.g., HIS₆ and HIS₁₀), for which affinity purification reagents are well known and readily available. Epitope tags are usually short peptide sequences for which a specific antibody is available. Well-known examples of epitope tags for which specific monoclonal antibodies are readily available include c-myc, influenza virus haemagluttinin and FLAG tags.

In certain embodiments, the affinity tag is an N- or C-terminal polyhistidine tag, such as a C-terminal hexahistidine (HIS₆) or decahistidine (HIS₁₀) tag, and/or a glutathione-S-transferase (GST) tag.

In some embodiments, the fusion protein comprises a first affinity tag at, near, adjacent or towards the N-terminus of the fusion protein and a second affinity tag at, near, adjacent or towards the C-terminus of the fusion protein. In such embodiments, the first affinity tag is suitably different from the second affinity tag. In this regard, the present inventors have found that the combination of two different affinity tags positioned at each respective terminus of the fusion protein advantageously facilitates isolation of a highly pure fusion protein during purification thereof.

In various embodiments, the fusion protein further comprises a second protease cleavage site positioned between the at least one spacer and the enzyme domain, the second protease cleavage site cleavable by a second protease to facilitate removal of the enzyme domain from the fusion protein or spacer following circularisation of the target protein. In some embodiments, the second protease cleavage site may form at least part of the at least one spacer.

The second protease and the second protease cleavage site may be any as are known in the art, such as those hereinbefore described. Suitably, however, the second protease and the second protease cleavage site are different from that of the first protease and the first protease cleavage site respectively. Such an arrangement advantageously facilitates a separate and step wise removal of the inhibitory domain and the enzyme domain from a remainder of the fusion protein.

It is envisaged that the fusion protein and the molecular components thereof described herein may be, or comprise, contiguous amino acid sequences as are well understood in the art. Optionally, respective amino acid sequences (e.g., the enzyme domain, the target protein amino acid sequence, a least one circularisation site, inhibitory domains, spacers etc) may be discrete or separate amino acid sequences linked or connected by further spacer or linker sequences (e.g., amino acids, amino acid sequences, nucleotides, nucleotide sequences or other molecules) to optimize features or activities such as circularisation site binding, enzyme domain inhibition and activity and target protein circularisation, although without limitation thereto. Non-limiting examples of amino acid and nucleotide sequences inclusive of fusion proteins, circularisation site/s, enzyme domains, inhibitory domains, spacers and protease cleavage sites are provided in the BRIEF DESCRIPTION OF THE SEQUENCES, particularly any one of SEQ ID NOS:1-34.

While the terms “first” and “second” are used in the context of respective, separate or discrete molecular components of the fusion protein, such as circularisation site/s, proteases, and/or protease cleavage sites, it will be appreciated that these do not relate to any particular non-arbitrary ordering or designation that cannot be reversed. Accordingly, the structure and functional properties of the first protease or second protease disclosed herein could be those of a second protease or a first protease, respectively. Likewise, the structure and functional properties of a first circularisation site and a second circularisation site disclosed herein could be those of a second circularisation site and a first circularisation site, respectively. Similarly, the structure and functional properties of a first protease cleavage site and the second protease cleavage site disclosed herein could be those of a second protease cleavage site and a first protease cleavage site, respectively. It will also be appreciated that the fusion protein may further comprise one or more other, non-stated molecular components. In this context, a “component” or “molecular component’ is a discrete molecule that forms a separate part, portion or component of the fusion protein.

Preferably, the fusion protein integrates protein expression, purification and circularisation into one single molecule, which may dramatically facilitate the production process at any scale.

The target protein may be circularised by way of a unimolecular reaction and circularisation can be achieved at theoretically infinitely low concentrations without loss in reaction rate. Thus, the reaction may be insensitive to scale and produces improve yields of cyclic target protein products compared to known techniques.

In view of the forgoing, and in particular embodiments, the fusion protein comprises from N-terminus to C-terminus:
optionally an inhibitory domain comprising a first protease cleavage site;
optionally a first circularisation site, such as a sortase acceptor motif or an OaAEP acceptor motif;
a target protein;
a second circularisation site, such as a sortase recognition motif, an OaAEP recognition motif or a butelase recognition motif,
optionally a spacer;
optionally a second protease cleavage site;
an enzyme domain; and
optionally an affinity tag.

In certain embodiments, the fusion protein comprises an amino acid sequence set forth in any one of SEQ ID NOs:1 to 21 or in any one of FIGS. 1, 2, 4, 7 and 14-16, or a fragment, variant, derivative or orthologue thereof. In certain embodiments, the fusion protein is encoded by a nucleotide sequence set forth in any one of SEQ ID NOs:22 to 34 or in any one of FIGS. 7 and 14-16, or a fragment, variant, derivative or orthologue thereof.

To this end, it will be understood by the skilled artisan that the invention includes fusion proteins that are variants of the embodiments described herein, or which comprise variants of the constituent enzyme domain, circularisation site/s, protease cleavage site/s, spacer/s, inhibitory domain/s and/or affinity tag/s amino acid sequences disclosed herein. Typically, such variants have at least 80%, at least 85%, preferably at least 90%, 91%, 92%, 93%, 94% 95%, 96%, 97%, 98% or 99% sequence identity with any of the amino acid sequences disclosed herein, such as SEQ ID NOs:1-21 or portions thereof. By way of example only, conservative amino acid variations may be made without an appreciable or substantial change in function. For example, conservative amino acid substitutions may be tolerated where charge, hydrophilicity, hydrophobicity, side chain “bulk”, secondary and/or tertiary structure (e.g., helicity), target molecule binding, enzyme or fluorescence activity are substantially unaltered or are altered to a degree that does not appreciably or substantially compromise the function of the fusion protein. Variants of the invention are selected to be functional and so retain or substantially retain catalytic activity, or the ability to reconstitute or activate such catalytic activity when provided together with suitable further components of a fusion protein as described above.

The term “sequence identity” is used herein in its broadest sense to include the number of exact amino acid matches having regard to an appropriate alignment using a standard algorithm, having regard to the extent that sequences are identical over a window of comparison. Sequence identity may be determined using computer algorithms such as GAP, BESTFIT, FASTA and the BLAST family of programs as for example disclosed by Altschul et al., 1997, Nucl. Acids Res. 25 3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

Protein fragments may comprise up to 5%, 10%, 15%, 20%, 25%, 30%, 35%, 40%, 45%, 50%, 55%, 60%, 65%, 70%, 75%, preferably up to 80%, 85%, more preferably up to 90% or up to 95-99% of an amino acid sequence disclosed herein. In some embodiments, the protein fragment may comprise up to 5, 10, 20, 40, 50, 70, 80, 90, 100, 120, 150, 180 200, 220, 230. 250, 280, 300, 330, 350, 400, 450, 500 or 550 amino acids of an amino acid sequence disclosed herein, such as SEQ ID NOS:1-21.

Derivatives of the fusion protein disclosed herein are also provided. As used herein, “derivative” proteins or peptides have been altered, for example by conjugation or complexing with other chemical moieties, by post-translational modification (e.g., phosphorylation, ubiquitination, glycosylation), chemical modification (e.g., cross-linking, acetylation, biotinylation, oxidation or reduction and the like), conjugation with labels (e.g., fluorophores, enzymes, radioactive isotopes) and/or inclusion of additional amino acid sequences as would be understood in the art. In this regard, the skilled person is referred to Chapter 15 of CURRENT PROTOCOLS IN PROTEIN SCIENCE, Eds. Coligan et al. (John Wiley & Sons NY 1995-2015) for more extensive methodology relating to chemical modification of proteins.

The fusion proteins, fragments, variants and/or derivatives of the present invention may be produced by any means known in the art, including but not limited to, chemical synthesis and recombinant DNA technology, such as hereinafter described.

Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2014). In this regard, reference is also made to International Publication WO 99/02550 and International Publication WO 97/45444.

Recombinant proteins may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2014), in particular Chapters 10 and 16; and CURRENT PROTOCOLS IN PROTEIN SCIENCE Eds. Coligan et al., (John Wiley & Sons, Inc. NY USA 1995-2014), in particular Chapters 1, 5 and 6.

In another aspect, the invention provides a method for circularising a target protein, said method including the steps of:
(a) providing a fusion protein comprising:
   a target protein;
   at least one circularisation site adjacent the target protein;
   an enzyme domain capable of circularising the target protein; and optionally a spacer positioned between the target protein and the enzyme domain;
(b) facilitating interaction of the enzyme domain with the at least one circularisation site to thereby circularise the amino acid sequence of the target protein.

Of course, the fusion protein may have one or more features as herein described in this specification.

In some embodiments, the present method further includes the initial step of producing the fusion protein prior to circularisation of the target protein. Such production may occur by any means known in the art, such as those hereinbefore described and including chemical and recombinant synthesis.

In various embodiments, the step of facilitating interaction, such as recognition, co-localisation, binding, cleavage and ligation thereby, of the enzyme domain with the at least one circularisation site comprises the step of activating the enzyme domain. In this regard, it will be appreciated that certain enzymes are only catalytically active under particular reaction conditions. By way of example, the activity of Sortase A can be calcium dependent. Accordingly, step (b) above is suitably performed under suitable conditions, such as in the presence of calcium ions and/or a detergent, appropriate or required for catalytic activity of the enzyme domain.

Suitably, the present method is performed under suitable ligation conditions. In particular embodiments, the present method is substantially performed in a suitable buffer or reaction mixture, such as a reaction buffer or ligation buffer. One of ordinary skill in the art will be familiar with a variety of buffers or reaction mixtures that could be used in accordance with the present invention. In some embodiments, the buffer solution or reaction mixture comprises calcium ions.

In certain embodiments, the buffer solution or reaction mixture does not contain substances that precipitate calcium ions. In some embodiments, the buffer solution or reaction mixture does not include phosphate ions. In various embodiments, the buffer solution or reaction mixture does not contain chelating agents. In particular embodiments, the buffer solution or reaction mixture comprises a detergent. In particular embodiments, the buffer solution or reaction mixture comprises a reagent to increase viscosity or a thickening agent, such as glycerol or glucose.

Accordingly, in some embodiments, the step of facilitating interaction of the enzyme domain with the at least one circularisation site is performed in a buffer or reaction mixture comprising about 0.5 mM to about 100 mM (e.g., 0.5, 1, 2, 3, 4, 5, 6, 7, 8, 9, 10, 15, 20, 25, 30, 35, 40, 45, 50, 60, 70, 80, 90, 100 mM and any range therein) of a calcium salt, such as calcium chloride.

In certain embodiments, the step of facilitating interaction of the enzyme domain with the at least one circularisation site is performed in a buffer or reaction mixture comprising about 0.1 mM to about 20 mM (e.g., 0.1, 0.2, 0.3, 0.4, 0.5, 1, 2, 3, 4, 5, 6, 7, 8, 9, 10, 11, 12, 13, 14, 15, 16, 17, 18, 19, 20 mM and any range therein) of a detergent, such as an alkyl maltoside like n-Dodecyl β-D-maltoside (DDM) or a non-ionic detergent like 2-[4-(2,4,4-trimethylpentan-2-yl)phenoxy]ethanol (i.e., Triton X-100). In alternative embodiments, the step of facilitating interaction of the enzyme domain with the at least one circularisation site is performed in a buffer or reaction mixture that is substantially free of a detergent (e.g., has no more than about 0.05 mM, 0.02 mM, 0.01 mM or 0.005 mM detergent).

In particular embodiments, the fusion protein is present, such as in the buffer or a reaction mixture, in a concentration of up to about 500 μM (e.g., about 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 475, 500 μM and any range therein). More particularly, the fusion protein can be present in a concentration from about 20 μM to about 200 μM, about 30 μM to about 150 μM or about 50 μM to about 100 μM.

In some embodiments, the present method is performed at least in part, such as the step of facilitating interaction of the enzyme domain with the at least one circularisation site (i.e., circularising the target protein), at a temperature of from about 0° C. to about 42° C. (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42° C. and any range therein) and more particularly, from about 0° C. to about 37° C. or about 20° C. to about 37° C.

In other embodiments, the present method is performed at least in part, such as the step of facilitating interaction of the enzyme domain with the at least one circularisation site (i.e., circularising the target protein), for a time from about 0.5 hours to about 48 hours (e.g., 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours and any range therein) and more particularly, from about 1 hour to 12 hours.

In some embodiments, the step of facilitating interaction of the enzyme domain with the at least one circularisation site comprises the step of removing or cleaving an inhibitory domain adjacent one or more of the at least one circularisation site. Once the inhibitory domain is removed, the enzyme domain and the at least one circularisation site are suitably capable of directly interacting (e.g., binding, coupling, co-localising and/or forming a complex therewith for subsequent cleavage and ligation thereof) to thereby form a circularised or cyclised version of the target protein. In particular embodiments, the step of removing the inhibitory domain comprises contacting the fusion protein with a first protease to cleave a first protease cleavage site of the inhibitory domain. It will be appreciated that the inhibitory domain, the first protease and the first protease cleavage site may be that as hereinbefore described.

Based on the foregoing, it will be appreciated that the present method may further include the subsequent step of removing the spacer from the enzyme domain of the fusion protein. In certain embodiments, the step of removing the spacer from the enzyme domain comprises contacting the fusion protein with a second protease to cleave a second protease cleavage site positioned between the spacer and the enzyme domain. It is envisaged that the second protease and the second protease cleavage site may be that as hereinbefore described.

In some embodiments, the present method further includes the subsequent step of isolating or purifying the enzyme domain removed from a remainder of the fusion protein by way of an affinity/purification tag positioned at, near, adjacent or towards an N- or C-terminus of the fusion protein and adjacent the enzyme domain. Suitably, the affinity tag enables isolation and purification of the enzyme domain, such as by binding to a desired substrate or surface, once it has been removed from the fusion protein and/or the spacer and after formation of a cyclic form of the target protein thereby.

It is envisaged that the method of the present aspect may further include the step of isolating or purifying the circularised target protein, such as from the reaction mixture or buffer as described herein.

In another aspect, the invention provides a circularised target protein, such as a membrane scaffold protein, produced according to the method of an aforementioned aspect.

In yet a further aspect, the invention provides an enzyme domain produced according to the method of an aforementioned aspect.

The skilled person will understand that the fusion proteins described herein can be utilised to produce circularised proteins, and more particularly circularised membrane scaffold proteins, which can be combined with phospholipids to form a covalently circularised nanodisc.

Accordingly, in still another aspect, the invention relates to a method of producing a nanodisc, said method including the steps of:
(a) providing a fusion protein comprising:
   a membrane scaffold protein;
   at least one circularisation site adjacent the membrane scaffold protein;
   an enzyme domain capable of circularising the membrane scaffold protein; and
   optionally a spacer positioned between the membrane scaffold protein and the enzyme domain;
(b) optionally facilitating interaction of the enzyme domain with the at least one circularisation site to thereby circularise the membrane scaffold protein; and
(c) contacting the fusion protein of (a) or the circularised membrane scaffold protein of step (b) with a lipophilic molecule(s) to thereby produce the nanodisc.

With respect to step (b), it will be appreciated that circularisation of the membrane scaffold protein may be performed substantially prior to, concurrently with and/or after step (c). Accordingly, in embodiments in which the fusion protein of (a) has been contacted with the lipophilic molecules, the method may include the step of facilitating interaction of the enzyme domain with the at least one circularisation site to thereby circularise the membrane scaffold protein.

Suitably, the fusion protein and/or the membrane scaffold protein are that previously described herein.

Suitably, the step of facilitating interaction of the enzyme domain with the at least one circularisation site and circularisation of the membrane scaffold protein is performed as hereinbefore described.

In this regard, the present method may further include the steps of:
activating the enzyme domain;
removing or cleaving an inhibitory domain adjacent one or more of the at least one circularisation site;
removing the spacer from the enzyme domain of the fusion protein; and/or
isolating or purifying the enzyme domain removed from the fusion protein by way of an affinity tag positioned at or towards an N- or C-terminus of the fusion protein and adjacent the enzyme domain,
such as by those means or methods previously described herein.

In certain embodiments, the lipophilic molecule is or comprises phospholipids, cholesterols, sphingomyelin, gangliosides, lipopolysaccharides, and derivatives of the foregoing.

Suitably, the lipophilic molecule, such as a solubilised lipophilic molecule, is or comprises a phospholipid, such as a solubilised phospholipid. As used herein, the term “phospholipid” refers to phosphatidic acids, phosphoglycerides, and phosphosphingolipids. Phosphatidic acids include a phosphate group coupled to a glycerol group, which may be mono- or di-acylated.

Phosphoglycerides (or glycerophospholipids) include a phosphate group intermediate an organic group (e.g., choline, ethanolamine, serine, inositol) and a glycerol group, which may be mono- or diacylated. Phosphosphingolipids (or sphingomyelins) include a phosphate group intermediate an organic group (e.g., choline, ethanolamine) and a sphingosine (non-acylated) or ceramide (acylated) group. It will be appreciated that in certain embodiments, the phospholipids useful in the compositions and methods of the invention include their salts (e.g., sodium, ammonium). For phospholipids that include carbon-carbon double bonds, individual geometrical isomers (cis, trans) and mixtures of isomers are included.

Representative phospholipids include phosphatidylcholines, phosphatidylethanolamines, phosphatidylglycerols, phosphatidylserines, phosphatidylinositols, and phosphatidic acids, and their lysophosphatidyl (e.g., lysophosphatidylcholines and lysophosphatidylethanolamine) and diacyl phospholipid (e.g., diacylphosphatidylcholines, diacylphosphatidylethanolamines, diacylphosphatidylglycerols, diacylphosphatidylserines, diacylphosphatidylinositols, and diacylphosphatidic acids) counterparts.

In some embodiments of any of the aspects, the method of the present aspect can comprise contacting the circularised membrane scaffold protein with one or a plurality of types of lipophilic molecule (e.g., 1, 2, 3, 4, 5 etc types of lipophilic molecules).

In some embodiments, the lipophilic molecules, such as phospholipids, described herein can further comprise a molecule of interest, such as a membrane protein, a receptor, a transmembrane protein or channel, hydrophobic small molecules, hydrophobic drugs, RNA, peptides, and the like. In some embodiments of any of the aspects described herein, the covalently circularised nanodiscs described herein can be or be utilized as a drug delivery vehicle.

In particular embodiments, the lipophilic molecules, such as phospholipids, are solubilized at least in part in a detergent. Exemplary detergents can include, but are not limited to Decylβ-D-maltopyranoside, Deoxycholic acid, Digitonin, n-Dodecyl β-D-glucopyranoside, n-Dodecyl β-D-maltoside, N-Lauroylsarcosine sodium salt, Sodium cholate, Sodium deoxycholate, Undecyl β-D-maltoside, Triton X-100, CHAPS, 5-Cyclohexylpentyl β-D-maltoside, n-dodecyl phosphatidylcholine, n-octyl-β-D-glucoside, and Brij 97.

In a related aspect, the invention resides in a nanodisc produced according to the aforementioned aspect.

In yet another aspect, the invention resides in a nucleic acid encoding the fusion protein described herein. It will be appreciated that the nucleic acid may be provided in an isolated or purified form. For the purposes of this invention, by “isolated” or “purified” is meant material (such as a molecule) that has been removed from its natural state or otherwise been subjected to human manipulation. Isolated or purified material may be substantially or essentially free from components that normally accompany it in its natural state, or may be manipulated so as to be in an artificial state together with components that normally accompany it in its natural state. Isolated or purified nucleic acids may be in native, chemical synthetic or recombinant form.

The term “nucleic acid” as used herein designates single- or double-stranded mRNA, RNA, cRNA, RNAi, siRNA and DNA inclusive of cDNA, mitochondrial DNA (mtDNA) and genomic DNA.

A “polynucleotide” is a nucleic acid having eighty (80) or more contiguous nucleotides, while an “oligonucleotide” has less than eighty (80) contiguous nucleotides. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “probe” may be a single or double-stranded oligonucleotide or polynucleotide, suitably labelled for the purpose of detecting complementary sequences in Northern or Southern blotting, for example.

In some embodiments, the isolated or purified nucleic acid encodes the fusion protein comprising the amino acid sequence set forth in any one of SEQ ID NOs:1-21 or in any one of FIGS. 1, 2, 4, 7 and 14-16, or a fragment, derivative, variant or orthologue thereof.

In various embodiments, the isolated or purified nucleic acid is that set forth in any one of SEQ ID NOs:22-34 or a fragment, derivative, variant or orthologue thereof. In certain embodiments, the nucleotide sequence is that set forth in any one of FIGS. 7 and 14-16, or a fragment, variant, derivative or orthologue thereof.

Also contemplated are fragments and variants of the isolated or purified nucleic acid. The invention also provides variants and/or fragments of the isolated nucleic acids. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence encoding the fusion protein of the invention (e.g., SEQ ID NOs:22-34).

Fragments may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence disclosed herein. Fragments may comprise or consist of up to 20, 50, 100, 150, 200, 250, 300, 350, 400, 450, 500, 550, 600, 650, 700, 750, 800, 850, 900 950, 1000, 1050, 1100, 1150, 1200, 1250, 1300, 1350, 1400, 1450, 1500, 1550, 1600, 1650 or 1700 contiguous nucleotides present in any nucleotide sequence disclosed herein.

The present invention also contemplates nucleic acids that have been modified such as by taking advantage of codon sequence redundancy. In a more particular example, codon usage may be modified to optimize expression of a nucleic acid in a particular organism or cell type.

The invention further provides use of modified purines (for example, inosine, methylinosine and methyladenosine) and modified pyrimidines (for example, thiouridine and methylcytosine) in isolated nucleic acids of the invention.

It will be well appreciated by a person of skill in the art that the isolated or purified nucleic acids of the invention can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008).

In yet another embodiment, complementary nucleic acids hybridise to nucleic acids of the invention under high stringency conditions. “Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Stringent conditions are well-known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Complementary nucleotide sequences may be identified by blotting techniques that include a step whereby nucleotides are immobilized on a matrix (preferably a synthetic membrane such as nitrocellulose), a hybridization step, and a detection step, typically using a labelled probe or other complementary nucleic acid. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20. According to such methods, Southern blotting involves separating DNA molecules according to size by gel electrophoresis, transferring the size-separated DNA to a synthetic membrane, and hybridizing the membrane bound DNA to a complementary nucleotide sequence. An alternative blotting step is used when identifying complementary nucleic acids in a cDNA or genomic DNA library, such as through the process of plaque or colony hybridization. Other typical examples of this procedure are described in Chapters 8-12 of Sambrook et al., MOLECULAR CLONING. A Laboratory Manual (Cold Spring Harbor Press, 1989).

Methods for detecting labelled nucleic acids hybridized to an immobilized nucleic acid are well known to practitioners in the art. Such methods include autoradiography, chemiluminescent, fluorescent and colorimetric detection.

Nucleic acids may also be isolated, purified, detected and/or subjected to recombinant DNA technology using nucleic acid sequence amplification techniques.

Suitable nucleic acid amplification techniques covering both thermal and isothermal methods are well known to the skilled addressee, and include polymerase chain reaction (PCR); strand displacement amplification (SDA); rolling circle replication (RCR); nucleic acid sequence-based amplification (NASBA), Q-β replicase amplification, recombinase polymerase amplification (RPA) and helicase-dependent amplification, although without limitation thereto.

As used herein, an “amplification product” refers to a nucleic acid product generated by nucleic acid amplification.

Nucleic acid amplification techniques may include particular quantitative and semi-quantitative techniques such as qPCR, real-time PCR and competitive PCR, as are well known in the art.

In still a further aspect, the invention provides a genetic construct comprising the nucleic acid of the previous aspect.

In particular embodiments, the genetic construct comprises the nucleic acid operably linked or connected to one or more other genetic components. A genetic construct may be suitable for therapeutic delivery of the nucleic acid or for recombinant production of the fusion protein of the invention in a host cell.

Broadly, the genetic construct can be in the form of, or comprises genetic components of, a plasmid, bacteriophage, a cosmid, a yeast or bacterial artificial chromosome as are well understood in the art. Genetic constructs may be suitable for maintenance and propagation of the nucleic acid in bacteria or other host cells, for manipulation by recombinant DNA technology and/or expression of the nucleic acid or an encoded protein of the invention.

For the purposes of host cell expression, the genetic construct is an expression construct. Suitably, the expression construct comprises the nucleic acid of the invention operably linked to one or more additional sequences in an expression vector. An “expression vector” may be either a self-replicating extra-chromosomal vector such as a plasmid, or a vector that integrates into a host genome.

By “operably linked” is meant that said additional nucleotide sequence(s) is/are positioned relative to the nucleic acid of the invention preferably to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, polyadenylation sequences, transcriptional start and termination sequences, translational start and termination sequences, and enhancer or activator sequences. Constitutive, repressible or inducible promoters as known in the art are contemplated by the invention.

The expression construct may also include an additional nucleotide sequence encoding a fusion partner (typically provided by the expression vector) so that the recombinant protein is expressed as a fusion protein.

The expression construct may also include an additional nucleotide sequence encoding a selection marker such as amp^{R}, neo^{R} or kan^{R}, although without limitation thereto.

In particular embodiments, the expression construct may be in the form of plasmid DNA, suitably comprising a promoter operable in an animal cell (e.g., a CMV, an A-crystallin or SV40 promoter). In other embodiments, the nucleic acid may be in the form of a viral construct such as an adenoviral, vaccinia, lentiviral or adeno-associated viral vector.

In another aspect, the invention relates to a host cell transformed with a nucleic acid molecule or a genetic construct described herein. Suitable host cells for expression may be prokaryotic or eukaryotic. For example, suitable host cells may include but are not limited to mammalian cells (e.g., HeLa, Cos, NIH-3T3, HEK293T, Jurkat cells), yeast cells (e.g., Saccharomyces cerevisiae), insect cells (e.g., Sf9, Trichoplusia ni) utilized with or without a baculovirus expression system, plant cells (e.g., Chlamydomonas reinhardtii, Phaeodactylum tricornutum) or bacterial cells, such as E. coli. Introduction of genetic constructs into host cells (whether prokaryotic or eukaryotic) is well known in the art, as for example described in CURRENT PROTOCOLS IN MOLECULAR BIOLOGY Eds. Ausubel et al., (John Wiley & Sons, Inc. 1995-2015), in particular Chapters 9 and 16.

Related aspects of the invention provide a method of producing the fusion protein described herein, including the steps of; (i) culturing the host cell of the previous aspect; and (ii) isolating said fusion protein from said host cell cultured in step (i).

In this regard, the recombinant protein may be conveniently prepared by a person skilled in the art using standard protocols, such as those hereinbefore provided.

In yet another aspect, the invention resides in a fusion protein produced according to the aforementioned aspect.

In still yet another aspect, the invention relates to a kit comprising the fusion protein described herein and optionally instructions for use.

In some embodiments, the kit is suitable for use in the method/s of the aforementioned aspects. Accordingly, the kit may comprise additional reagents, such as a buffer, first and/or second proteases, a detergent, solubilised phospholipids and reagents and/or a substrate for isolating the enzyme domain.

Preferred embodiments of the invention are defined in the paragraphs below. Context permitting, the features of any paragraph may be read in combination with any other paragraph or paragraphs. Likewise, features of a product may be features of a method, and vice-versa. Also, context permitting, the features of any paragraph below may be read in combination with any other paragraph or paragraphs located elsewhere in this specification, particularly the DETAILED DESCRIPTION OF THE INVENTION and the CLAIMS.
1. A non-cyclised/non-circularised fusion protein comprising a target protein flanked by at least one circularisation site and an enzyme domain that, by way of a unimolecular reaction, facilitates cyclising/circularisation of the target protein upon recognition and binding of the at least one circularisation site by the enzyme domain.
2. A fusion protein capable of producing a circularised form of a target protein, the fusion protein comprising:
the target protein;
at least one circularisation site adjacent the target protein; and
an enzyme domain capable of interacting with the at least one circularisation site and circularising the target protein.
3. The target protein is a peptide, polypeptide or protein.
4. The fusion protein integrates protein expression, purification and circularisation into one single molecule, which advantageously facilitates the production process at any scale.
5. The fusion protein is linear, not circular.
6. The target protein is circularised by way of a unimolecular reaction. In some embodiments, circularisation can be achieved at theoretically infinitely low concentrations without loss in reaction rate. Thus, the reaction is preferably insensitive to scale and produces improve yields of cyclic target protein products compared to known methods.
7. The target protein of the fusion protein is circularised, initially by way of a unimolecular reaction.
8. The target protein of the fusion protein is circularised by way of an enzyme domain released from a like said fusion protein.
9. The target protein, once circularised, is identical in sequence to the target protein as used in the fusion protein.
10. The target protein, once circularised, is not identical in sequence to the target protein as used in the fusion protein in that it contains one or more additional amino acids of a non-target protein region of said fusion protein.
11. The target protein is or comprises a membrane scaffold protein (MSP), or a fragment, variant or derivative thereof.
12. The circularised MSP or fragment, variant or derivative thereof is identical in sequence to the target protein as used in the fusion protein.
13. The target protein is or comprises a cyclotide, or a fragment, variant or derivative thereof.
14. The cyclotide is SFTI, Vc1.1, Kalata B1 or MCOTI-II, or an orthologue, fragment, variant or derivative thereof.
15. The circularised cyclotide is not identical in sequence to the target protein as used in the fusion protein.
16. The circularised MSP or fragment, variant or derivative thereof is suitable for use in the production of a nanodisc.
17. The diameter of a circularised MSP or fragment, variant or derivative thereof is between about 5 nm to about 80 nm.
18. The cyclic form of the target protein is for binding to a target of interest, such as a therapeutic target or pesticide target.
19. The target of interest is a biomacromolecule such as a protein, a peptide, a nucleic acid, a polycarbohydrate, or a small molecule such as an organic compound or an organometallic complex, or any other molecule that contributes to a disease or is a target of a pesticide.
20. The enzyme domain activity is modulated by the introduction of at least one mutation.
21. The modulated activity results in: i) increased or reduced catalytic activity; ii) increased or reduced binding to the at least one circularisation site; and/or iii) an altered circularisation site, (for some embodiments, preferably other than the motif/site LPXTG).
22. Suitable mutations that reduce the catalytic activity by any one of or any combination of i) to iii) are listed in Table 4.
23. The enzyme domain comprises at least one ligase or cyclase, or an enzymatically active fragment, variant or derivative thereof.
24. The enzyme domain is a sortase or an asparaginyl endopeptidase (AEP), or a combination thereof, or an enzymatically active fragment, variant or derivative thereof.
25. The ligase is or comprises a sortase or an enzymatically active fragment, variant or derivative thereof.
26. The sortase is Staphylococcus aureus wild type sortase A, evolved sortase (eSrtA), eSrtA(2A-9), eSrtA(4S-9), or Streptococcus pyogenes sortase A, or an enzymatically active fragment, variant, derivative or orthologue thereof.
27. The at least one circularisation site is a single circularisation site situated adjacent the N- or C-terminal end of the target protein.
28. The at least one circularisation site comprises first and second circularisation sites adjacent respective N- and C-terminal ends of the target protein.
29. The at least one circularisation site comprises first and second circularisation sites adjacent respective C- and N-terminal ends of the target protein.
30. The at least one circularisation site comprises a sortase acceptor motif and/or a sortase recognition motif.
31. The first circularisation site is or comprises a sortase acceptor motif.
32. The sortase acceptor motif is located at, near, adjacent or towards the N- or C-terminus of the fusion protein.
33. The first circularisation site comprises anon-polar amino acid sequence.
34. The non-polar amino acid sequence consists of 1 to 20 amino acids.
35. The non-polar amino acid sequence comprises one or a plurality of glycines and/or alanines.
36. The non-polar amino acid sequence comprises Gly-[Gly]ₙ, wherein n=0-5, or Ala-[Ala]ₙ, wherein n=0-5.
37. The second circularisation site is or comprises a sortase recognition motif.
38. The sortase recognition motif is located at, near, adjacent or towards the N- or C-terminus of the fusion protein.
39. The second circularisation site comprises an amino acid sequence of LPXTG/A, LAXTG, LPXSG, APXTG, FPXTG or LMVGG, where X represents any amino acid.
40. For SrtA, the sortase recognition motif comprises LPXTG/A, LAXTG or LPXSG, where X is any amino acid.
41. For SrtA the sortase recognition motif comprises the amino acid sequence LPGTG/A, LPSTG/A, LPETG/A, LPGTG, LPGTA, LPSTG, LPSTA, LPETG or LPETA.
42. For eSrtA(4S-9) the sortase recognition motif comprises LPXSG; for eSrtA(2A-9) the sortase recognition motif comprises LAXTG; for SrtA-F40 or SrtA-A1-22 the sortase recognition motif comprises APXTG; for SrtA-F1-20 the sortase recognition motif comprises FPXTG; and, for SrtAβ the sortase recognition motif comprises LMVGG, where X is any amino acid.
43. Activation of the sortase enzyme or enzymatically active fragment, variant or derivative thereof is calcium dependent.
44. Activation of the sortase enzyme comprises the step of adding calcium.
45. The cyclase is or comprises an asparaginyl endopeptidase (AEP) enzyme or an enzymatically active fragment, variant or derivative thereof.
46. The AEP enzyme is butelase-1, or an enzymatically active fragment, variant, derivative or orthologue thereof.
47. The at least one circularisation site comprises a butelase-1 recognition motif.
48. The butelase-1 recognition motif is located at or adjacent the C-terminus of the target protein.
49. The butelase-1 recognition motif comprises an amino acid sequence of Asp-His-Val or Asn-His-Val.
50. The AEP enzyme is OaAEP, or an enzymatically active fragment, variant, derivative or orthologue thereof.
51. The at least one circularisation site comprises an OaAEP recognition motif.
52. The OaAEP recognition motif is located at or adjacent the C-terminus of the target protein.
53. The first circularisation site is or comprises the OaAEP recognition motif located at or adjacent the C-terminus of the target protein.
54. The at least one circularisation site comprises an amino acid sequence of Asn-Gly-Leu.
55. The at least one circularisation site of the fusion protein comprises an AEP recognition motif, comprising the amino acid sequence X₁X₂X₃, where X₁ is N or D; X₂ is G or S; and X₃ is L, A or I.
56. The second circularisation site is or comprises an AEP acceptor motif.
57. The AEP acceptor motif is located at or adjacent a C-terminus of the target protein.
58. The AEP acceptor motif comprises the amino acid sequence X₄X₅, where X₄ is optional and any amino acid or G, Q, K, V or L; and X₅ is optional or any amino acid or L, F or I or a hydrophobic amino acid residue.
59. The AEP acceptor motif is or comprises the amino acid sequence GL.
60. The fusion protein comprises at least one spacer.
61. The at least one spacer is situated between the target protein and the enzyme domain.
62. The spacer comprises: a glycine polymer (G)ₙ where n is an integer of at least one, two, three, four or five; glycine-serine polymer (G₁₋₅S₁₋₅)ₙ, where n is an integer of at least one, two, three, four or five; a glycine-alanine polymer; or, an alanine-serine polymer.
63. The spacer is between 1 and 30 amino acid residues in length.
64. The spacer is between 5-20 amino acid residues in length.
65. The spacer has the amino acid sequence GGS, GS(GGS)ₙ, GAAA or LEGT, where n is at least one.
66. The spacer is approximately 35 to 45 Angstroms (Å) in length.
67. The fusion protein may further comprise at least one inhibitory domain.
68. The at least one inhibitory domain is adjacent one or more of the at least one circularisation site.
69. The at least one inhibitory domain is positioned at, near, adjacent or towards an N- or C-terminus of the fusion protein.
70. The at least one inhibitory domain comprises a cap sequence adjacent the at least one circularisation site.
71. The at least one inhibitory domain is configured to inhibit recognition of the at least one circularisation site by the enzyme domain.
72. The at least one inhibitory domain comprises an enzyme inhibitory sequence configured to inhibit activity of the enzyme domain by contacting the enzyme domain directly.
73. The fusion protein comprises an inhibitory domain positioned N-terminally to a first said circularisation site positioned at, near, adjacent or towards an N-terminus of the fusion protein, such that cleavage of the inhibitory domain exposes the circularisation site as the N-terminus of the fusion protein and/or removes the enzyme inhibitory sequence from the fusion protein.
74. The fusion protein comprises an inhibitory domain positioned C-terminally to a circularisation domain positioned at, near, adjacent or towards a C-terminus of the fusion protein, such that cleavage of the inhibitory domain exposes the circularisation site as the C-terminus of the fusion protein and/or removes the enzyme inhibitory sequence from the fusion protein.
75. The inhibitory domain is capable of being removed or cleaved to release the enzyme domain inhibitory sequence and/or expose the at least one circularisation site adjacent thereto as the N- or C-terminus of the fusion protein.
76. The inhibitory domain is positioned adjacent a first circularisation site, wherein the first circularisation site comprises one or a plurality of glycine residues and is positioned N-terminally to the target protein.
77. The inhibitory domain comprises a protease recognition sequence that is recognized and cleaved by a protease.
78. The inhibitory domain comprises the recognition sequence Leu-Val-Pro-Arg-Gly-Ser, which is recognized and cleaved by thrombin.
79. The inhibitory domain comprises the recognition sequence Glu-Asn-Leu-Tyr-Phe-Gln-(Gly/Ser), which is recognized and cleaved by TEV protease.
80. The inhibitory domain comprises a first protease cleavage site cleavable by a first protease to expose the at least one circularisation site adjacent thereto and release inhibition of recognition and binding of the at least one circularisation site by the enzyme domain.
81. The inhibitory domain comprises an amino acid sequence containing Met-Gly which is cleaved by bacterial Met aminopeptidase (MAP) to expose the at least one circularisation site adjacent thereto and release inhibition of recognition and binding of the at least one circularisation site by the enzyme domain.
82. The fusion protein comprises at least one affinity/purification tag at or towards an N- and/or C-terminus thereof and adjacent the enzyme domain.
83. There is a first affinity tag at, near, adjacent or towards the N-terminus of the fusion protein and a second affinity tag at, near, adjacent or towards the C-terminus of the fusion protein.
84. The fusion protein further comprises a second protease cleavage site positioned between the at least one spacer and the enzyme domain, the second protease cleavage site cleavable by a second protease to facilitate removal of the enzyme domain from the fusion protein or spacer following circularisation of the target protein.
85. The second protease cleavage site forms at least part of the at least one spacer.
86. A fusion protein comprising from N-terminus to C-terminus:
optionally an inhibitory domain comprising a first protease cleavage site;
optionally a first circularisation site, such as a sortase acceptor motif or an OaAEP acceptor motif;
a target protein;
a second circularisation site, such as a sortase recognition motif, an OaAEP recognition motif or a butelase recognition motif;
optionally a spacer;
optionally a second protease cleavage site;
an enzyme domain; and
optionally at least one affinity tag.
87. The fusion protein as defined above comprises an inhibitory domain comprising a first protease cleavage site.
88. The fusion protein as defined above comprises a first circularisation site, such as a sortase acceptor motif.
89. The fusion protein as defined above comprises an OaAEP acceptor motif.
90. The fusion protein as defined above comprises a spacer.
91. The fusion protein as defined above comprises a second protease cleavage site.
92. The fusion protein as defined above comprises an affinity tag.
93. The fusion protein as defined above comprises an amino acid sequence set forth in any one of SEQ ID NOs:1 to 21 or in any one of FIGS. 1, 4, 7, 14, 15 and 16, or a fragment, variant, derivative or orthologue thereof.
94. The fusion protein as defined above is encoded by the nucleotide sequence set forth in any one of SEQ ID NOs: 22 to 34 or in any one of FIGS. 7, 14, 15 and 16, or a fragment, variant, derivative or orthologue thereof.
95. A nanodisc comprising the cyclised/circularised MSP target protein or fragment, variant, derivative or orthologue thereof as defined above.
96. An isolated nucleic acid encoding the fusion protein as defined above.
97. A genetic construct comprising the nucleic acid as defined above.
98. A host cell comprising the nucleic acid and/or the genetic construct as defined above.
99. A method for circularising a target protein, said method including the steps of:
(a) providing a fusion protein comprising:
a target protein;
at least one circularisation site adjacent the target protein;
an enzyme domain capable of circularising the target protein; and
optionally a spacer positioned between the target protein and the enzyme domain;
(b) facilitating interaction of the enzyme domain with the at least one circularisation site to thereby circularise the amino acid sequence of the target protein.
100. The fusion protein may have one or more features as defined above.
101. A method of producing a nanodisc, said method including the steps of:
(a) providing a fusion protein comprising:
a membrane scaffold protein or fragment thereof;
at least one circularisation site adjacent the membrane scaffold protein or fragment thereof;
an enzyme domain capable of circularising the membrane scaffold protein or fragment thereof; and
optionally a spacer positioned between the membrane scaffold protein or fragment thereof and the enzyme domain;
(b) optionally facilitating interaction of the enzyme domain with the at least one circularisation site to thereby circularise the membrane scaffold protein or fragment thereof; and
(c) contacting the fusion protein of step (a) or the circularised membrane scaffold protein of step (b) with a lipophilic molecule(s) to thereby produce the nanodisc.
102. Step (b) above is performed in the presence of calcium ions and/or a detergent, for catalysis by the enzyme domain.
103. The method's fusion protein and the membrane scaffold protein or fragment, variant or derivative thereof may have one or more features as defined above.
104. The method further includes an initial step of producing the fusion protein or the membrane scaffold protein or fragment, variant or derivative thereof.
105. The step of facilitating interaction of the enzyme domain with the at least one circularisation site comprises the step of activating the enzyme domain.
106. The step of facilitating interaction of the enzyme domain with the at least one circularisation site comprises the step of removing an inhibitory domain adjacent one or more of the at least one circularisation site.
107. The fusion protein includes a spacer and the method further includes a subsequent step of removing the spacer from the enzyme domain.
108. The method further includes the subsequent step of isolating or purifying the enzyme domain removed from a remainder of the fusion protein or the membrane scaffold protein or fragment, variant or derivative thereof by way of an affinity tag positioned at or towards an N- or C-terminus of the fusion protein or the membrane scaffold protein or fragment, variant or derivative thereof and adjacent the enzyme domain.
109. In the method, the target protein is circularised by way of a unimolecular reaction. In some embodiments, circularisation can be achieved at theoretically infinitely low concentrations without loss in reaction rate. In some embodiments, the reaction is insensitive to scale and produces improve yields of cyclic target protein products compared to known techniques.
110. In the method, the target protein is circularised, initially by way of a unimolecular reaction.
111. In the method, the target protein of the fusion protein is circularised by way of an enzyme domain released from a like said fusion protein.
112. The method includes the step of modulating activity of the enzyme domain.
113. Activity of the enzyme domain is modulated by the introduction of at least one mutation as a result of structure-activity-relationship (SAR).
114. The altered enzyme activity can result in: i) increased or reduced catalytic activity; ii) increased or reduced binding to the at least one circularisation site (ie. enzyme recognition site); or iii) an altered circularisation site (ie. recognition site), for some embodiments preferably other than the motif/site LPXTG.
115. Mutations that reduce the enzyme activity by any one of or any combination of i) to iii) are listed in Table 4.

116. A circularised target protein or nanodisc produced according to each method described above.

Yet further preferred embodiments of the invention are defined in the paragraphs below. Context permitting, the features of any paragraph below may be read in combination with any other paragraph or paragraphs below and also elsewhere in this specification, particularly the DETAILED DESCRIPTION OF THE INVENTION and the CLAIMS. Likewise, features of a product may be features of a method, and vice-versa.
1. A fusion protein capable of producing a circularised form of a target protein, the fusion protein comprising:
the target protein;
at least one circularisation site adjacent the target protein; and
an enzyme domain capable of interacting with the at least one circularisation site and circularising the target protein.
2. The fusion protein of paragraph 1, further comprising a spacer positioned between the amino acid sequence of the target protein and the enzyme domain.
3. The fusion protein of paragraph 1 or paragraph 2, wherein the enzyme domain comprises an amino acid sequence of a ligase or cyclase or an enzymatically active fragment, variant or derivative thereof.
4. The fusion protein of paragraph 3, wherein the ligase or cyclase is selected from the group consisting of a sortase, an asparaginyl endopeptidase (AEP), such as butelase 1 and O. affinis AEP (OaAEP), and any combination thereof.
5. The fusion protein of any one of the preceding paragraphs, wherein enzyme domain activity is modulated by the introduction of at least one mutation.
6. The fusion protein of any one of the preceding paragraphs, wherein the at least one circularisation site comprises a first and a second circularisation site/domain adjacent respectively to N- and C-terminal ends of the amino acid sequence of the target protein.
7. The fusion protein of paragraph 6, wherein:
the first circularisation site comprises a non-polar amino acid sequence comprising one or a plurality of glycines or alanines; and
the second circularisation site comprises an amino acid sequence of LPXTG/A, LAXTG, LPXSG, APxTG, FPxTG or LMVGG, where X represents any amino acid.
8. The fusion protein of any one of the preceding paragraphs, further comprising an inhibitory domain adjacent one or more of the at least one circularisation site and optionally wherein the inhibitory domain is positioned at or towards an N- or C-terminus of the fusion protein.
9. The fusion protein of paragraph 8, wherein the inhibitory domain comprises a first protease cleavage site cleavable by a first protease to expose the at least one circularisation sites adjacent thereto.
10. The fusion protein of any one of the preceding paragraphs, further comprising one or a plurality of affinity tags at or towards an N- and/or C-terminus thereof and adjacent the enzyme domain.
11. The fusion protein of any one of the preceding paragraphs, further comprising a second protease cleavage site positioned between the spacer and the enzyme domain, the second protease cleavage site cleavable by a second protease to facilitate removal of the enzyme domain from the fusion protein.
12. The fusion protein of any one of the preceding paragraphs, wherein the target protein is or comprises a membrane scaffold protein, a therapeutic protein or a target of a pesticide, such as SFTI, Vc1.1, Kalata B1 and MCOTI-II, or a fragment, variant or derivative thereof.
13. The fusion protein of any one of the preceding paragraphs, comprising the amino acid sequence set forth in any of SEQ ID NOs: 1 to 21 or a fragment, variant or derivative thereof.
14. An isolated nucleic acid encoding the fusion protein of any one of paragraphs 1 to 13.
15. A genetic construct comprising the isolated nucleic acid of paragraph 14.
16. A host cell comprising the isolated nucleic acid of paragraph 14 and/or the genetic construct of paragraph 15.
17. A method of producing the fusion protein of any one of paragraphs 1 to 13, including the steps of
(i) culturing the host cell of paragraph 16; and
(ii) isolating said fusion protein from said host cell cultured in step (i).
18. A method for circularising a target protein, said method including the steps of
(a) providing a fusion protein comprising:
a target protein;
at least one circularisation site adjacent the target protein;
an enzyme domain capable of circularising the target protein; and
optionally a spacer positioned between the target protein and the enzyme domain; and
(b) facilitating interaction of the enzyme domain with the at least one circularisation site to thereby circularise the amino acid sequence of the target protein.
19. A method of producing a nanodisc, said method including the steps of
(a) providing a fusion protein comprising:
a target protein comprising a membrane scaffold protein or fragment thereof;
at least one circularisation site adjacent the membrane scaffold protein or fragment thereof;
an enzyme domain capable of circularising the membrane scaffold protein or fragment thereof; and
optionally a spacer positioned between the membrane scaffold protein or fragment thereof and the enzyme domain;
(b) optionally facilitating interaction of the enzyme domain with the at least one circularisation site to thereby circularise the membrane scaffold protein or fragment thereof; and
(c) contacting the fusion protein of step (a) or the circularised membrane scaffold protein or fragment thereof of step (b) with a lipophilic molecule(s) to thereby produce the nanodisc.
20. The method of paragraph 18 or paragraph 19, wherein the fusion protein is that of any one of paragraphs 1 to 13 or the membrane scaffold protein or fragment thereof is that of paragraph 12 or paragraph 13.
21. The method of any one of paragraphs 18 to 20, further including the initial step of producing the fusion protein or the membrane scaffold protein or fragment thereof.
22. The method of any one of paragraphs 18 to 21, wherein the step of facilitating interaction of the enzyme domain with the at least one circularisation site comprises the step of activating the enzyme domain.
23. The method of any one of paragraphs 18 to 22, wherein the step of facilitating interaction of the enzyme domain with the at least one circularisation site comprises the step of removing an inhibitory domain adjacent one or more of the at least one circularisation site.
24. The method of any one of paragraphs 18 to 23, further including the subsequent step of removing the spacer from the enzyme domain.
25. The method of any one of paragraphs 18 to 24, further including the subsequent step of isolating or purifying the enzyme domain removed from the fusion protein or the membrane scaffold protein or fragment thereof by way of an affinity tag positioned at or towards an N- or C-terminus of the fusion protein or the membrane scaffold protein or fragment thereof and adjacent the enzyme domain.
26. A cyclic/circularised protein or nanodisc produced according to the method of any one of paragraphs 18 to 25.

Yet further preferred embodiments of the invention are defined in the paragraphs below. Context permitting, the features of any paragraph below may be read in combination with any other paragraph or paragraphs below and also elsewhere in this specification, particularly the DETAILED DESCRIPTION OF THE INVENTION and the CLAIMS. Likewise, features of a product may be features of a method, and vice-versa.
1. A non-circularised fusion protein comprising a target protein flanked by at least one circularisation site and an enzyme domain that, by way of a unimolecular reaction, facilitates circularisation of the target protein upon recognition and binding of the at least one circularisation site by the enzyme domain.
2. A fusion protein capable of producing a circularised form of a target protein, the fusion protein comprising:
the target protein;
at least one circularisation site adjacent the target protein; and
an enzyme domain capable of interacting with the at least one circularisation site and circularising the target protein.
3. The fusion protein of paragraph 2, wherein the target protein is circularised by way of a unimolecular reaction.
4. The fusion protein of paragraph 1, 2 or 3, wherein the fusion protein integrates protein expression, purification and circularisation into one single molecule.
5. The fusion protein of paragraph 1, 2, 3 or 4, wherein said target protein is a peptide, polypeptide or protein.
6. The fusion protein of any one of paragraphs 1-5, wherein the enzyme domain comprises at least one ligase or cyclase, or an enzymatically active fragment, variant or derivative thereof.
7. The fusion protein of paragraph 6, wherein the at least one circularisation site comprises first and second circularisation sites adjacent respective terminal ends of the target protein.
8. The fusion protein of paragraph 6 or 7, wherein the enzyme domain is a sortase or an asparaginyl endopeptidase (AEP), or a combination thereof, or an enzymatically active fragment, variant or derivative thereof.
9. The fusion protein of any one of paragraphs 1-8, further comprising at least one spacer, wherein preferably the at least one spacer is situated between the target protein and the enzyme domain.
10. The fusion protein of any one of paragraphs 1-9, further comprising at least one inhibitory domain, wherein preferably the at least one inhibitory domain is adjacent one or more of the at least one circularisation site, and optionally the at least one inhibitory domain is positioned at, near, adjacent or towards an N- or C-terminus of the fusion protein.
11. The fusion protein of any one of paragraphs 1-10, wherein the circularised form of the target protein is capable of binding to a target of interest, such as a therapeutic target or pesticide target, wherein preferably the target of interest is a biomacromolecule, such as a protein, a peptide, a nucleic acid, a polycarbohydrate, or a small molecule such as an organic compound or an organometallic complex, or any other molecule that contributes to a disease or is a target of a pesticide.
12. The fusion protein of any one of paragraphs 1-11, wherein the target protein is or comprises a membrane scaffold protein (MSP), or a fragment, variant or derivative thereof, wherein preferably a circularised MSP or fragment, variant or derivative thereof is capable of being used in the production of a nanodisc.
13. The fusion protein of any one of paragraphs 1-11, wherein the target protein is or comprises a cyclotide, or a fragment, variant or derivative thereof, wherein preferably the cyclotide is SFTI, Vc1.1, Kalata B1 or MCOTI-II, or an orthologue, fragment, variant or derivative thereof.
14. A fusion protein:
(i) comprising or consisting of an amino acid sequence set forth in any one of SEQ ID NOs:1 to 21 or in any one of FIGS. 1, 4, 7, 14, 15 and 16, or a self-circularising fragment, variant, derivative or orthologue thereof; or
(ii) being encoded by a nucleotide sequence set forth in any one of SEQ ID NOs: 22 to 34 or in any one of FIGS. 7, 14, 15 and 16, or a fragment, variant, derivative or orthologue thereof.
15. A fusion protein comprising from N-terminus to C-terminus:
optionally an inhibitory domain comprising a first protease cleavage site;
optionally a first circularisation site, such as a sortase acceptor motif or an OaAEP acceptor motif;
a target protein;
a second circularisation site, such as a sortase recognition motif, an OaAEP recognition motif or a butelase recognition motif;
optionally a spacer;
optionally a second protease cleavage site;
an enzyme domain; and
optionally at least one affinity tag.
16. The fusion protein of paragraph 15, wherein:
(i) the fusion protein comprises an amino acid sequence set forth in any one of SEQ ID NOs:1-21 or in any one of FIGS. 1, 4, 7, 14, 15 and 16, or a fragment, variant, derivative or orthologue thereof; or
(ii) the fusion protein is encoded by the nucleotide sequence set forth in any one of SEQ ID NOs: 22-34 or in any one of FIGS. 7, 14, 15 and 16, or a fragment, variant, derivative or orthologue thereof.
17. An isolated nucleic acid [1] encoding the fusion protein of any one of paragraphs 1 to 16; a genetic construct [2] comprising said nucleic acid [1]; or, a host cell comprising said nucleic acid [1] and/or said genetic construct [2].

18. A method of producing the fusion protein of any one of paragraphs 1-16, including the steps of:
(i) culturing the host cell of paragraph 17; and
(ii) isolating said fusion protein from said host cell cultured in step (i).
19. A method for circularising a target protein, said method including the steps of:
(a) providing a fusion protein comprising:
   a target protein;
   at least one circularisation site adjacent the target protein;
   an enzyme domain capable of circularising the target protein; and
   optionally a spacer positioned between the target protein and the enzyme domain; and
(b) facilitating interaction of the enzyme domain with the at least one circularisation site to thereby circularise the amino acid sequence of the target protein.
20. A method of producing a nanodisc, said method including the steps of:
(a) providing a fusion protein comprising:
   a target protein comprising a membrane scaffold protein or fragment thereof;
   at least one circularisation site adjacent the membrane scaffold protein or fragment thereof;
   an enzyme domain capable of circularising the membrane scaffold protein or fragment thereof; and
   optionally a spacer positioned between the membrane scaffold protein or fragment thereof and the enzyme domain;
(b) optionally facilitating interaction of the enzyme domain with the at least one circularisation site to thereby circularise the membrane scaffold protein or fragment thereof, and
(c) contacting the fusion protein of step (a) or the circularised membrane scaffold protein or fragment thereof of step (b) with a lipophilic molecule(s) to thereby produce the nanodisc.
21. The method of paragraph 20, wherein said fusion protein has one or more features as described in any one of paragraphs 1-12 and 14-16 in so far as they relate to a membrane scaffold protein or fragment thereof.
22. The method of paragraph 19, 20 or 21, comprising the step of modulating activity of the enzyme domain by introducing at least one mutation into the enzyme domain, wherein modulated activity results in: i) increased or reduced catalytic activity; ii) increased or reduced binding to the at least one circularisation site; and/or iii) an altered circularisation site.
23. The method of paragraph 19, 20, 21 or 22, wherein the target protein of the fusion protein is circularised by way of an enzyme domain released from a like said fusion protein.
24. The method of paragraph 19, 20, 21, 22 or 23:
(i) further including an initial step of producing the fusion protein;
(ii) wherein the step of facilitating interaction of the enzyme domain with the at least one circularisation site comprises the step of activating the enzyme domain;
(iii) wherein the step of facilitating interaction of the enzyme domain with the at least one circularisation site comprises the step of removing an inhibitory domain adjacent one or more of the at least one circularisation site;
(iv) further including a subsequent step of removing the spacer from the enzyme domain;
(v) further including a subsequent step of isolating or purifying the enzyme domain removed from the fusion protein by way of an affinity tag positioned at or towards an N- or C-terminus of the fusion protein and adjacent the enzyme domain.
25. A circularised target protein produced by the method of any one of paragraphs 19 and 21-24 when dependent on paragraph 19, or a nanodisc produced by the method of any one of paragraphs 20-24 when dependent on paragraph 20.

All computer programs, algorithms, patent and scientific literature referred to herein is incorporated herein by reference.

The reference to any prior art in this specification is not, and should not be taken as an acknowledgement or any form of suggestion that the prior art forms part of the common general knowledge.

As used herein, unless the context requires otherwise, the words “comprise”, “comprises” and “comprising” will be understood to mean the inclusion of a stated integer or group of integers but not the exclusion of any other integer or group of integers.

The indefinite articles ‘a’ and ‘an’ are used here to refer to or encompass singular or plural elements or features and should not be taken as meaning or defining “one” or a “single” element or feature.

As generally used herein “about” refers to a tolerance or variation in a stated value or amount that does not appreciably or substantially affect function, activity or efficacy. Typically, the tolerance or variation is no more than 10%, 5%, 3%, 2%, or 1% above or below a stated value or amount.

So that the invention may be readily understood and put into practical effect, embodiments of the invention will be described with reference to the following non-limiting Examples.

### BRIEF DESCRIPTION OF THE FIGURES

FIG. 1: Autocyclase as a bio-platform to integrate protein expression, purification and cyclisation. (A). Target proteins of different sizes are expressed as fusion proteins comprising sortase, and will undergo an auto-cyclisation process to generate cyclised target proteins. (B). Construct design of the autocyclase for cyclising linear target proteins into circular proteins, featuring an N-terminal TEV recognition site (cleavage site 1—ENLYFQG) which generates an N-terminal glycine nucleophile (second circularisation site), target proteins of interest for cyclisation, sortase A recognition site (first circularisation site—LPGTG), an optimized amino acid spacer/linker (GAAALEGT, or GS(GGS)x4), thrombin recognition site (cleavage site 2—LVPRS), Sortase A (enzyme domain) and polyhistidine (affinity) tag (10xH). The fusion protein is subjected to TEV-cleavage (Step 1), resulting in the exposure of a free N-terminal glycine nucleophile. Addition of CaCl₂) selectively activates the autocyclase which mediates the cyclisation and produces a pure cyclic target protein product (Step 2). Thrombin cleavage removes the amino acid linker and recovers sortase A as a pure enzyme without an exposed N-terminal glycine nucleophile (Step 3).

FIG. 2. The linker/spacer design of autocyclase (A). The surface and cartoon display of sortase A solution structure in complex with sortase A recognition site benzylocarbonyl-LPAT* (pdb 2kid) where T* is a threonine analog that replaces the carbonyl group with —CH₂—SH. (B). Five different amino acid linkers/spacers for connecting N-terminus of SrtA and a target protein to be cyclised. If including the flexible segment of srtA, i.e. QAKP, eight amino acids linker, i.e. GAAALEGT, is proposed to be a suitable short linker. Otherwise, a thrombin site, LVPRS, was substituted for QAKP, to combine with GAAALEGT to satisfy the 35 Å distance. For a more relaxed linker with high flexibility, a (GGS)x5 linker was chosen to join the T from LPATG and the first or second amino acid of the enzyme domain. A thrombin site was inserted for the removal of the linker to recycle SrtA enzyme domain. (C-D). The intensity ratio of circular membrane scaffold protein (cMSP) to the fusion protein measured on SDS-page gels. (C). The circularisation of MSP9 with MSP9-L1b/L1-SrtA is more efficient at 37° C. than 23° C. (room temperature). The yield of MSP9-L1b-SrtA with a longer linker is higher than that of MSP-L1-SrtA. (D). The circularisation of MSP9 with MSP9-L2-SrtA is complete in less than 1 hour.

FIG. 3. The production of five different MSP autocyclases. To produce each cMSP, the crude mixture from Ni-NTA purification and after TEV cleavage, is adjusted to a concentration between 50 μM and 100 μM. The reaction is then performed at 37° C. overnight in the presence of 1 mM DDM. Then the reaction mixture is directly loaded onto a Ni-NTA resin, and the flow through is collected, containing the circular MSPs. The mixture before the reaction, after the reaction and the purified cMSPs in the Ni-NTA column flow were analysed in lanes 1, 2 and 3 of the SDS-page gel, respectively. To demonstrate the recycling of SrtA, the L1b linker at the N-terminal end of the L1b-SrtA fusion was removed by thrombin cleavage and analysed in Lane 4 (MSP11 shown). The circularisation of cMSPs were validated by molecular weight (as determined by ESI-MS).

FIG. 4. The production of three cyclic peptides through the autocyclase approach. (A). The autocyclase designs for producing cyclised SFTI, kalataB 1 (kB1) and cVc1.1 are illustrated. The autocyclases enclosed by rectangles in (A) were used to produce cyclic SFTI, kB1 and cVc1.1, as shown in (B). The extra amino acids introduced by the autocyclase approach are highlighted in yellow and underlined. (C). Circularisation and oxidization of the cyclic peptides was confirmed by MALDI.

FIG. 5: The reaction mechanism of autocyclases. (A). Three possible reaction pathways: I. Intramolecular/unimolecular, II. Intermolecular between one fusion and one free sortase A, III. Intermolecular reaction between two fusion proteins. In pathway I and II, the favoured product is a mono-circularised protein. In pathway IIIa tandem “linear protein-LPGTG-linear protein-sortase A” is produced while in pathway IIIb a mono circularised protein is formed as well as one original fusion protein. In pathway I, the enzymatic rate will depend on the concentration of the fusion protein in a unimolecular reaction, following first order kinetics. In pathways II and III, the reaction will proceed via a second order reaction mechanism. (B). The autocyclase L2a-kB1 (including co-purified free sortase A from crude Ni-NTA purification) is assayed for measurement of the reaction rate. The reaction rate doubles when the total concentration is increased from 50 μM to 100 μM, indicating first order kinetics and a unimolecular reaction (pathway I). When the concentration increases to 150 μM, the rate is increased by 4.25 times compared to when the reaction is conducted at 50 μM, indicating that at higher concentrations the reaction is still predominantly first order (pathway I) with evidence of a competing higher order reaction (either pathway I or pathway II).

FIG. 6. (A). Autocyclase-L1-MSP9 and Autocyclase-L1b-MSP9 reaction in the presence of 1 mM DDM at 25° C. and 16° C. for 16 hours. 2 μL from 100 μM reaction or 4 μL from 50 μM is analyzed along with 2 μL 100 μM mixture before the reaction on a 12% SDS-page gel to compare the relative yield of cMSP. (B), (C), and (D). 37° C. reaction for 1 to 6 hours. 1 μL from 100 μM reaction or 2 μL from 50 μM is analyzed along with 1 μL from 100 μM reaction or 2 μL 50 μM mixture before the reaction, to compare the relative product yield of cMSPs. For both autocyclase-L1-MSP9 and autocyclase-L1b-MSP9, the yield of cMSP at 37° C.>23° C.>16° C. At 37° C., the yield of cMSP9 from the autocyclase-L1b-MSP9 is higher than that from autocyclase-L1-MSP9.

FIG. 7. The nucleotide and coding amino acid sequences of His₆-TEV-MSP9-eSrtA after cloning into the pET29 vector. Through synonymous substitution, a XhoI was introduced before the His₆-tag and aKpnI site was introduced between the cMSP9 sequence and the sortase A (pentamutant) sequence to facilitate the replacement of eSrtA by WT-SrtA. Some of the NdeI, KpnI and XhoI sites are highlighted in yellow and underlined.

FIG. 8. (A). The soluble and insoluble fractions of test expression of the G2-His₆-TEV-MSP9-eSrtA fusion at either 30 or 37° C. (B). The comparison of the elution profile after Ni-NTA resin elution from G2-His₆-TEV-MSP9-eSrtA (left) and A2-His₆-TEV-MSP9-eSrtA (right). (C) and (D). The fusion protein expression and stability post IPTG induction of MSP9-SrtA (C) and MSP9-eSrtA (D). The fusion protein was induced by 0.2 mM IPTG at 30° C. Aliquots were taken at 1 h interval over a 6 h time course. The whole cell lysates were harvested from 0.1 mL of culture and lysed in 30 μL of 1×SDS protein loading buffer out of which 8 μL was loaded on a 12% Tris-glycine SDS-page gel for analysis.

FIG. 9. Production, purification and reaction of autocyclases with a poly GGS linker. (A). G2A cNW9 SrtA (Penta) GGS His₁₀. (B). G2A cNW9 SrtA (WT) GGS His₁₀.

FIG. 10. Effect of IPTG concentration on autocyclase expression. Cells were grown in LB media until an optical density of about 1.0 was reached at 37° C. Cultures were induced with either 0.2 mM or 1 mM IPTG at 30° C. The cells were harvested from 0.1 mL of medium. Cell pellets were resuspended in 25 uL 1×SDS loading buffer, boiled at 90° C. for 10 minutes. 4 μL was loaded into each lane. 0.2 mM IPTG is sufficient to produce high yields. (A).

FIG. 11. (A). Expression profile of autocyclases containing different linkers, N-terminal segments and protease sites. (I) The introduction of a 5×GGS linker (363b) increased the hydrolysis and decreases the yield of the fusion protein. (II) The introduction of the inhibitory peptide (373) does not affect yield of the fusion protein (363b) (III). The introduction of a thrombin site (390a) has a small effect on hydrolysis or fusion yield. (B). The introduction of a thrombin site (LVPRS) slightly decreases the yield of the fusion (II). Similarly, autocyclase-L2a-SFTI with the longer linker also experiences slightly more in vivo hydrolysis than autocyclase-L1b-SFTI, seen as weaker fusion and hydrolysis bands below the fusion. There is no detectable in vivo hydrolysis in autocyclase-L1b-SFTI (III).

FIG. 12. Nine optimized sequences for the mRNA translation initiation region.

FIG. 13. (A). Quantitation of reactions rates (using autocyclase-L1b-MSP11 at 37° C.). (B). The relative ratio of cMSP11 against auto-cyclase-L1b-MSP11. (C). Large scale preparation of cMSP11 from auto-cyclase-L1b-MSP11. The flow through from Ni-NTA contains relatively pure cMSP11. On-column thrombin cleavage removes L1b linker and recycles SrtA.

FIG. 14: The nucleotide and coding amino acid sequences of MSP9-LPGT(GGS)x5-SrtA-His₁₀ (A), MSP11-LPGT(GGS)5-wtSrtA-His₁₀ (B), MSP20-LPGT(GGS)5-wtSrtA-His₁₀ (C), MSP7-LPGT(GGS)5-wtSrtA-His₁₀ (D) and MSP6-LPGT(GGS)5-wtSrtA-His₁₀ (E). KpnI and XhoI sites are highlighted in yellow and all capitals, while the TEV, SrtA recognition sites and (GGS)x5 linkers are highlighted in grey and are italicised. H4 helix is highlighted in single underline while H6 helix is highlighted in double underline.

FIG. 15: The nucleotide and coding amino acid sequences of MSP9-LPGTGAAALEGTLVPRS-SrtA-His₁₀ (A), MSP7—LPGTGAAALEGTLVPRS-SrtA-His₁₀ (B), MSP6—LPGTGAAALEGTLVPRS-SrtA-His₁₀ (C), MSP11—LPGTGAAALEGTLVPRS-SrtA-His₁₀ (D) and MSP20—LPGTGAAALEGTLVPRS-SrtA-His10 (E). KpnI and XhoI sites are highlighted in yellow and in capitals while the TEV, SrtA recognition sites and (GGS)x5 linkers are highlighted in grey and are italicised. H4 helix is highlighted in single underline while H6 helix is highlighted in double underline.

FIG. 16: The nucleotide and/or coding amino acid sequences of G-SFTI-LPGT(GGS)5LVPRS-SrtA-His₁₀ (A), G-kB1-LPGT(GGS)5LVPRS-SrtA-His₁₀ (B), G-SFTI-LPGTGAAALEGTLVPRS-SrtA-His₁₀ (C), GGG-SFTI-LPGTGAAALEGTLVPRS-SrtA-His₁₀ (D) and GGG-SFTI-LPGT(GGS)5LVPRS-SrtA-His₁₀ (E), GGG-kB1-LPGT(GGS)5LVPRS-SrtA-His₁₀ (F), GGG-kB1-LPVTGAAALEGTLVPRS-SrtA-His₁₀ (G), G-kB1-LPVTGAAALEGTLVPRS-SrtA-His₁₀ (H), GG-Vc1.1-LPGTGAAALEGTLVPRS-SrtA-His₁₀ (I) and GG-Vc1.1-LPGT(GGS)5LVPRS-SrtA-His₁₀ (J). Cyclotide sequences are highlighted in yellow and double underline while the TEV, SrtA recognition sites and LPGT(GGS)x5LVRPS or LPGTGAAALEGTLVPRS linker are highlighted in grey and single underline.

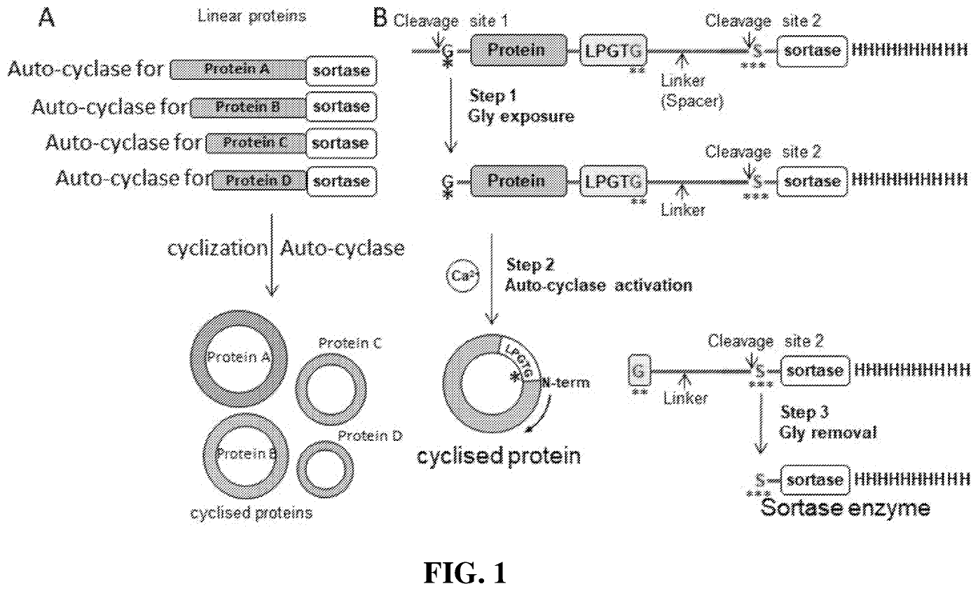

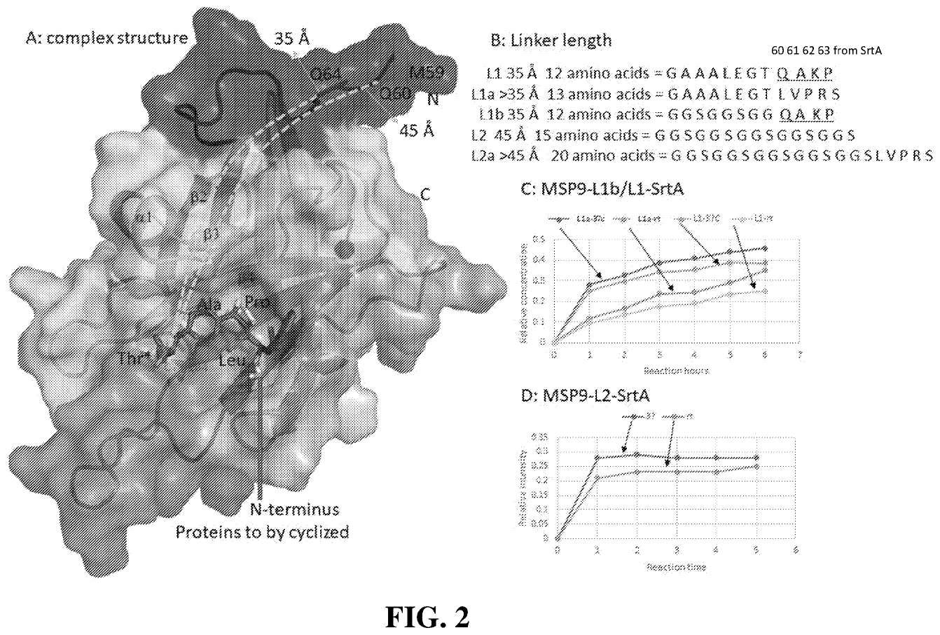

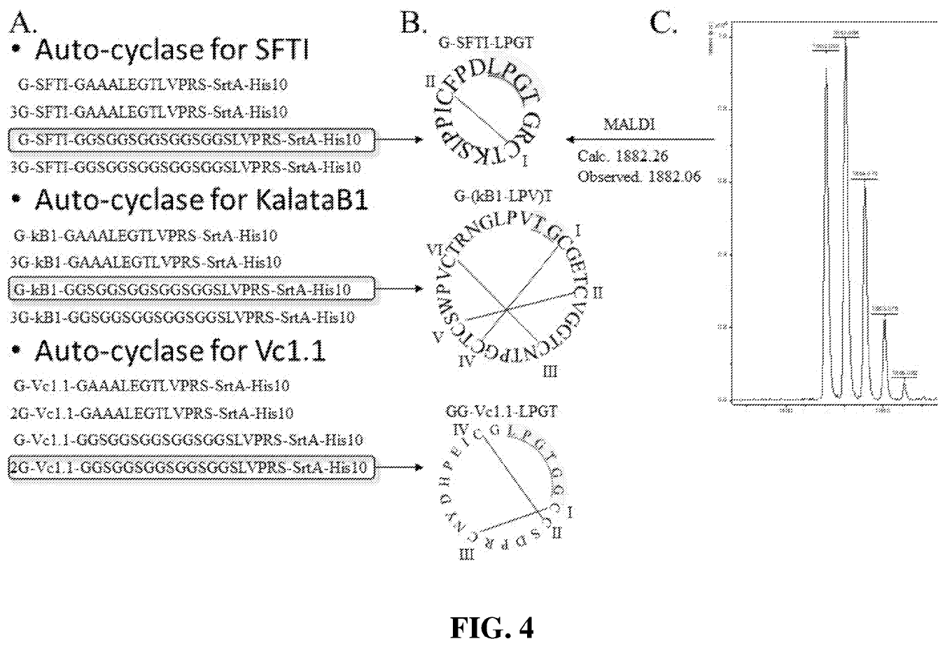

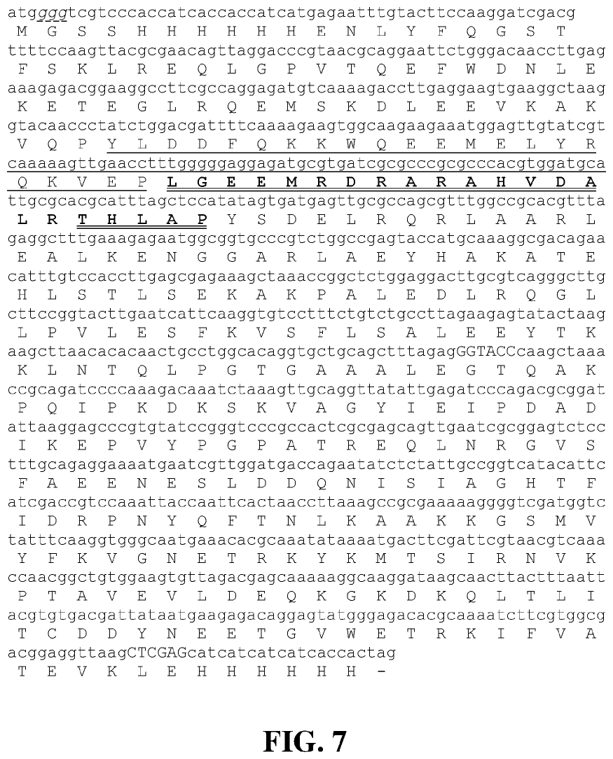

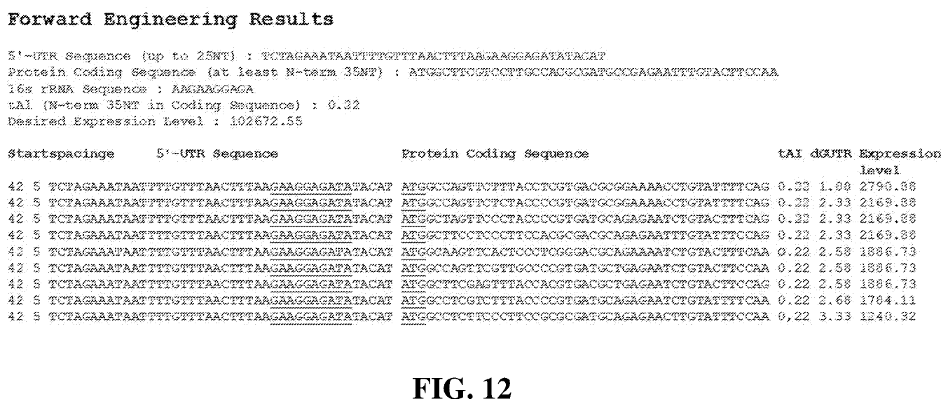

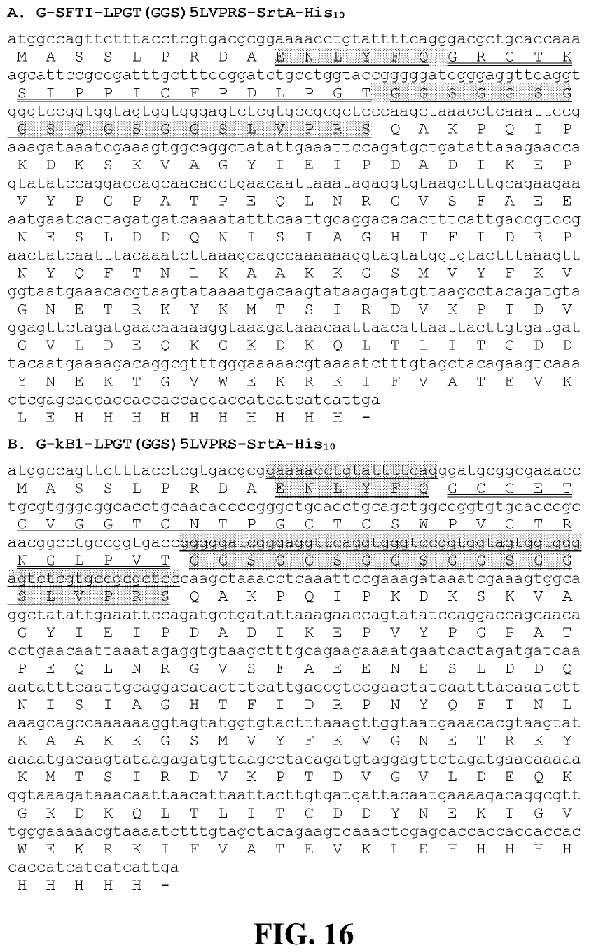

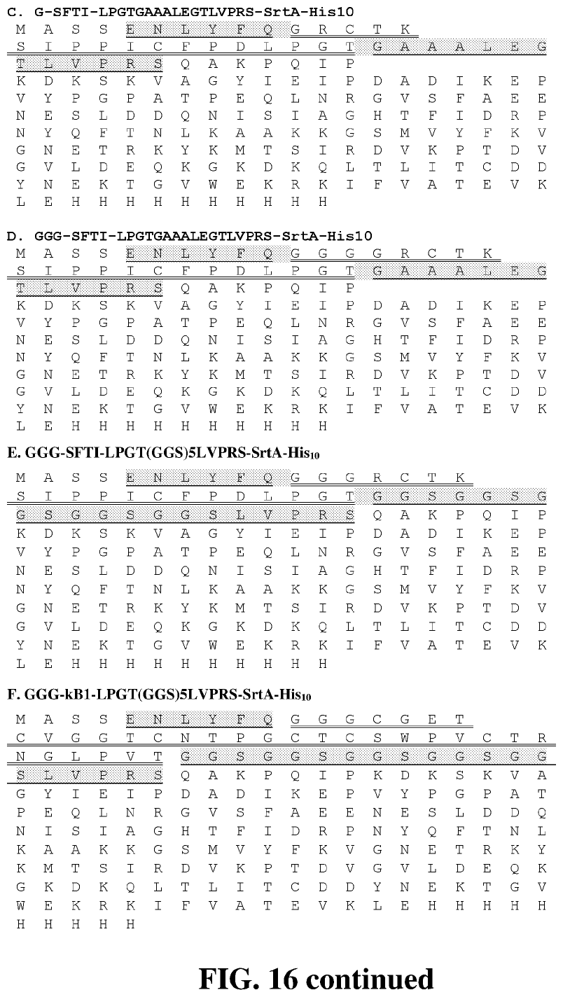

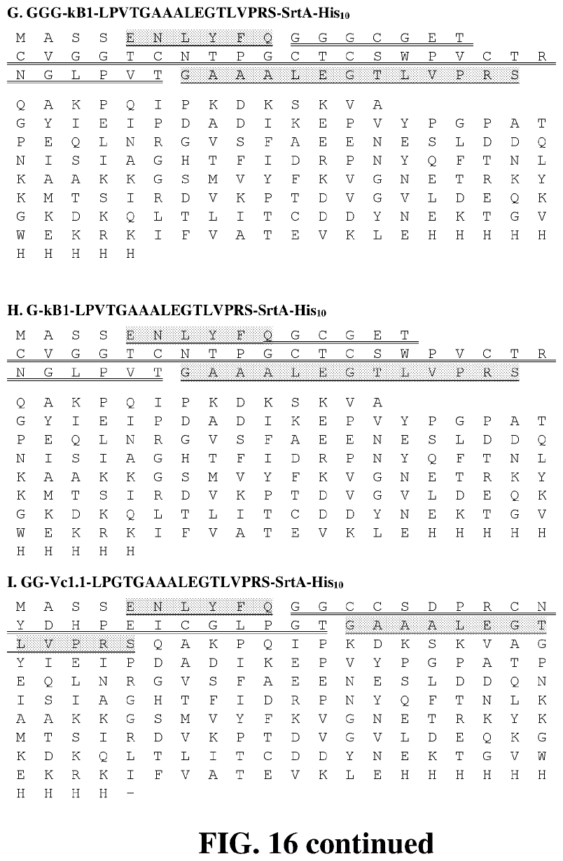

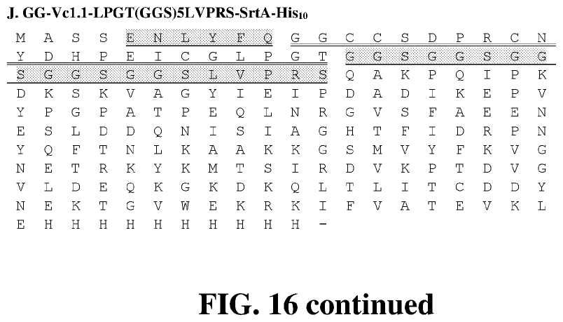

BRIEF DESCRIPTION OF THE SEQUENCES

| SEQ | | |
|---|---|---|
| ID | | |
| NO: | Description | Sequence |
| 1 | His₆-TEV-MSP9-eSrtA | MGSSHHHHHHENLYFQGSTFSKLREQLGPVTQEFWDNLE |
| | amino acid sequence, | KETEGLRQEMSKDLEEVKAKVQPYLDDFQKKWQEEMELY |
| | shown in FIG. 7. | RQKVEPLGEEMRDRARAHVDALRTHLAPYSDELRQRLAA |
| | | RLEALKENGGARLAEYHAKATEHLSTLSEKAKPALEDLRQ |
| | | GLLPVLESFKVSFLSALEEYTKKLNTQLPGTGAAALEGTQA |
| | | KPQIPKDKSKVAGYIEIPDADIKEPVYPGPATREQLNRGVSF |
| | | AEENESLDDQNISIAGHTFIDRPNYQFTNLKAAKKGSMVYF |
| | | KVGNETRKYKMTSIRNVKPTAVEVLDEQKGKDKQLTLITC |
| | | DDYNEETGVWETRKIFVATEVKLEHHHHHH |
| 2 | MSP9-LPGT(GGS)x5- | MASSENLYFQGSTFSKLREQLGPVTQEFWDNLEKETEGLR |
| | SrtA-His₁₀ amino acid | QEMSKDLEEVKAKVQPYLDDFQKKWQEEMELYRQKVEP |
| | sequence, shown in FIG. | LGEEMRDRARAHVDALRTHLAPYSDELRQRLAARLEALK |
| | 14A. | ENGGARLAEYHAKATEHLSTLSEKAKPALEDLRQGLLPVL |
| | | ESFKVSFLSALEEYTKKLNTQLPGTGGSGGSGGSGGSGGSQ |
| | | AKPQIPKDKSKVAGYIEIPDADIKEPVYPGPATPEQLNRGVS |
| | | FAEENESLDDQNISIAGHTFIDRPNYQFTNLKAAKKGSMVY |
| | | FKVGNETRKYKMTSIRDVKPTDVGVLDEQKGKDKQLTLIT |
| | | CDDYNEKTGVWEKRKIFVATEVKLEHHHHHHHHHH |
| 3 | MSP11-LPGT(GGS)5- | MASSENLYFQGSTFSKLREQLGPVTQEFWDNLEKETEGLR |
| | wtSrtA-His₁₀ amino acid | QEMSKDLEEVKAKVQPYLDDFQKKWQEEMELYRQKVEP |
| | sequence, shown in FIG. | LRAELQEGARQKLHELQEKLSPLGEEMRDRARAHVDALR |
| | 14B. | THLAPYSDELRQRLAARLEALKENGGARLAEYHAKATEH |
| | | LSTLSEKAKPALEDLRQGLLPVLESFKVSFLSALEEYTKKL |
| | | NTQLPGTGGSGGSGGSGGSGGSQAKPQIPKDKSKVAGYIEI |
| | | PDADIKEPVYPGPATPEQLNRGVSFAEENESLDDQNISIAG |
| | | HTFIDRPNYQFTNLKAAKKGSMVYFKVGNETRKYKMTSIR |
| | | DVKPTDVGVLDEQKGKDKQLTLITCDDYNEKTGVWEKRK |
| | | IFVATEVKLEHHHHHHHHHH |
| 4 | MSP20-LPGT(GGS)5- | MASSENLYFQGSTFSKLREQLGPVTQEFWDNLEKETEGLR |
| | wtSrtA-His₁₀ amino acid | QEMSKDLEEVKAKVQPYLDDFQKKWQEEMELYRQKVEP |
| | sequence, shown in FIG. | LRAELQEGARQKLHELQEKLSPLGEEMRDRARAHVDALR |
| | 14C. | THLAPYSDELRQRLAARLEALKENGGARLAEYHAKATEH |
| | | LSTLSEKAKPALEDLRQGLLPVLESFKVSFLSALEEYTKKL |
| | | NTQGTPVTQEFWDNLEKETEGLRQEMSKDLEEVKAKVQP |
| | | YLDDFQKKWQEEMELYRQKVEPLRAELQEGARQKLHELQ |
| | | EKLSPLGEEMRDRARAHVDALRTHLAPYSDELRQRLAARL |
| | | EALKENGGARLAEYHAKATEHLSTLSEKAKPALEDLRQGL |
| | | LPVLESFKVSFLSALEEYTKKLNTQLPGTGGSGGSGGSGGS |
| | | GGSQAKPQIPKDKSKVAGYIEIPDADIKEPVYPGPATPEQL |
| | | NRGVSFAEENESLDDQNISIAGHTFIDRPNYQFTNLKAAKK |
| | | GSMVYFKVGNETRKYKMTSIRDVKPTDVGVLDEQKGKDK |
| | | QLTLITCDDYNEKTGVWEKRKIFVATEVKLEHHHHHHHH |
| | | HH |
| 5 | MSP7-LPGT(GGS)5- | MASSENLYFQGSTFSKLREQLGPVTQEFWDNLEKETEGLR |
| | wtSrtA-His₁₀ amino acid | QEMSKDLEEVKAKVQPLGEEMRDRARAHVDALRTHLAPY |
| | sequence, shown in FIG. | SDELRQRLAARLEALKENGGARLAEYHAKATEHLSTLSEK |
| | 14D. | AKPALEDLRQGLLPVLESFKVSFLSALEEYTKKLNTQLPGT |
| | | GGSGGSGGSGGSGGSQAKPQIPKDKSKVAGYIEIPDADIKE |
| | | PVYPGPATPEQLNRGVSFAEENESLDDQNISIAGHTFIDRPN |
| | | YQFTNLKAAKKGSMVYFKVGNETRKYKMTSIRDVKPTDV |
| | | GVLDEQKGKDKQLTLITCDDYNEKTGVWEKRKIFVATEV |
| | | KLEHHHHHHHHHH |
| 6 | MSP6-LPGT(GGS)5- | MASSENLYFQGSTFSKLREQLGPVTQEFWDNLEKETEGLR |
| | wtSrtA-His₁₀ amino acid | QEMSKDLEEVKAKVQPYSDELRQRLAARLEALKENGGAR |
| | sequence, shown in FIG. | LAEYHAKATEHLSTLSEKAKPALEDLRQGLLPVLESFKVSF |
| | 14E. | LSALEEYTKKLNTQLPGTGGSGGSGGSGGSGGSQAKPQIP |
| | | KDKSKVAGYIEIPDADIKEPVYPGPATPEQLNRGVSFAEEN |
| | | ESLDDQNISIAGHTFIDRPNYQFTNLKAAKKGSMVYFKVG |
| | | NETRKYKMTSIRDVKPTDVGVLDEQKGKDKQLTLITCDDY |
| | | NEKTGVWEKRKIFVATEVKLEHHHHHHHHHH |
| 7 | MSP9- | MASSENLYFQGSTFSKLREQLGPVTQEFWDNLEKETEGLR |
| | LPGTGAAALEGTLVPR | QEMSKDLEEVKAKVQPYLDDFQKKWQEEMELYRQKVEP |
| | S-SrtA-His₁₀ amino acid | LGEEMRDRARAHVDALRTHLAPYSDELRQRLAARLEALK |
| | sequence, shown in FIG. | ENGGARLAEYHAKATEHLSTLSEKAKPALEDLRQGLLPVL |
| | 15A. | ESFKVSFLSALEEYTKKLNTQLPGTGAAALEGTLVPRSQAK |
| | | PQIPKDKSKVAGYIEIPDADIKEPVYPGPATPEQLNRGVSFA |
| | | EENESLDDQNISIAGHTFIDRPNYQFTNLKAAKKGSMVYFK |
| | | VGNETRKYKMTSIRDVKPTDVGVLDEQKGKDKQLTLITCD |
| | | DYNEKTGVWEKRKIFVATEVKLEHHHHHHHHHH |
| 8 | MSP7- | MASSENLYFQGSTFSKLREQLGPVTQEFWDNLEKETEGLR |
| | LPGTGAAALEGTLVPR | QEMSKDLEEVKAKVQPLGEEMRDRARAHVDALRTHLAPY |
| | S-SrtA-His₁₀ amino acid | SDELRQRLAARLEALKENGGARLAEYHAKATEHLSTLSEK |
| | sequence, shown in FIG. | AKPALEDLRQGLLPVLESFKVSFLSALEEYTKKLNTQLPGT |
| | 15B. | GAAALEGTLVPRSQAKPQIPKDKSKVAGYIEIPDADIKEPV |
| | | YPGPATPEQLNRGVSFAEENESLDDQNISIAGHTFIDRPNYQ |
| | | FTNLKAAKKGSMVYFKVGNETRKYKMTSIRDVKPTDVGV |
| | | LDEQKGKDKQLTLITCDDYNEKTGVWEKRKIFVATEVKLE |
| | | HHHHHHHHHH |
| 9 | MSP6- | MASSENLYFQGSTFSKLREQLGPVTQEFWDNLEKETEGLR |
| | LPGTGAAALEGTLVPR | QEMSKDLEEVKAKVQPYSDELRQRLAARLEALKENGGAR |
| | S-SrtA-His₁₀ amino acid | LAEYHAKATEHLSTLSEKAKPALEDLRQGLLPVLESFKVSF |
| | sequence, shown in FIG. | LSALEEYTKKLNTQLPGTGAAALEGTLVPRSQAKPQIPKDK |
| | 15C. | SKVAGYIEIPDADIKEPVYPGPATPEQLNRGVSFAEENESLD |
| | | DQNISIAGHTFIDRPNYQFTNLKAAKKGSMVYFKVGNETR |
| | | KYKMTSIRDVKPTDVGVLDEQKGKDKQLTLITCDDYNEKT |
| | | GVWEKRKIFVATEVKLEHHHHHHHHHH |
| 10 | MSP11- | MASSENLYFQGSTFSKLREQLGPVTQEFWDNLEKETEGLR |
| | LPGTGAAALEGTLVPR | QEMSKDLEEVKAKVQPYLDDFQKKWQEEMELYRQKVEP |
| | S-SrtA-His₁₀ amino acid | LRAELQEGARQKLHELQEKLSPLGEEMRDRARAHVDALR |
| | sequence, shown in FIG. | THLAPYSDELRQRLAARLEALKENGGARLAEYHAKATEH |
| | 15D. | LSTLSEKAKPALEDLRQGLLPVLESFKVSFLSALEEYTKKL |
| | | NTQLPGTGAAALEGTLVPRSQAKPQIPKDKSKVAGYIEIPD |
| | | ADIKEPVYPGPATPEQLNRGVSFAEENESLDDQNISIAGHTF |
| | | IDRPNYQFTNLKAAKKGSMVYFKVGNETRKYKMTSIRDV |
| | | KPTDVGVLDEQKGKDKQLTLITCDDYNEKTGVWEKRKIF |
| | | VATEVKLEHHHHHHHHHH |
| 11 | MSP20- | MASSENLYFQGSTFSKLREQLGPVTQEFWDNLEKETEGLR |
| | LPGTGAAALEGTLVPR | QEMSKDLEEVKAKVQPYLDDFQKKWQEEMELYRQKVEP |
| | S-SrtA-His₁₀ amino acid | LRAELQEGARQKLHELQEKLSPLGEEMRDRARAHVDALR |
| | sequence, shown in FIG. | THLAPYSDELRQRLAARLEALKENGGARLAEYHAKATEH |
| | 15E. | LSTLSEKAKPALEDLRQGLLPVLESFKVSFLSALEEYTKKL |
| | | NTQGTPVTQEFWDNLEKETEGLRQEMSKDLEEVKAKVQP |
| | | YLDDFQKKWQEEMELYRQKVEPLRAELQEGARQKLHELQ |
| | | EKLSPLGEEMRDRARAHVDALRTHLAPYSDELRQRLAARL |
| | | EALKENGGARLAEYHAKATEHLSTLSEKAKPALEDLRQGL |
| | | LPVLESFKVSFLSALEEYTKKLNTQLPGTGAAALEGTLVPR |
| | | SQAKPQIPKDKSKVAGYIEIPDADIKEPVYPGPATPEQLNRG |
| | | VSFAEENESLDDQNISIAGHTFIDRPNYQFTNLKAAKKGSM |
| | | VYFKVGNETRKYKMTSIRDVKPTDVGVLDEQKGKDKQLT |
| | | LITCDDYNEKTGVWEKRKIFVATEVKLEHHHHHHHHHH |
| 12 | G-SFTI- | MASSLPRDAENLYFQGRCTKSIPPICFPDLPGTGGSGGSGGS |
| | LPGT(GGS)5LVPRS- | GGSGGSLVPRSQAKPQIPKDKSKVAGYIEIPDADIKEPVYP |
| | SrtA-His₁₀ amino acid | GPATPEQLNRGVSFAEENESLDDQNISIAGHTFIDRPNYQFT |
| | sequence, shown in FIG. | NLKAAKKGSMVYFKVGNETRKYKMTSIRDVKPTDVGVLD |
| | 16A. | EQKGKDKQLTLITCDDYNEKTGVWEKRKIFVATEVKLEHH |
| | | HHHHHHHH |
| 13 | G-kB1- | MASSLPRDAENLYFQGCGETCVGGTCNTPGCTCSWPVCTR |
| | LPGT(GGS)5LVPRS- | NGLPVTGGSGGSGGSGGSGGSLVPRSQAKPQIPKDKSKVA |
| | SrtA-His₁₀ amino acid | GYIEIPDADIKEPVYPGPATPEQLNRGVSFAEENESLDDQNI |
| | sequence, shown in FIG. | SIAGHTFIDRPNYQFTNLKAAKKGSMVYFKVGNETRKYK |
| | 16B. | MTSIRDVKPTDVGVLDEQKGKDKQLTLITCDDYNEKTGV |
| | | WEKRKIFVATEVKLEHHHHHHHHHH |
| 14 | G-SFTI- | MASSENLYFQGRCTKSIPPICFPDLPGTGAAALEGTLVPRSQ |
| | LPGTGAAALEGTLVPR | AKPQIPKDKSKVAGYIEIPDADIKEPVYPGPATPEQLNRGVS |
| | S-SrtA-His₁₀ amino acid | FAEENESLDDQNISIAGHTFIDRPNYQFTNLKAAKKGSMVY |
| | sequence of FIG. 16C. | FKVGNETRKYKMTSIRDVKPTDVGVLDEQKGKDKQLTLIT |
| | | CDDYNEKTGVWEKRKIFVATEVKLEHHHHHHHHHH |
| 15 | GGG-SFTI- | MASSENLYFQGGGGRCTKSIPPICFPDLPGTGAAALEGTLV |
| | LPGTGAAALEGTLVPR | PRSQAKPQIPKDKSKVAGYIEIPDADIKEPVYPGPATPEQLN |
| | S-SrtA-His₁₀ amino acid | RGVSFAEENESLDDQNISIAGHTFIDRPNYQFTNLKAAKKG |
| | sequence of FIG. 16D. | SMVYFKVGNETRKYKMTSIRDVKPTDVGVLDEQKGKDKQ |
| | | LTLITCDDYNEKTGVWEKRKIFVATEVKLEHHHHHHHHH |
| | | H |
| 16 | GGG-SFTI- | MASSENLYFQGGGRCTKSIPPICFPDLPGTGGSGGSGGSGG |
| | LPGT(GGS)5LVPRS- | SGGSLVPRSQAKPQIPKDKSKVAGYIEIPDADIKEPVYPGPA |
| | SrtA-His₁₀ amino acid | TPEQLNRGVSFAEENESLDDQNISIAGHTFIDRPNYQFTNLK |
| | sequence, shown in FIG. | AAKKGSMVYFKVGNETRKYKMTSIRDVKPTDVGVLDEQK |
| | 16E. | GKDKQLTLITCDDYNEKTGVWEKRKIFVATEVKLEHHHH |
| | | HHHHHH |
| 17 | GGG-kB1- | MASSENLYFQGGGCGETCVGGTCNTPGCTCSWPVCTRNG |
| | LPGT(GGS)5LVPRS- | LPVTGGSGGSGGSGGSGGSLVPRSQAKPQIPKDKSKVAGYI |
| | SrtA-His₁₀ amino acid | EIPDADIKEPVYPGPATPEQLNRGVSFAEENESLDDQNISIA |
| | sequence, shown in FIG. | GHTFIDRPNYQFTNLKAAKKGSMVYFKVGNETRKYKMTSI |
| | 16F. | RDVKPTDVGVLDEQKGKDKQLTLITCDDYNEKTGVWEKR |
| | | KIFVATEVKLEHHHHHHHHHH |
| 18 | GGG-kB1- | MASSENLYFQGGGCGETCVGGTCNTPGCTCSWPVCTRNG |
| | LPVTGAAALEGTLVPR | LPVTGAAALEGTLVPRSQAKPQIPKDKSKVAGYIEIPDADI |
| | S-SrtA-His₁₀ amino acid | KEPVYPGPATPEQLNRGVSFAEENESLDDQNISIAGHTFIDR |
| | sequence, shown in FIG. | PNYQFTNLKAAKKGSMVYFKVGNETRKYKMTSIRDVKPT |
| | 16G. | DVGVLDEQKGKDKQLTLITCDDYNEKTGVWEKRKIFVAT |
| | | EVKLEHHHHHHHHHH |
| 19 | G-kB1- | MASSENLYFQGCGETCVGGTCNTPGCTCSWPVCTRNGLP |
| | LPVTGAAALEGTLVPR | VTGAAALEGTLVPRSQAKPQIPKDKSKVAGYIEIPDADIKE |
| | S-SrtA-His₁₀ amino acid | PVYPGPATPEQLNRGVSFAEENESLDDQNISIAGHTFIDRPN |
| | sequence, shown in FIG. | YQFTNLKAAKKGSMVYFKVGNETRKYKMTSIRDVKPTDV |
| | 16H. | GVLDEQKGKDKQLTLITCDDYNEKTGVWEKRKIFVATEV |
| | | KLEHHHHHHHHHH |
| 20 | GG-Vc1.1- | MASSENLYFQGGCCSDPRCNYDHPEICGLPGTGAAALEGT |
| | LPGTGAAALEGTLVPR | LVPRSQAKPQIPKDKSKVAGYIEIPDADIKEPVYPGPATPEQ |
| | S-SrtA-His₁₀ amino acid | LNRGVSFAEENESLDDQNISIAGHTFIDRPNYQFTNLKAAK |
| | sequence, shown in FIG. | KGSMVYFKVGNETRKYKMTSIRDVKPTDVGVLDEQKGKD |
| | 16I. | KQLTLITCDDYNEKTGVWEKRKIFVATEVKLEHHHHHHH |
| | | HHH |
| 21 | GG-Vc1.1- | MASSENLYFQGGCCSDPRCNYDHPEICGLPGTGGSGGSGG |
| | LPGT(GGS)5LVPRS- | SGGSGGSLVPRSQAKPQIPKDKSKVAGYIEIPDADIKEPVYP |
| | SrtA-His₁₀ amino acid | GPATPEQLNRGVSFAEENESLDDQNISIAGHTFIDRPNYQFT |
| | sequence, shown in FIG. | NLKAAKKGSMVYFKVGNETRKYKMTSIRDVKPTDVGVLD |
| | 16J. | EQKGKDKQLTLITCDDYNEKTGVWEKRKIFVATEVKLEHH |
| | | HHHHHHHH |
| 22 | His₆-TEV-MSP9-eSrtA | ATGGGGTCGTCCCACCATCACCACCATCATGAGAATTTG |
| | nucleotide sequence, | TACTTCCAAGGATCGACGTTTTCCAAGTTACGCGAACAG |
| | shown in FIG. 7. | TTAGGACCCGTAACGCAGGAATTCTGGGACAACCTTGA |
| | | GAAAGAGACGGAAGGCCTTCGCCAGGAGATGTCAAAAG |
| | | ACCTTGAGGAAGTGAAGGCTAAGGTACAACCCTATCTG |
| | | GACGATTTTCAAAAGAAGTGGCAAGAAGAAATGGAGTT |
| | | GTATCGTCAAAAAGTTGAACCTTTGGGGGAGGAGATGC |
| | | GTGATCGCGCCCGCGCCCACGTGGATGCATTGCGCACGC |
| | | ATTTAGCTCCATATAGTGATGAGTTGCGCCAGCGTTTGG |
| | | CCGCACGTTTAGAGGCTTTGAAAGAGAATGGCGGTGCC |
| | | CGTCTGGCCGAGTACCATGCAAAGGCGACAGAACATTT |
| | | GTCCACCTTGAGCGAGAAAGCTAAACCGGCTCTGGAGG |
| | | ACTTGCGTCAGGGCTTGCTTCCGGTACTTGAATCATTCA |
| | | AGGTGTCCTTTCTGTCTGCCTTAGAAGAGTATACTAAGA |
| | | AGCTTAACACACAACTGCCTGGCACAGGTGCTGCAGCTT |
| | | TAGAGGGTACCCAAGCTAAACCGCAGATCCCCAAAGAC |
| | | AAATCTAAAGTTGCAGGTTATATTGAGATCCCAGACGCG |
| | | GATATTAAGGAGCCCGTGTATCCGGGTCCCGCCACTCGC |
| | | GAGCAGTTGAATCGCGGAGTCTCCTTTGCAGAGGAAAA |
| | | TGAATCGTTGGATGACCAGAATATCTCTATTGCCGGTCA |
| | | TACATTCATCGACCGTCCAAATTACCAATTCACTAACCT |
| | | TAAAGCCGCGAAAAAGGGGTCGATGGTCTATTTCAAGG |
| | | TGGGCAATGAAACACGCAAATATAAAATGACTTCGATT |
| | | CGTAACGTCAAACCAACGGCTGTGGAAGTGTTAGACGA |
| | | GCAAAAAGGCAAGGATAAGCAACTTACTTTAATTACGT |
| | | GTGACGATTATAATGAAGAGACAGGAGTATGGGAGACA |
| | | CGCAAAATCTTCGTGGCGACGGAGGTTAAGCTCGAGCA |
| | | TCATCATCATCACCACTAG |
| 23 | MSP9-LPGT(GGS)x5- | ATGGCTTCGTCCGAGAATTTGTACTTCCAAGGATCGACG |
| | SrtA-His₁₀ nucleotide | TTTTCCAAGTTACGCGAACAGTTAGGACCCGTAACGCAG |
| | sequence, shown in FIG. | GAATTCTGGGACAACCTTGAGAAAGAGACGGAAGGCCT |
| | 14A. | TCGCCAGGAGATGTCAAAAGACCTTGAGGAAGTGAAGG |
| | | CTAAGGTACAACCCTATCTGGACGATTTTCAAAAGAAGT |
| | | GGCAAGAAGAAATGGAGTTGTATCGTCAAAAAGTTGAA |
| | | CCTTTGGGGGAGGAGATGCGTGATCGCGCCCGCGCCCA |
| | | CGTGGATGCATTGCGCACGCATTTAGCTCCATATAGTGA |
| | | TGAGTTGCGCCAGCGTTTGGCCGCACGTTTAGAGGCTTT |
| | | GAAAGAGAATGGCGGTGCCCGTCTGGCCGAGTACCATG |
| | | CAAAGGCGACAGAACATTTGTCCACCTTGAGCGAGAAA |
| | | GCTAAACCGGCTCTGGAGGACTTGCGTCAGGGCTTGCTT |
| | | CCGGTACTTGAATCATTCAAGGTGTCCTTTCTGTCTGCCT |
| | | TAGAAGAGTATACTAAGAAGCTTAACACACAACTGCCT |
| | | GGTACCGGGGGATCGGGAGGTTCAGGTGGGTCCGGTGG |
| | | TAGTGGTGGGAGTCAAGCTAAACCTCAAATTCCGAAAG |
| | | ATAAATCGAAAGTGGCAGGCTATATTGAAATTCCAGAT |
| | | GCTGATATTAAAGAACCAGTATATCCAGGACCAGCAAC |
| | | ACCTGAACAATTAAATAGAGGTGTAAGCTTTGCAGAAG |
| | | AAAATGAATCACTAGATGATCAAAATATTTCAATTGCAG |
| | | GACACACTTTCATTGACCGTCCGAACTATCAATTTACAA |
| | | ATCTTAAAGCAGCCAAAAAAGGTAGTATGGTGTACTTTA |
| | | AAGTTGGTAATGAAACACGTAAGTATAAAATGACAAGT |
| | | ATAAGAGATGTTAAGCCTACAGATGTAGGAGTTCTAGAT |
| | | GAACAAAAAGGTAAAGATAAACAATTAACATTAATTAC |
| | | TTGTGATGATTACAATGAAAAGACAGGCGTTTGGGAAA |
| | | AACGTAAAATCTTTGTAGCTACAGAAGTCAAACTCGAGC |
| | | ACCACCACCACCACCACCATCATCATCATTGA |
| 24 | MSP11-LPGT(GGS)5- | ATGGCTAGCAGCGAAAACCTGTATTTTCAGGGCAGCACC |
| | wtSrtA-His₁₀ nucleotide | TTTAGCAAACTGCGTGAACAGCTGGGCCCGGTGACCCA |
| | sequence, shown in FIG. | GGAATTTTGGGATAACCTGGAAAAAGAAACCGAAGGCC |
| | 14B. | TGCGTCAGGAAATGAGCAAAGATCTGGAAGAGGTGAAA |
| | | GCGAAAGTGCAGCCGTATCTGGATGACTTTCAGAAAAA |
| | | ATGGCAGGAAGAGATGGAACTGTATCGTCAGAAAGTGG |
| | | AACCGCTGCGTGCGGAACTGCAGGAAGGCGCGCGTCAG |
| | | AAACTGCATGAACTGCAGGAAAAACTGAGCCCGCTGGG |
| | | CGAAGAGATGCGTGATCGTGCGCGTGCGCATGTGGATG |
| | | CGCTGCGTACCCATCTGGCGCCGTATAGCGATGAACTGC |
| | | GTCAGCGTCTGGCGGCCCGTCTGGAAGCGCTGAAAGAA |
| | | AACGGCGGTGCGCGTCTGGCGGAATATCATGCGAAAGC |
| | | GACCGAACATCTGAGCACCCTGAGCGAAAAAGCGAAAC |
| | | CGGCGCTGGAAGATCTGCGTCAGGGCCTGCTGCCGGTG |
| | | CTGGAAAGCTTTAAAGTGAGCTTTCTGAGCGCGCTGGAA |
| | | GAGTATACCAAAAAACTGAACACCCAGCTGCCGGGTAC |
| | | CGGGGGATCGGGAGGTTCAGGTGGGTCCGGTGGTAGTG |
| | | GTGGGAGTCAAGCTAAACCTCAAATTCCGAAAGATAAA |
| | | TCGAAAGTGGCAGGCTATATTGAAATTCCAGATGCTGAT |
| | | ATTAAAGAACCAGTATATCCAGGACCAGCAACACCTGA |
| | | ACAATTAAATAGAGGTGTAAGCTTTGCAGAAGAAAATG |
| | | AATCACTAGATGATCAAAATATTTCAATTGCAGGACACA |
| | | CTTTCATTGACCGTCCGAACTATCAATTTACAAATCTTA |
| | | AAGCAGCCAAAAAAGGTAGTATGGTGTACTTTAAAGTT |
| | | GGTAATGAAACACGTAAGTATAAAATGACAAGTATAAG |
| | | AGATGTTAAGCCTACAGATGTAGGAGTTCTAGATGAAC |
| | | AAAAAGGTAAAGATAAACAATTAACATTAATTACTTGT |
| | | GATGATTACAATGAAAAGACAGGCGTTTGGGAAAAACG |
| | | TAAAATCTTTGTAGCTACAGAAGTCAAACTCGAGCACCA |
| | | CCACCACCACCACCATCATCATCATTGA |
| 25 | MSP20-LPGT(GGS)5- | ATGGCATCGTCGGAGAACTTGTATTTCCAAGGCTCTACT |
| | wtSrtA-His₁₀ nucleotide | TTCTCGAAGTTGCGTGAGCAGTTGGGACCTGTGACACAA |
| | sequence, shown in FIG. | GAGTTCTGGGATAATTTAGAAAAGGAGACAGAAGGGCT |
| | 14C. | GCGTCAAGAGATGAGTAAAGACCTTGAAGAAGTTAAAG |
| | | CAAAGGTGCAGCCCTATCTGGATGATTTCCAAAAAAAAT |
| | | GGCAAGAAGAAATGGAATTATACCGTCAGAAGGTAGAG |
| | | CCACTTCGTGCAGAATTGCAAGAAGGCGCACGCCAGAA |
| | | GTTGCACGAACTGCAAGAAAAATTGTCACCTTTGGGGG |
| | | AGGAGATGCGCGACCGTGCACGCGCGCACGTTGACGCC |
| | | TTGCGTACGCATCTGGCGCCGTACTCTGACGAATTACGT |
| | | CAGCGCTTGGCCGCGCGCTTAGAGGCCTTGAAGGAGAA |
| | | CGGGGGAGCGCGTCTTGCAGAGTACCATGCCAAAGCCA |
| | | CGGAACATCTGTCCACCTTGAGCGAGAAGGCGAAGCCA |
| | | GCACTGGAAGACTTACGCCAGGGTTTGCTGCCAGTCCTT |
| | | GAGTCTTTTAAAGTATCGTTTCTTTCTGCGCTTGAGGAAT |
| | | ACACGAAGAAGTTAAACACTCAGGGTACTCCAGTGACA |
| | | CAGGAGTTTTGGGATAATTTGGAAAAAGAGACTGAAGG |
| | | GCTTCGCCAAGAGATGTCGAAGGATTTGGAAGAGGTAA |
| | | AGGCGAAGGTCCAACCTTACCTGGACGATTTCCAAAAG |
| | | AAGTGGCAGGAAGAAATGGAGTTATACCGTCAGAAAGT |
| | | CGAACCTTTACGTGCCGAATTACAAGAAGGAGCACGCC |
| | | AAAAACTTCATGAGCTTCAGGAGAAGCTGTCCCCCCTTG |
| | | GTGAGGAGATGCGCGACCGTGCGCGTGCTCATGTAGAT |
| | | GCATTACGTACCCACCTTGCCCCCTATAGCGATGAGTTG |
| | | CGTCAGCGTCTTGCCGCCCGCCTGGAAGCTTTGAAAGAG |
| | | AATGGCGGTGCTCGTTTAGCAGAGTATCACGCCAAGGCC |
| | | ACCGAACATCTTTCAACTTTGTCTGAGAAAGCCAAACCT |
| | | GCGTTAGAAGACTTGCGTCAAGGGCTTCTGCCTGTCTTA |
| | | GAGTCGTTCAAGGTGTCATTTCTGTCGGCGCTTGAAGAA |
| | | TATACTAAAAAGTTGAATACACAGTTACCTGGTACCGGG |
| | | GGATCGGGAGGTTCAGGTGGGTCCGGTGGTAGTGGTGG |
| | | GAGTCAAGCTAAACCTCAAATTCCGAAAGATAAATCGA |
| | | AAGTGGCAGGCTATATTGAAATTCCAGATGCTGATATTA |
| | | AAGAACCAGTATATCCAGGACCAGCAACACCTGAACAA |
| | | TTAAATAGAGGTGTAAGCTTTGCAGAAGAAAATGAATC |
| | | ACTAGATGATCAAAATATTTCAATTGCAGGACACACTTT |
| | | CATTGACCGTCCGAACTATCAATTTACAAATCTTAAAGC |
| | | AGCCAAAAAAGGTAGTATGGTGTACTTTAAAGTTGGTA |
| | | ATGAAACACGTAAGTATAAAATGACAAGTATAAGAGAT |
| | | GTTAAGCCTACAGATGTAGGAGTTCTAGATGAACAAAA |
| | | AGGTAAAGATAAACAATTAACATTAATTACTTGTGATGA |
| | | TTACAATGAAAAGACAGGCGTTTGGGAAAAACGTAAAA |
| | | TCTTTGTAGCTACAGAAGTCAAACTCGAGCACCACCACC |
| | | ACCACCACCATCATCATCATTGA |
| 26 | MSP7-LPGT(GGS)5- | ATGGCTTCGTCCGAGAATTTGTACTTCCAAGGATCGACG |
| | wtSrtA-His₁₀ nucleotide | TTTTCCAAGTTACGCGAACAGTTAGGACCCGTAACGCAG |
| | sequence, shown in FIG. | GAATTCTGGGACAACCTTGAGAAAGAGACGGAAGGCCT |
| | 14D. | TCGCCAGGAGATGTCAAAAGACCTTGAGGAAGTGAAGG |
| | | CTAAGGTACAACCCTTGGGGGAGGAGATGCGTGATCGC |
| | | GCCCGCGCCCACGTGGATGCATTGCGCACGCATTTAGCT |
| | | CCATATAGTGATGAGTTGCGCCAGCGTTTGGCCGCACGT |
| | | TTAGAGGCTTTGAAAGAGAATGGCGGTGCCCGTCTGGCC |
| | | GAGTACCATGCAAAGGCGACAGAACATTTGTCCACCTTG |
| | | AGCGAGAAAGCTAAACCGGCTCTGGAGGACTTGCGTCA |
| | | GGGCTTGCTTCCGGTACTTGAATCATTCAAGGTGTCCTTT |
| | | CTGTCTGCCTTAGAAGAGTATACTAAGAAGCTTAACACA |
| | | CAACTGCCTGGTACCGGGGGATCGGGAGGTTCAGGTGG |
| | | GTCCGGTGGTAGTGGTGGGAGTCAAGCTAAACCTCAAA |
| | | TTCCGAAAGATAAATCGAAAGTGGCAGGCTATATTGAA |
| | | ATTCCAGATGCTGATATTAAAGAACCAGTATATCCAGGA |
| | | CCAGCAACACCTGAACAATTAAATAGAGGTGTAAGCTTT |
| | | GCAGAAGAAAATGAATCACTAGATGATCAAAATATTTC |
| | | AATTGCAGGACACACTTTCATTGACCGTCCGAACTATCA |
| | | ATTTACAAATCTTAAAGCAGCCAAAAAAGGTAGTATGG |
| | | TGTACTTTAAAGTTGGTAATGAAACACGTAAGTATAAAA |
| | | TGACAAGTATAAGAGATGTTAAGCCTACAGATGTAGGA |
| | | GTTCTAGATGAACAAAAAGGTAAAGATAAACAATTAAC |
| | | ATTAATTACTTGTGATGATTACAATGAAAAGACAGGCGT |
| | | TTGGGAAAAACGTAAAATCTTTGTAGCTACAGAAGTCA |
| | | AACTCGAGCACCACCACCACCACCACCATCATCATCATT |
| | | GA |
| 27 | MSP6-LPGT(GGS)5- | ATGGCTTCGTCCGAGAATTTGTACTTCCAAGGATCGACG |
| | wtSrtA-His₁₀ nucleotide | TTTTCCAAGTTACGCGAACAGTTAGGACCCGTAACGCAG |
| | sequence, shown in FIG. | GAATTCTGGGACAACCTTGAGAAAGAGACGGAAGGCCT |
| | 14E. | TCGCCAGGAGATGTCAAAAGACCTTGAGGAAGTGAAGG |
| | | CTAAGGTACAACCCTATAGTGATGAGTTGCGCCAGCGTT |
| | | TGGCCGCACGTTTAGAGGCTTTGAAAGAGAATGGCGGT |
| | | GCCCGTCTGGCCGAGTACCATGCAAAGGCGACAGAACA |
| | | TTTGTCCACCTTGAGCGAGAAAGCTAAACCGGCTCTGGA |
| | | GGACTTGCGTCAGGGCTTGCTTCCGGTACTTGAATCATT |
| | | CAAGGTGTCCTTTCTGTCTGCCTTAGAAGAGTATACTAA |
| | | GAAGCTTAACACACAACTGCCTGGTACCGGGGGATCGG |
| | | GAGGTTCAGGTGGGTCCGGTGGTAGTGGTGGGAGTCAA |
| | | GCTAAACCTCAAATTCCGAAAGATAAATCGAAAGTGGC |
| | | AGGCTATATTGAAATTCCAGATGCTGATATTAAAGAACC |
| | | AGTATATCCAGGACCAGCAACACCTGAACAATTAAATA |
| | | GAGGTGTAAGCTTTGCAGAAGAAAATGAATCACTAGAT |
| | | GATCAAAATATTTCAATTGCAGGACACACTTTCATTGAC |
| | | CGTCCGAACTATCAATTTACAAATCTTAAAGCAGCCAAA |
| | | AAAGGTAGTATGGTGTACTTTAAAGTTGGTAATGAAACA |
| | | CGTAAGTATAAAATGACAAGTATAAGAGATGTTAAGCC |
| | | TACAGATGTAGGAGTTCTAGATGAACAAAAAGGTAAAG |
| | | ATAAACAATTAACATTAATTACTTGTGATGATTACAATG |
| | | AAAAGACAGGCGTTTGGGAAAAACGTAAAATCTTTGTA |
| | | GCTACAGAAGTCAAACTCGAGCACCACCACCACCACCA |
| | | CCATCATCATCATTGA |
| 28 | MSP9- | ATGGCTTCGTCCGAGAATTTGTACTTCCAAGGATCGACG |
| | LPGTGAAALEGTLVPR | TTTTCCAAGTTACGCGAACAGTTAGGACCCGTAACGCAG |
| | S-SrtA-His₁₀ nucleotide | GAATTCTGGGACAACCTTGAGAAAGAGACGGAAGGCCT |
| | sequence, shown in FIG. | TCGCCAGGAGATGTCAAAAGACCTTGAGGAAGTGAAGG |
| | 15A. | CTAAGGTACAACCCTATCTGGACGATTTTCAAAAGAAGT |
| | | GGCAAGAAGAAATGGAGTTGTATCGTCAAAAAGTTGAA |
| | | CCTTTGGGGGAGGAGATGCGTGATCGCGCCCGCGCCCA |
| | | CGTGGATGCATTGCGCACGCATTTAGCTCCATATAGTGA |
| | | TGAGTTGCGCCAGCGTTTGGCCGCACGTTTAGAGGCTTT |
| | | GAAAGAGAATGGCGGTGCCCGTCTGGCCGAGTACCATG |
| | | CAAAGGCGACAGAACATTTGTCCACCTTGAGCGAGAAA |
| | | GCTAAACCGGCTCTGGAGGACTTGCGTCAGGGCTTGCTT |
| | | CCGGTACTTGAATCATTCAAGGTGTCCTTTCTGTCTGCCT |
| | | TAGAAGAGTATACTAAGAAGCTTAACACACAACTGCCT |
| | | GGCACAGGTGCTGCAGCTTTAGAGGGTACCCTGGTGCCG |
| | | CGCAGCCAAGCTAAACCTCAAATTCCGAAAGATAAATC |
| | | GAAAGTGGCAGGCTATATTGAAATTCCAGATGCTGATAT |
| | | TAAAGAACCAGTATATCCAGGACCAGCAACACCTGAAC |
| | | AATTAAATAGAGGTGTAAGCTTTGCAGAAGAAAATGAA |
| | | TCACTAGATGATCAAAATATTTCAATTGCAGGACACACT |
| | | TTCATTGACCGTCCGAACTATCAATTTACAAATCTTAAA |
| | | GCAGCCAAAAAAGGTAGTATGGTGTACTTTAAAGTTGGT |
| | | AATGAAACACGTAAGTATAAAATGACAAGTATAAGAGA |
| | | TGTTAAGCCTACAGATGTAGGAGTTCTAGATGAACAAA |
| | | AAGGTAAAGATAAACAATTAACATTAATTACTTGTGATG |
| | | ATTACAATGAAAAGACAGGCGTTTGGGAAAAACGTAAA |
| | | ATCTTTGTAGCTACAGAAGTCAAACTCGAGCACCACCAC |
| | | CACCACCACCATCATCATCATTGA |
| 29 | MSP7- | ATGGCTTCGTCCGAGAATTTGTACTTCCAAGGATCGACG |
| | LPGTGAAALEGTLVPR | TTTTCCAAGTTACGCGAACAGTTAGGACCCGTAACGCAG |
| | S-SrtA-His₁₀ nucleotide | GAATTCTGGGACAACCTTGAGAAAGAGACGGAAGGCCT |
| | sequence, shown in FIG. | TCGCCAGGAGATGTCAAAAGACCTTGAGGAAGTGAAGG |
| | 15B. | CTAAGGTACAACCCTTGGGGGAGGAGATGCGTGATCGC |
| | | GCCCGCGCCCACGTGGATGCATTGCGCACGCATTTAGCT |
| | | CCATATAGTGATGAGTTGCGCCAGCGTTTGGCCGCACGT |
| | | TTAGAGGCTTTGAAAGAGAATGGCGGTGCCCGTCTGGCC |
| | | GAGTACCATGCAAAGGCGACAGAACATTTGTCCACCTTG |
| | | AGCGAGAAAGCTAAACCGGCTCTGGAGGACTTGCGTCA |
| | | GGGCTTGCTTCCGGTACTTGAATCATTCAAGGTGTCCTTT |
| | | CTGTCTGCCTTAGAAGAGTATACTAAGAAGCTTAACACA |
| | | CAACTGCCTGGCACAGGTGCTGCAGCTTTAGAGGGTACC |
| | | CTGGTGCCGCGCAGCCAAGCTAAACCTCAAATTCCGAA |
| | | AGATAAATCGAAAGTGGCAGGCTATATTGAAATTCCAG |
| | | ATGCTGATATTAAAGAACCAGTATATCCAGGACCAGCA |
| | | ACACCTGAACAATTAAATAGAGGTGTAAGCTTTGCAGA |
| | | AGAAAATGAATCACTAGATGATCAAAATATTTCAATTGC |
| | | AGGACACACTTTCATTGACCGTCCGAACTATCAATTTAC |
| | | AAATCTTAAAGCAGCCAAAAAAGGTAGTATGGTGTACT |
| | | TTAAAGTTGGTAATGAAACACGTAAGTATAAAATGACA |
| | | AGTATAAGAGATGTTAAGCCTACAGATGTAGGAGTTCTA |
| | | GATGAACAAAAAGGTAAAGATAAACAATTAACATTAAT |
| | | TACTTGTGATGATTACAATGAAAAGACAGGCGTTTGGGA |
| | | AAAACGTAAAATCTTTGTAGCTACAGAAGTCAAACTCG |
| | | AGCACCACCACCACCACCACCATCATCATCATTGA |
| 30 | MSP6- | ATGGCTTCGTCCGAGAATTTGTACTTCCAAGGATCGACG |
| | LPGTGAAALEGTLVPR | TTTTCCAAGTTACGCGAACAGTTAGGACCCGTAACGCAG |
| | S-SrtA-His₁₀ nucleotide | GAATTCTGGGACAACCTTGAGAAAGAGACGGAAGGCCT |
| | sequence, shown in FIG. | TCGCCAGGAGATGTCAAAAGACCTTGAGGAAGTGAAGG |
| | 15C. | CTAAGGTACAACCCTATAGTGATGAGTTGCGCCAGCGTT |
| | | TGGCCGCACGTTTAGAGGCTTTGAAAGAGAATGGCGGT |
| | | GCCCGTCTGGCCGAGTACCATGCAAAGGCGACAGAACA |
| | | TTTGTCCACCTTGAGCGAGAAAGCTAAACCGGCTCTGGA |
| | | GGACTTGCGTCAGGGCTTGCTTCCGGTACTTGAATCATT |
| | | CAAGGTGTCCTTTCTGTCTGCCTTAGAAGAGTATACTAA |
| | | GAAGCTTAACACACAACTGCCTGGCACAGGTGCTGCAG |
| | | CTTTAGAGGGTACCCTGGTGCCGCGCAGCCAAGCTAAA |
| | | CCTCAAATTCCGAAAGATAAATCGAAAGTGGCAGGCTA |
| | | TATTGAAATTCCAGATGCTGATATTAAAGAACCAGTATA |
| | | TCCAGGACCAGCAACACCTGAACAATTAAATAGAGGTG |
| | | TAAGCTTTGCAGAAGAAAATGAATCACTAGATGATCAA |
| | | AATATTTCAATTGCAGGACACACTTTCATTGACCGTCCG |
| | | AACTATCAATTTACAAATCTTAAAGCAGCCAAAAAAGG |
| | | TAGTATGGTGTACTTTAAAGTTGGTAATGAAACACGTAA |
| | | GTATAAAATGACAAGTATAAGAGATGTTAAGCCTACAG |
| | | ATGTAGGAGTTCTAGATGAACAAAAAGGTAAAGATAAA |
| | | CAATTAACATTAATTACTTGTGATGATTACAATGAAAAG |
| | | ACAGGCGTTTGGGAAAAACGTAAAATCTTTGTAGCTACA |
| | | GAAGTCAAACTCGAGCACCACCACCACCACCACCATCA |
| | | TCATCATTGA |
| 31 | MSP11- | ATGGCTAGCAGCGAAAACCTGTATTTTCAGGGCAGCACC |
| | LPGTGAAALEGTLVPR | TTTAGCAAACTGCGTGAACAGCTGGGCCCGGTGACCCA |
| | S-SrtA-His₁₀ nucleotide | GGAATTTTGGGATAACCTGGAAAAAGAAACCGAAGGCC |
| | sequence, shown in FIG. | TGCGTCAGGAAATGAGCAAAGATCTGGAAGAGGTGAAA |
| | 15D. | GCGAAAGTGCAGCCGTATCTGGATGACTTTCAGAAAAA |
| | | ATGGCAGGAAGAGATGGAACTGTATCGTCAGAAAGTGG |
| | | AACCGCTGCGTGCGGAACTGCAGGAAGGCGCGCGTCAG |
| | | AAACTGCATGAACTGCAGGAAAAACTGAGCCCGCTGGG |
| | | CGAAGAGATGCGTGATCGTGCGCGTGCGCATGTGGATG |
| | | CGCTGCGTACCCATCTGGCGCCGTATAGCGATGAACTGC |
| | | GTCAGCGTCTGGCGGCCCGTCTGGAAGCGCTGAAAGAA |
| | | AACGGCGGTGCGCGTCTGGCGGAATATCATGCGAAAGC |
| | | GACCGAACATCTGAGCACCCTGAGCGAAAAAGCGAAAC |
| | | CGGCGCTGGAAGATCTGCGTCAGGGCCTGCTGCCGGTG |
| | | CTGGAAAGCTTTAAAGTGAGCTTTCTGAGCGCGCTGGAA |
| | | GAGTATACCAAAAAACTGAACACCCAGCTGCCGGGTAC |
| | | GGGCGCCGCTGCACTGGAAGGTACCCTGGTGCCGCGCA |
| | | GCCAAGCTAAACCTCAAATTCCGAAAGATAAATCGAAA |
| | | GTGGCAGGCTATATTGAAATTCCAGATGCTGATATTAAA |
| | | GAACCAGTATATCCAGGACCAGCAACACCTGAACAATT |
| | | AAATAGAGGTGTAAGCTTTGCAGAAGAAAATGAATCAC |
| | | TAGATGATCAAAATATTTCAATTGCAGGACACACTTTCA |
| | | TTGACCGTCCGAACTATCAATTTACAAATCTTAAAGCAG |
| | | CCAAAAAAGGTAGTATGGTGTACTTTAAAGTTGGTAATG |
| | | AAACACGTAAGTATAAAATGACAAGTATAAGAGATGTT |
| | | AAGCCTACAGATGTAGGAGTTCTAGATGAACAAAAAGG |
| | | TAAAGATAAACAATTAACATTAATTACTTGTGATGATTA |
| | | CAATGAAAAGACAGGCGTTTGGGAAAAACGTAAAATCT |
| | | TTGTAGCTACAGAAGTCAAACTCGAGCACCACCACCAC |
| | | CACCACCATCATCATCATTGA |
| 32 | MSP20- | ATGGCCAGTTCTGAAAACCTGTATTTTCAGGGATCGACG |
| | LPGTGAAALEGTLVPR | TTTTCCAAGTTACGTGAGCAGTTAGGACCTGTTACACAA |
| | S-SrtA-His₁₀ nucleotide | GAGTTCTGGGATAACTTAGAGAAAGAGACAGAAGGGCT |
| | sequence, shown in FIG. | GCGTCAAGAGATGAGTAAAGACCTTGAAGAAGTTAAAG |
| | 15E | CAAAGGTTCAGCCCTATCTGGATGATTTCCAGAAGAAAT |
| | | GGCAGGAGGAAATGGAATTATACCGTCAGAAGGTAGAG |
| | | CCACTTCGTGCAGAATTGCAAGAAGGCGCACGCCAGAA |
| | | GTTACACGAACTGCAAGAAAAATTATCACCTTTAGGGG |
| | | AGGAGATGCGCGACCGTGCACGCGCGCACGTTGACGCC |
| | | TTACGTACGCATCTGGCGCCGTACTCTGACGAATTACGT |
| | | CAGCGCTTAGCCGCGCGCTTAGAGGCCTTAAAGGAGAA |
| | | CGGGGGAGCGCGTCTTGCAGAGTACCATGCCAAAGCCA |
| | | CGGAACATCTGTCCACCTTGAGCGAGAAGGCGAAGCCA |
| | | GCACTGGAAGACTTACGCCAGGGTTTACTGCCAGTCCTT |
| | | GAGTCTTTTAAAGTATCGTTTCTTTCTGCGCTTGAGGAAT |
| | | ACACGAAGAAGTTAAACACTCAGGGTACTCCAGTTACA |
| | | CAGGAGTTTTGGGATAATTTAGAAAAAGAGACTGAAGG |
| | | GCTTCGCCAAGAGATGTCGAAGGATTTAGAAGAGGTAA |
| | | AGGCGAAGGTCCAACCTTACCTGGACGATTTCCAGAAG |
| | | AAGTGGCAAGAAGAAATGGAGTTATACCGTCAGAAAGT |
| | | CGAACCTTTACGTGCCGAATTACAAGAAGGAGCACGCC |
| | | AAAAACTTCATGAGCTTCAGGAGAAGCTGTCCCCCCTTG |
| | | GTGAAGAGATGCGCGACCGTGCGCGTGCTCATGTAGAT |
| | | GCATTACGTACCCACCTTGCCCCCTATAGCGATGAGTTA |
| | | CGTCAGCGTCTTGCCGCCCGCCTGGAAGCTTTAAAAGAG |
| | | AATGGCGGTGCTCGTTTAGCAGAGTATCACGCCAAGGCC |
| | | ACCGAACATCTTTCAACTTTATCTGAGAAAGCCAAACCT |
| | | GCGTTAGAAGACTTACGTCAAGGGCTTCTGCCTGTCTTA |
| | | GAGTCGTTCAAGGTTTCATTTCTGTCGGCGCTTGAAGAA |
| | | TATACTAAAAAGTTAAATACACAGTTACCTGGTACAGGT |
| | | GCTGCAGCTTTAGAGGGTACCCTGGTGCCGCGCAGCCA |
| | | AGCTAAACCTCAAATTCCGAAAGATAAATCGAAAGTGG |
| | | CAGGCTATATTGAAATTCCAGATGCTGATATTAAAGAAC |
| | | CAGTATATCCAGGACCAGCAACACCTGAACAATTAAAT |
| | | AGAGGTGTAAGCTTTGCAGAAGAAAATGAATCACTAGA |
| | | TGATCAAAATATTTCAATTGCAGGACACACTTTCATTGA |
| | | CCGTCCGAACTATCAATTTACAAATCTTAAAGCAGCCAA |
| | | AAAAGGTAGTATGGTGTACTTTAAAGTTGGTAATGAAAC |
| | | ACGTAAGTATAAAATGACAAGTATAAGAGATGTTAAGC |
| | | CTACAGATGTAGGAGTTCTAGATGAACAAAAAGGTAAA |
| | | GATAAACAATTAACATTAATTACTTGTGATGATTACAAT |
| | | GAAAAGACAGGCGTTTGGGAAAAACGTAAAATCTTTGT |
| | | AGCTACAGAAGTCAAACTCGAGCACCACCACCACCACC |
| | | ACCATCATCATCATTGA |
| 33 | G-SFTI- | ATGGCCAGTTCTTTACCTCGTGACGCGGAAAACCTGTAT |
| | LPGT(GGS)5LVPRS- | TTTCAGGGACGCTGCACCAAAAGCATTCCGCCGATTTGC |
| | SrtA-His₁₀ nucleotide | TTTCCGGATCTGCCTGGTACCGGGGGATCGGGAGGTTCA |
| | sequence, shown in FIG. | GGTGGGTCCGGTGGTAGTGGTGGGAGTCTCGTGCCGCGC |
| | 16A. | TCCCAAGCTAAACCTCAAATTCCGAAAGATAAATCGAA |
| | | AGTGGCAGGCTATATTGAAATTCCAGATGCTGATATTAA |
| | | AGAACCAGTATATCCAGGACCAGCAACACCTGAACAAT |
| | | TAAATAGAGGTGTAAGCTTTGCAGAAGAAAATGAATCA |
| | | CTAGATGATCAAAATATTTCAATTGCAGGACACACTTTC |
| | | ATTGACCGTCCGAACTATCAATTTACAAATCTTAAAGCA |
| | | GCCAAAAAAGGTAGTATGGTGTACTTTAAAGTTGGTAAT |
| | | GAAACACGTAAGTATAAAATGACAAGTATAAGAGATGT |
| | | TAAGCCTACAGATGTAGGAGTTCTAGATGAACAAAAAG |
| | | GTAAAGATAAACAATTAACATTAATTACTTGTGATGATT |
| | | ACAATGAAAAGACAGGCGTTTGGGAAAAACGTAAAATC |
| | | TTTGTAGCTACAGAAGTCAAACTCGAGCACCACCACCAC |
| | | CACCACCATCATCATCATTGA |
| 34 | G-kB1- | ATGGCCAGTTCTTTACCTCGTGACGCGGAAAACCTGTAT |
| | LPGT(GGS)5LVPRS- | TTTCAGGGATGCGGCGAAACCTGCGTGGGCGGCACCTG |
| | SrtA-His₁₀ nucleotide | CAACACCCCGGGCTGCACCTGCAGCTGGCCGGTGTGCA |
| | sequence, shown in FIG. | CCCGCAACGGCCTGCCGGTGACCGGGGGATCGGGAGGT |
| | 16B. | TCAGGTGGGTCCGGTGGTAGTGGTGGGAGTCTCGTGCCG |
| | | CGCTCCCAAGCTAAACCTCAAATTCCGAAAGATAAATC |
| | | GAAAGTGGCAGGCTATATTGAAATTCCAGATGCTGATAT |
| | | TAAAGAACCAGTATATCCAGGACCAGCAACACCTGAAC |
| | | AATTAAATAGAGGTGTAAGCTTTGCAGAAGAAAATGAA |
| | | TCACTAGATGATCAAAATATTTCAATTGCAGGACACACT |
| | | TTCATTGACCGTCCGAACTATCAATTTACAAATCTTAAA |
| | | GCAGCCAAAAAAGGTAGTATGGTGTACTTTAAAGTTGGT |
| | | AATGAAACACGTAAGTATAAAATGACAAGTATAAGAGA |
| | | TGTTAAGCCTACAGATGTAGGAGTTCTAGATGAACAAA |
| | | AAGGTAAAGATAAACAATTAACATTAATTACTTGTGATG |
| | | ATTACAATGAAAAGACAGGCGTTTGGGAAAAACGTAAA |
| | | ATCTTTGTAGCTACAGAAGTCAAACTCGAGCACCACCAC |
| | | CACCACCACCATCATCATCATTGA |
| 35 | Amino acid linker L1, | GAAALEGTQAKP |
| | shown in FIG. 2. | |
| 36 | Amino acid linker L1a, | GGSGGSGGQAKP |
| | shown in FIG. 2. | |
| 37 | Amino acid linker L1b, | GAAALEGTLVPRS |
| | shown in FIG. 2. | |
| 38 | Amino acid linker L2, | GGSGGSGGSGGSGGS |
| | shown in FIG. 2. | |
| 39 | Amino acid linker L2b, | GGSGGSGGSGGSGGSLVPRS |
| | shown in FIG. 2. | |
| 40 | G-SFTI-LPGT amino acid | GRCTKSIPPICFPDLPGT |
| | sequence, shown in FIG. | |
| | 4. | |
| 41 | G-(kB1-LPV)T amino | GCGETCVGGTCNTPGCTCSWPVCTRNGLPVT |
| | acid sequence, shown in | |
| | FIG. 4. | |
| 42 | GG-Vc1.1-LPGT amino | GGCCSDPRCNYDHPEICGLPGT |
| | acid sequence, shown in | |
| | FIG. 4. | |
| 43 | Optimized nucleotide | TCTAGAAATAATTTTGTTTAACTTTAAGAAGGAGATATA |
| | sequence for an mRNA | CATATGGCCAGTTCTTTACCTCGTGACGCGGAAAACCTG |
| | translation initiation | TATTTTCAG |
| | region 1, shown in FIG. | |
| | 12. | |
| 44 | Optimized nucleotide | TCTAGAAATAATTTTGTTTAACTTTAAGAAGGAGATATA |
| | sequence for an mRNA | CATATGGCCAGTTCTCTACCCCGTGATGCGGAAAACCTG |
| | translation initiation | TATTTTCAG |
| | region 2, shown in FIG. | |
| | 12. | |
| 45 | Optimized nucleotide | TCTAGAAATAATTTTGTTTAACTTTAAGAAGGAGATATA |
| | sequence for an mRNA | CATATGGCTAGTTCCCTACCCCGTGATGCAGAGAATCTG |
| | translation initiation | TACTTTCAG |
| | region 3, shown in FIG. | |
| | 12. | |
| 46 | Optimized nucleotide | TCTAGAAATAATTTTGTTTAACTTTAAGAAGGAGATATA |
| | sequence for an mRNA | CATATGGCTTCCTCCCTTCCACGCGACGCAGAGAATTTG |
| | translation initiation | TATTTCCAG |
| | region 4, shown in FIG. | |
| | 12. | |
| 47 | Optimized nucleotide | TCTAGAAATAATTTTGTTTAACTTTAAGAAGGAGATATA |
| | sequence for an mRNA | CATATGGCAAGTTCACTCCCTCGGGACGCAGAAAATCTG |
| | translation initiation | TACTTTCAA |
| | region 5, shown in FIG. | |
| | 12. | |
| 48 | Optimized nucleotide | TCTAGAAATAATTTTGTTTAACTTTAAGAAGGAGATATA |
| | sequence for an mRNA | CATATGGCCAGTTCGTTGCCCCGTGATGCTGAGAATCTG |
| | translation initiation | TACTTCCAA |
| | region 6, shown in FIG. | |
| | 12. | |
| 49 | Optimized nucleotide | TCTAGAAATAATTTTGTTTAACTTTAAGAAGGAGATATA |
| | sequence for an mRNA | CATATGGCTTCGAGTTTACCACGTGACGCTGAGAATCTG |
| | translation initiation | TACTTCCAG |
| | region 7, shown in FIG. | |
| | 12. | |
| 50 | Optimized nucleotide | TCTAGAAATAATTTTGTTTAACTTTAAGAAGGAGATATA |
| | sequence for an mRNA | CATATGGCCTCGTCTTTACCCCGTGATGCAGAGAATCTG |
| | translation initiation | TATTTTCAA |
| | region 8, shown in FIG. | |
| | 12. | |
| 51 | Optimized nucleotide | TCTAGAAATAATTTTGTTTAACTTTAAGAAGGAGATATA |
| | sequence for an mRNA | CATATGGCCTCTTCCCTTCCGCGCGATGCAGAGAACTTG |
| | translation initiation | TATTTCCAA |
| | region 9, shown in FIG. | |
| | 12. | |
| 52 | P1-forward | CGGGAATCCGGTACCCAAGCTAAACCTCAAATTCCGAA |
| | oligonucleotide, for wt- | AG |
| | SrtA. | |
| 53 | P1-reverse | TTTTTTCCGCTCGAGTTTGACTTCTGTAGCTACAAAGATT |
| | oligonucleotide. | TTACG |
| 54 | P2-forward | TATACATATGGCTTCGTCCCACCATCAC |
| | oligonucleotide, for | |
| | Gly2Ala. | |
| 55 | P2-reverse | TCTCCTTCTTAAAGTTAAACAAAATTATTTC |
| | oligonucleotide. | |
| 56 | P3-forward | CGCGGATCCCATATGGCTAGCAGCGAAAACCTGTATTTT |
| | oligonucleotide, for | CAGGGCAGCACC |
| | MSP11. | |
| 57 | P3-reverse | GGCGAATTCGGTACCCGGCAGCTGGGTG |
| | oligonucleotide. | |
| 58 | P4-forward | GAGAATTTGTACTTCCAAGGATC |
| | oligonucleotide, for | |
| | removing N-terminal His- | |
| | tag. | |
| 59 | P4-reverse | GGACGAAGCCATATGTATATC |
| | oligonucleotide. | |
| 60 | P5-forward | CATCATTGAGATCCGGCTGCTAAC |
| | oligonucleotide, for | |
| | introducing His₄ into His₆ | |
| | at the c-terminal. | |
| 61 | P5-reverse | ATGATGGTGGTGGTGGTGGTGGTG |
| | oligonucleotide. | |
| 62 | P6-forward | TGGGTCCGGTGGTAGTGGTGGGAGTCAAGCTAAACCGC |
| | oligonucleotide, for | AGATC |
| | introducing (GGS)x5 | |
| | linker in MSP9-eSrtA. | |
| 63 | P6-reverse | CCTGAACCTCCCGATCCCCCGGTACCAGGCAGTTGTGTG |
| | oligonucleotide. | TTAAG |
| 64 | P7-forward | TGGGTCCGGTGGTAGTGGTGGGAGTCAAGCTAAACCTC |
| | oligonucleotide, for | AAATTC |
| | introducing (GGS)x5 | |
| | linker in MSP9-wtSrtA. | |
| 65 | P7-reverse | CCTGAACCTCCCGATCCCCCGGTACCAGGCAGTTGTGTG |
| | oligonucleotide = P6- | TTAAG |
| | reverse oligonucleotide. | |
| 66 | P8-forward | TTGGGGGAGGAGATGCGT |
| | oligonucleotide, for | |
| | deleting H4 in MSP9 to | |
| | make MSP7. | |
| 67 | P8-reverse | GGGTTGTACCTTAGCCTTCAC |
| | oligonucleotide. | |
| 68 | P9-forward | TATAGTGATGAGTTGCGC |
| | oligonucleotide, for | |
| | deleting H4 and H₆ in | |
| | MSP9 to make MSP6. | |
| 69 | P9-reverse | GGGTTGTACCTTAGCCTTC |
| | oligonucleotide. | |
| 70 | P10-forward | CGATGCCGAGAATTTGTACTTCCAAGG |
| | oligonucleotide, for | |
| | introducing an inhibitor | |
| | peptide. | |
| 71 | P10-reverse | CGTGGCAAGGACGAAGCCATATGTATATC |
| | oligonucleotide. | |
| 72 | P11-forward | CGCGGAAAACCTGTATTTTCAGGGATCGACGTTTTCCAA |
| | oligonucleotide, | G |
| | optimized inhibitory | |
| | peptide, option1. | |
| 73 | P11-reverse | TCACGAGGTAAAGAACTGGCCATATGTATATCTCCTTCT |
| | oligonucleotide. | TAAAGTTAAAC |
| 74 | P12-forward | CGCAGAGAATTTGTATTTCCAGGGATCGACGTTTTCCAA |
| | oligonucleotide, | G |
| | optimized inhibitory | |
| | peptide, option2. | |
| 75 | P12-reverse | TCGCGTGGAAGGGAGGAAGCCATATGTATATCTCCTTCT |
| | oligonucleotide. | TAAAGTTAAAC |
| 76 | P13-forward | GCGCAGCCAAGCTAAACCTCAAATTCC |
| | oligonucleotide, for | |
| | introducing a Thrombin | |
| | site between | |
| | LPGTGAAALEGT linker | |
| | and SrtA. | |
| 77 | P13-reverse | GGCACCAGGGTACCCTCTAAAGCTGC |
| | oligonucleotide. | |
| 78 | SEQ ID NO: 78 - P14- | GCGCTCCCAAGCTAAACCTCAAATTCCGAAAG |
| | forward oligonucleotide, | |
| | for introducing a | |
| | Thrombin site between | |
| | LPGTG(GGS)5 linker and | |
| | SrtA. | |
| 79 | P14-reverse | GGCACGAGACTCCCACCACTACCACC |
| | oligonucleotide. | |
| 80 | P15-forward | GAAAACCTGTATTTTCAGGG |
| | oligonucleotide, for | |
| | removing N-terminal his | |
| | tag in MSP11- | |
| | LPGTGAAALEGTLVPR | |
| | S-SrtA-His₁₀. | |
| 81 | P15-reverse | GCTGCTAGCCATATGTATATC |
| | oligonucleotide. | |
| 82 | P16-forward | AAGAAGGAGATATACATATGGCCAGTTCTGAAAACCTGT |
| | oligonucleotide, for | ATTTTCAGGGATCGACG |
| | amplification of MSP20 | |
| | and replace MSP9 in | |
| | MSP9- | |
| | LPGTGAAALEGTLVPR | |
| | S-wtSrtA-His₁₀. | |
| 83 | P16-reverse | CACCAGGGTACCCTCTAAAGCTGCAGCACCTGTACCAG |
| | oligonucleotide. | GTAACTGTGTATTTAACTTTTTAGTATATTCTTC |
| 84 | P17-forward | CTGCCTGGTACCGGGGGA |
| | oligonucleotide, for | |
| | generating empty | |
| | autocyclase-L2a vector. | |
| 85 | P17-reverse | TCCCTGAAAATACAGGTTTTCCGCG |
| | oligonucleotide. | |
| 86 | P18-forward | GCCGATTTGCTTTCCGGATCTGCCTGGTACCGGGGGA |
| | oligonucleotide, to | |
| | generate autocyclase-L2a- | |
| | G-SFTI. | |
| 87 | P18-reverse | GGAATGCTTTTGGTGCAGCGTCCCTGAAAATACAGGTTT |
| | oligonucleotide. | TCCGCG |
| 88 | P19-forward | CACCTGCAGCTGGCCGGTGTGCACCCGCAACGGCCTGCC |
| | oligonucleotide, to | GGTGACCGGGGGATCGGGAGGT |
| | generate autocyclase-L2a- | |
| | G-KalataB1. | |
| 89 | P19-reverse | CAGCCCGGGGTGTTGCAGGTGCCGCCCACGCAGGTTTCG |
| | oligonucleotide. | CCGCATCCCTGAAAATACAGGTTTTCCGCG |
| 90 | P20-forward | GCCGATTTGCTTTCCGGATCTGCCTGGCACAGGTGCT |
| | oligonucleotide, to | |
| | generate autocyclase-L1b- | |
| | G-SFTI. | |
| 91 | P20-reverse | GGAATGCTTTTGGTGCAGCGTCCTTGGAAGTACAAATTC |
| | oligonucleotide. | TCGGAC |
| 92 | P21-forward, to generate | TCCGCCGATTTGCTTTCCGGATCTGCCTGGCACAGGTGC |
| | autocyclase-L1b-GGG- | T |
| | SFTI. | |
| 93 | P21-reverse | ATGCTTTTGGTGCAGCGGCCACCTCCTTGGAAGTACAAAT |
| | oligonucleotide. | TCTCGGAC |
| 94 | P22-forward | TGGGTCCGGTGGTAGTGGTGGGAGTCTGGTGCCGCGCA |
| | oligonucleotide, to | GCCAA |
| | generate autocyclase-L2a- | |
| | GGG-SFTI. | |
| 95 | P22-reverse | CCTGAACCTCCCGATCCCCCGGTACCAGGCAGATCCGGAA |
| | oligonucleotide. | AGCAAATCGG |
| 96 | P23-forward | GGTGGCTGCGGCGAAACCTGCGTG |
| | oligonucleotide, to | |
| | generate autocyclase-L1b- | |
| | GGG-kB1. | |
| 97 | P23-reverse | TCCTTGGAAGTACAAATTCTCGGACG |
| | oligonucleotide | |
| 98 | P24-forward | GTCCGGTGGTAGTGGTGGGAGTCTGGTGCCGCGCAGCC |
| | oligonucleotide, to | AA |
| | generate autocyclase-L2a- | |
| | GGG-kB1. | |
| 99 | P24-reverse | CCACCTGAACCTCCCGATCCCCCTGTCACAGGCAGGCCG |
| | oligonucleotide. | TTG |
| 100 | P25-forward | CTATGATCATCCGGAAATTTGCGGTCTGCCTGGCACAGG |
| | oligonucleotide, to | TGCT |
| | generate autocyclase-L1b- | |
| | GG-Vc1.1. | |
| 101 | P25-reverse | TTGCAGCGCGGATCGCTGCAGCAACCTCCTTGGAAGTAC |
| | oligonucleotide. | AAATTCTCGGAC |
| 102 | P26-forward | TGGGTCCGGTGGTAGTGGTGGGAGTCTGGTGCCGCGCA |
| | oligonucleotide, to | GCCAA |
| | generate autocyclase-L2a- | |
| | GG-Vc1.1. | |
| 103 | P26-reverse | CCTGAACCTCCCGATCCCCCGGTACCAGGCAGACCGCA |
| | oligonucleotide. | AATTTCCGG |
| 104 | Sortase A (SrtA) amino | LPXTG/A |
| | acid recognition motif, | [LPXTGA] |
| | where X is any amino acid | |
| | 1. | |
| 105 | Sortase A (SrtA) amino | LAXTG |
| | acid recognition motif, | |
| | where X is any amino acid | |
| | 2. | |
| 106 | Sortase A (SrtA) amino | LPXSG |
| | acid recognition motif, | |
| | where X is any amino acid | |
| | 3. | |
| 107 | Sortase A (SrtA) amino | LPGTG/A |
| | acid recognition motif 1. | [LPGTGA] |
| 108 | Sortase A (SrtA) amino | LPSTG/A |
| | acid recognition motif 2. | [LPSTGA] |
| 109 | Sortase A (SrtA) amino | LPETG/A |
| | acid recognition motif 3. | [LPETGA] |
| 110 | Sortase A (SrtA) amino | LPGTG |
| | acid recognition motif 4. | |
| 111 | Sortase A (SrtA) amino | LPGTA |
| | acid recognition motif 5. | |
| 112 | Sortase A (SrtA) amino | LPSTG |
| | acid recognition motif 6. | |
| 113 | Sortase A (SrtA) amino | LPSTA |
| | acid recognition motif 7. | |
| 114 | Sortase A (SrtA) amino | LPETG |
| | acid recognition motif 8. | |
| 115 | Sortase A (SrtA) amino | LPETA |
| | acid recognition motif 9. | |
| 116 | Sortase A (SrtA) amino | LPATG |
| | acid recognition motif 10. | |
| 117 | eSrtA(4S-9) amino acid | LPXSG |
| | recognition motif, where | |
| | X is any amino acid. | |
| 118 | Sortase A (SrtA) amino | LPXTG |
| | acid recognition motif, | |
| | where X is any amino acid | |
| | 4. | |
| 119 | eSrtA(2A-9) amino acid | LAXTG |
| | recognition motif, where | |
| | X is any amino acid. | |
| 120 | SrtA-F40 and SrtA-A1-22 | APXTG |
| | amino acid recognition | |
| | motif, where X is any | |
| | amino acid. | |
| 121 | SrtA-F1-20 amino acid | FPXTG |
| | recognition motif, where | |
| | X is any amino acid. | |
| 122 | SrtAβ amino acid | LMVGG |
| | recognition motif. | |
| 123 | Amino acid linker/spacer, | GS(GGS)_{N} |
| | where n is an integer of at | |
| | least one, two, three, four, | |
| | or five. | |
| 124 | Amino acid linker/spacer | GAAA |
| | 1. | |
| 125 | Amino acid linker/spacer | LEGT |
| | 2. | |
| 126 | Amino acid spacer/linker | GAAALEGT |
| | 3. | |
| 127 | Amino acid spacer/linker | GS(GGS)4 |
| | 4. | [GS GGS GGS GGS GGS] |
| 128 | Amino acid spacer/linker | LPGTGAAALEGT |
| | 5. | |
| 129 | Amino acid spacer/linker | LPGT(GGS)5 |
| | 6. | [LPGT GGS GGS GGS GGS GGS] |
| 130 | Amino acid sequence of | LVPRS |
| | Thrombin cleavage site. | |
| 131 | Amino acid spacer/linker | GGSGGSGG |
| | 7. | |
| 132 | Amino acid sequence of | LPRDA |
| | inhibitory peptide before | |
| | TEV recognition site. | |
| 133 | Amino acid sequence of | GLU-ASN-LEU-TYR-PHE-GLN-(GLY/SER) |
| | TEV protease cleavage | [ENLYFQGS] |
| | site 1. | |
| 134 | Amino acid sequence of | ENLYFQG |
| | TEV protease cleavage | |
| | site 2. | |
| 135 | Optimized amino acid | GAAALEGT |
| | spacer/linker 1. | |
| 136 | Optimized amino acid | GS(GGS)4 |
| | spacer/linker 2. | [GS GGS GGS GGS GGS] |
| 137 | Polyhistidine (affinity) tag | HHHHHHHHHH |
| | 1. | |
| 138 | Polyhistidine (affinity) tag | HHHHHH |
| | 2. | |
| 139 | Amino acid sequence of | QAKP |
| | flexible segment of srtA. | |
| 140 | 5′-UTR Sequence, shown | TCTAGAAATAATTTTGTTTAACTTTAAGAAGGAGATATA |
| | in FIG. 12. | CAT |
| 141 | Protein coding sequence, | ATGGCTTCGTCCTTGCCACGCGATGCCGAGAATTTGTAC |
| | shown in FIG. 12. | TTCCAA |
| 142 | 16s rRNA sequence, | AAGAAGGAGA |
| | shown in FIG. 12. | |
| 143 | KpnI nucleotide | GGTACC |
| | recognition site. | |
| 144 | Butelase-1 amino acid | ASX-HIS-VAL |
| | recognition motif, where | |
| | Asx is Asn (N) or Asp (D). | |
| 145 | Butelase-1 amino acid | ASP-HIS-VAL |
| | recognition motif. | |
| 146 | OaAEP1 amino acid | ASN-GLY-LEU |
| | recognition motif. | |
| 147 | AEP amino acid | X₁X₂X₃ |
| | recognition motif, where | |
| | X₁ is N or D; X₂ is G or S; | |
| | and X₃ is L, A or I. | |
| 148 | AEP amino acid acceptor | X₄X₅ |
| | motif, wherein X₄ is | |
| | optional and any amino | |
| | acid or G, Q, K, V or L; | |
| | and X₅ is optional or any | |
| | amino acid or L, F or I or a | |
| | hydrophobic amino acid | |
| | residue. | |
| 149 | Amino acid linker/spacer | (G)ₙ |
| | polymer, where n is an | |
| | integer of at least one, two, | |
| | three, four, or five. | |
| 150 | Amino acid linker/spacer | (G₁₋₅S₁₋₅)ₙ |
| | polymer, where n is an | |
| | integer of at least one, two, | |
| | three, four, or five. | |
| 151 | Amino acid linker/spacer. | GGS |

### EXAMPLE

### Introduction

The present Example was designed to overcome the shortcomings of the conventional enzyme catalyzed cyclisation of proteins. Below is described the first example of a unimolecular cyclisation reaction, which has a fundamentally different reaction mechanism and behaves according to first order reaction kinetics over a wide range of concentrations. The reaction is performed by a new family of proteins we have termed ‘autocyclases’, where the ligase is fused to the protein being cyclised. We present a workflow for use of autocyclases for production of cyclic proteins (including peptides) which includes expression, purification and cyclisation. The general utility of autocyclases is demonstrated by circularisation of two challenging systems: (1) α-helical membrane scaffold proteins (MSPs) for making circular nanodiscs (cNDs) and (2) disulfide-rich cyclotides.

Methods

### Reagents and Plasmids

All lipids were purchased from Avanti Polar Lipids, Inc. (Alabaster, AL) or Sigma-Aldrich. Enzymes and buffers used for polymerase chain reaction and molecular cloning were purchased from Genesearch, the exclusive Australian distributor of New England Biolabs (NEB) molecular biology products. The Quick-stick ligase was purchased from Bioline. The DNA miniprep kit was bought from Qiagene. The gel extraction and PCR clean-up kit were ordered from Macherey-Nagel. All sequencing was performed by Sanger sequencing at the Australian Genome Research Facility (AGRF). All primers and codon-optimized gene fragments were ordered from Integrated DNA technology (IDT).

The plasmids for expressing evolved P94R/D160N/D165A/K190E/K196T sortase A and MSP11 were kindly provided by Prof David Liu and Prof. Gerhard Wagner respectively, both at Harvard University.

### The Molecular Cloning of Autocyclase for MSPs

The amino acid sequences of the MSPs were based on literature reports (including MSP9 and MSP11²², MSP6 and MSP7⁴³, and MSP20⁴¹). A codon-optimized gene fragment was designed to encode N-terminal His₆, TEV site, MSP9 and evolved sortase A (eSrtA) (His₆-G2-MSP9-LPGTGAAALEGT-eSrtA-His₆). To facilitate the replacement of SrtA gene, a KpnI site (GGTACC) was introduced before the SrtA gene. This fusion gene was ordered from IDT, digested with NdeI/XhoI to generate an overhang for cloning into the pET29 vector and cleaned using a PCR clean up kit. The eSrtA expression plasmid was digested with NdeI/XhoI³⁰ and purified by DNA agarose electrophoresis to generate the pET29a vector. The vector was then ligated with a suitable gene fragment (via NdeI/XhoI sites) to deliver a vector expressing N-terminal His₆, TEV site, MSP9 and evolved sortase A (eSrtA).

To generate the fusion protein of MSP9-LPGTGAAALEGT-wild type SrtA (His₆-G2-MSP9-LPGTGAAALEGT-wtSrtA-His₆), SrtA-staph-A59 was amplified using suitable primers (P1 pairs—Table 1). The PCR product was digested with KpnI and XhoI to replace the eSrtA fragment of His₆-G2-MSP9-LPGTGAAALEGT-eSrtA-His₆ to yield His₆-G2-MSP9-LPGTGAAALEGT-wtSrtA-His₆. The mutation of Gly2 to Ala2 in both MSP9-wild type SrtA and pentamutant SrtA was achieved using the primer P2 pairs (Table 1) with NEB Q5 mutagenesis kit and home-made CaCl₂) competent cells.

The removal of N-terminal His₆-tag in His₆-MSP9-LPGTGAAALEGT-wtSrtA/eSrtA-His₆ was achieved using the primer P4 pairs while the introduction of four more histidines into His₆-tag at the C-terminus was achieved using the P5 primer pairs (Table 1), yielding MSP9-LPGTGAAALEGT-wtSrtA/eSrtA-His₁₀.

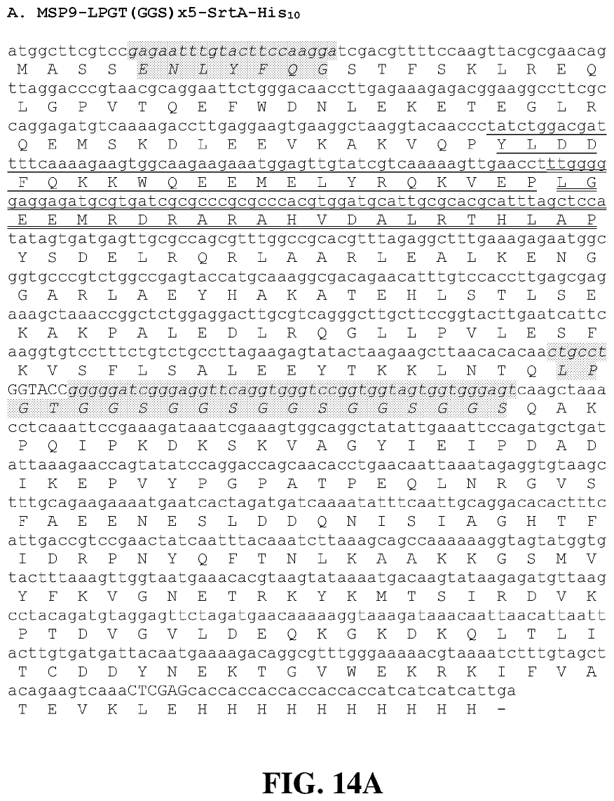

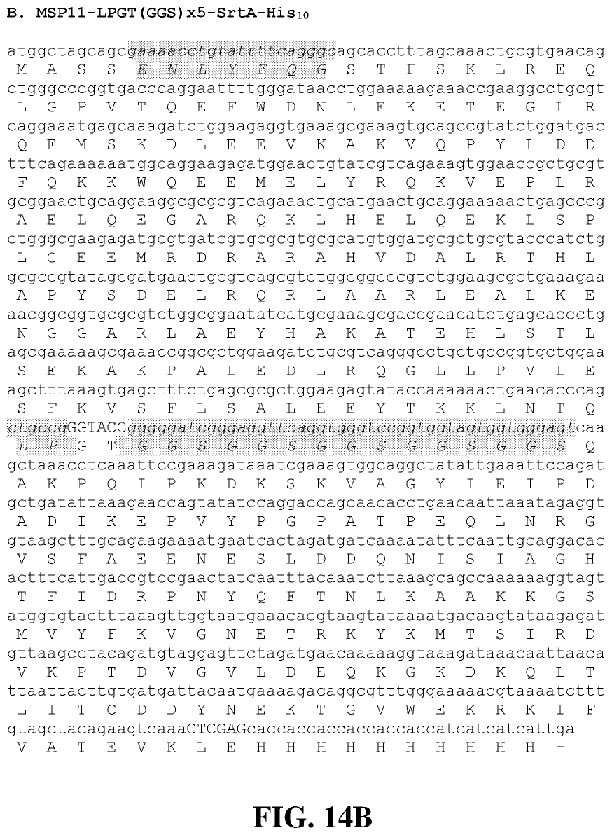

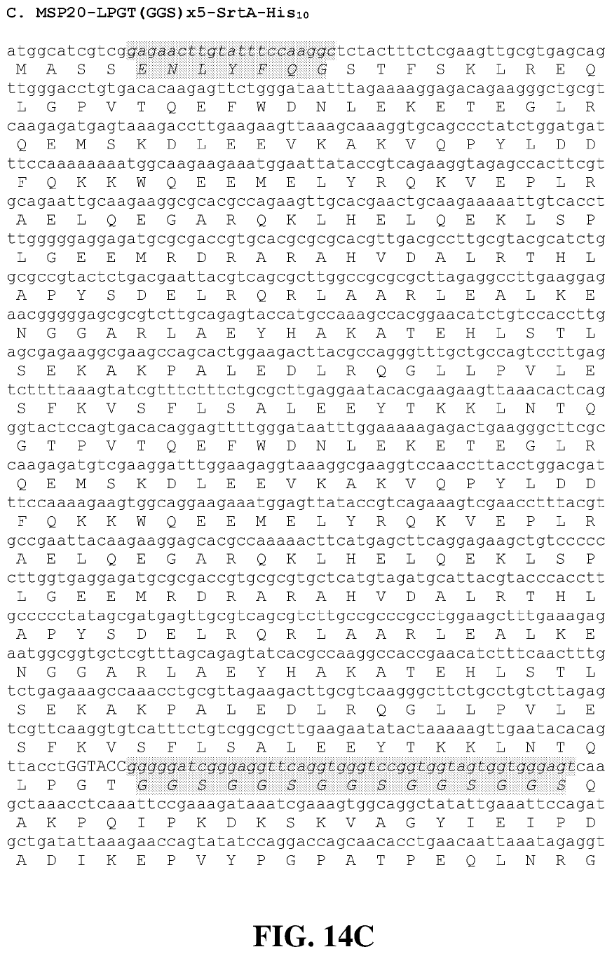

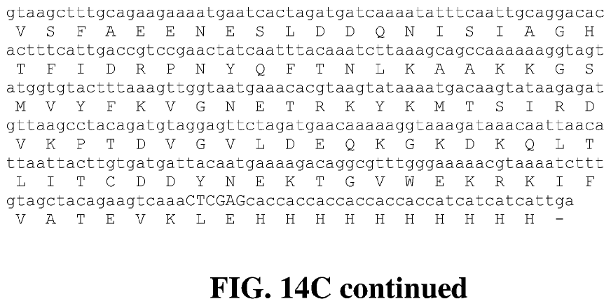

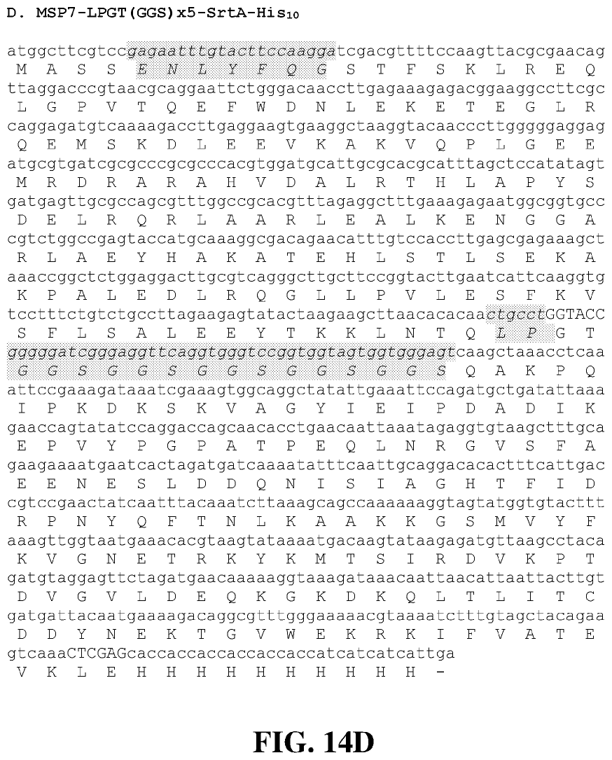

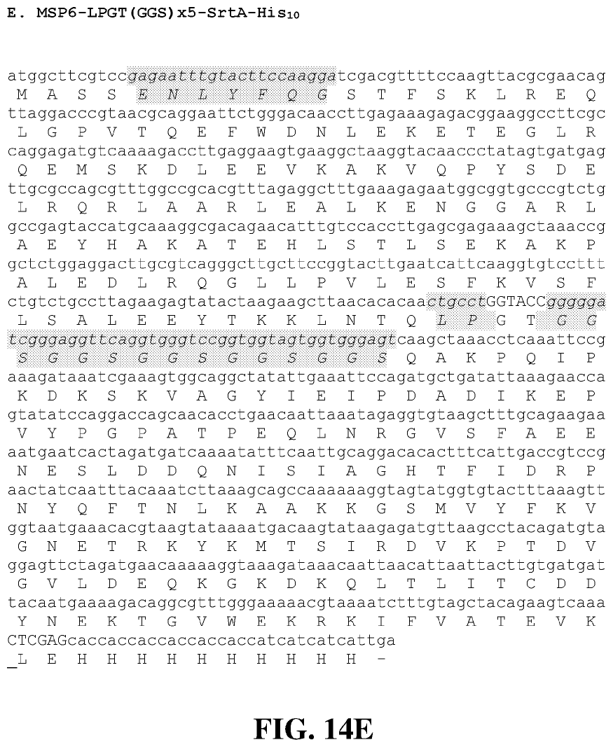

The linker replacement in MSP9-LPGTGAAALEGT-wtSrtA-His₁₀ and MSP9-LPGTGAAALEGT-eSrtA-His₁₀ was achieved using the P6 and P7 primer pairs respectively (Table 1), yielding MSP9-LPGT(GGS)5-wtSrtA-His₁₀ (FIG. 14 (A)) or MSP9-LPGT(GGS)5-eSrtA-His₁₀.

To generate the fusion of MSP11—LPGT(GGS)5-wtSrtA-His₁₀, MSP11 was amplified from MSP1D1²² expression plasmid using the primer P3 pair. The PCR product was digested with NdeI/KpnI to replace the MSP9 fragment of MSP9—LPGT(GGS)5-wtSrtA-His₁₀ and to yield MSP11—LPGT(GGS)5-wtSrtA-His₁₀ (FIG. 14 (B)).

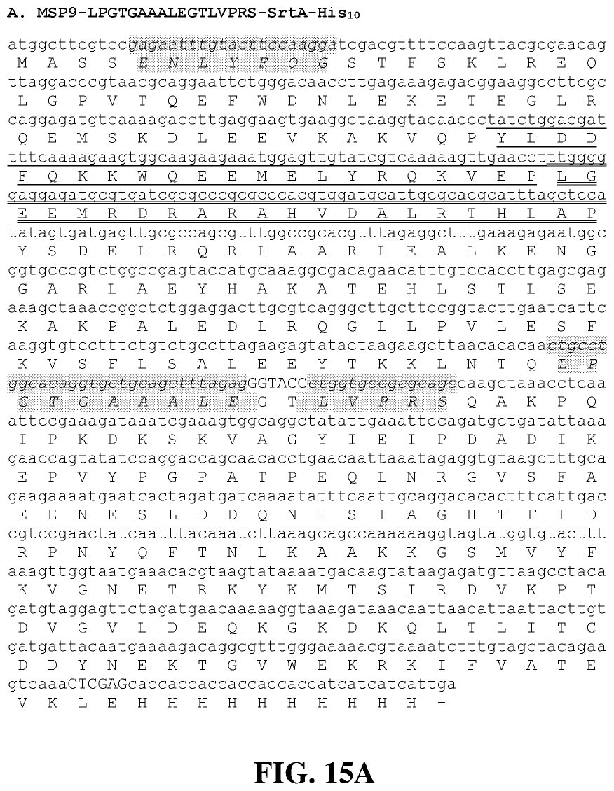

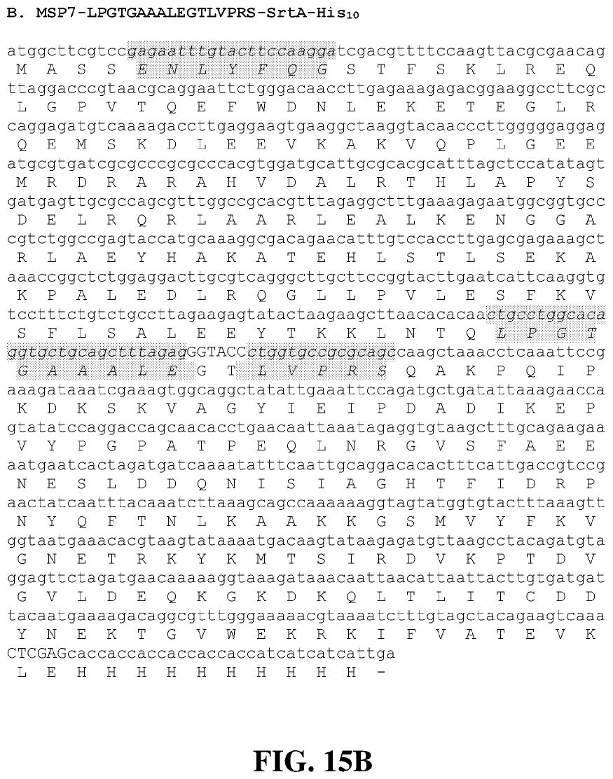

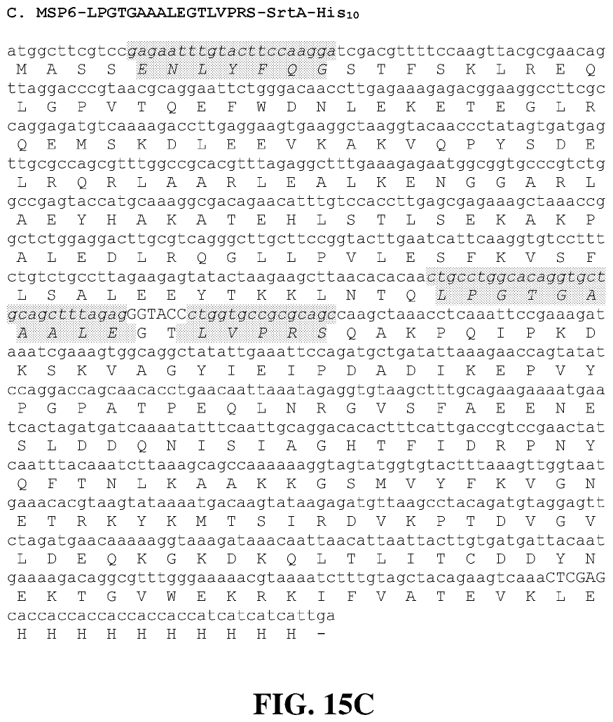

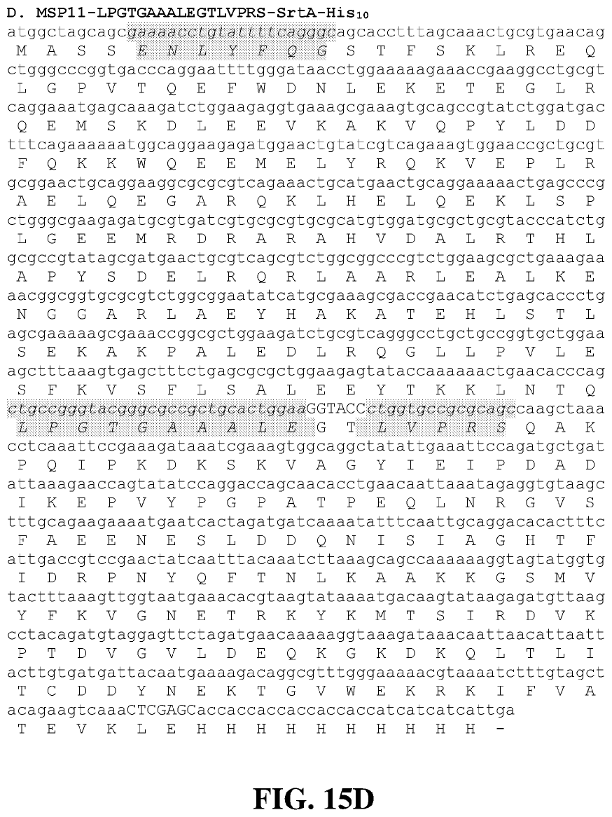

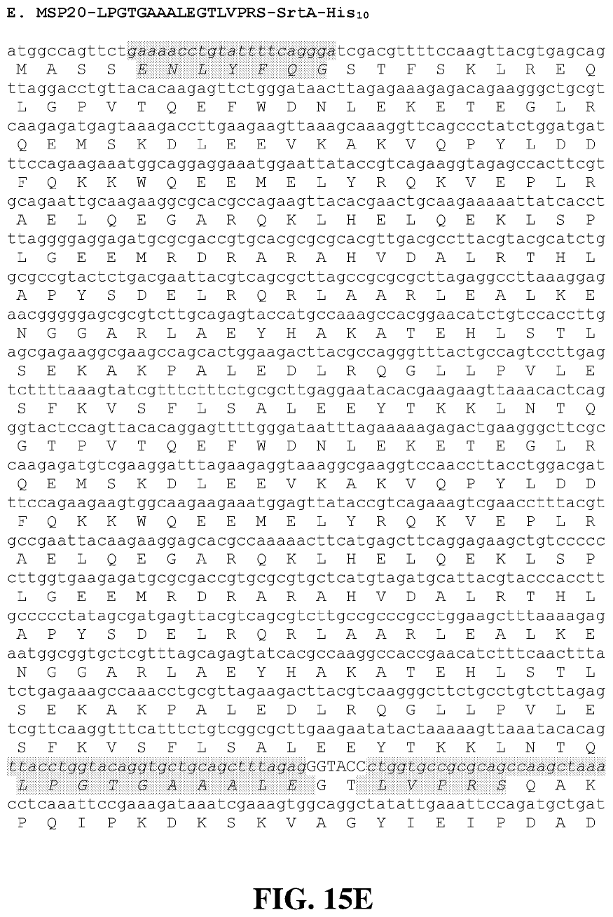

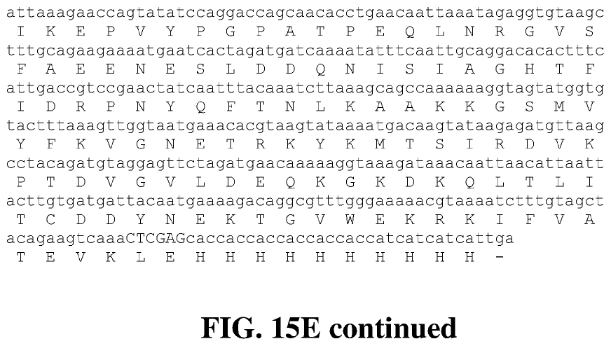

To generate the fusion of MSP20-linker-wtSrtA, codon optimized MSP20 gene block was digested with NdeI/KpnI to replace the MSP9 fragment at MSP9—LPGT(GGS)5-wtSrtA-His₁₀ to yield MSP20—LPGT(GGS)5-wtSrtA-His₁₀ (FIG. 14(C)). Primer pair P8 was used to delete H4 in MSP9—LPGT(GGS)5-wtSrtA-His₁₀ to generate MSP7—LPGT(GGS)5-wtSrtA-His₁₀ (FIG. 14(D)). Primer pair P9 was used to delete H4 and H6 in MSP9—LPGT(GGS)5-wtSrtA-His₁₀ to generate MSP6—LPGT(GGS)5-wtSrtA-His₁₀ (FIG. 14(E)). Primer pair P10 was used to introduce a SrtA inhibitory peptide for the sake of in vivo inhibition of SrtA activity, which led to disabling of expression—the optimized pair P12 resulted in resumed expression. Primer pair P13 was used to introduce a thrombin site between LPGTGAAALEGT linker and SrtA while P14 (between LPGT(GGS)5 linker and SrtA) was used for analyzing the extra bands during intramolecular circularisation of the fusion protein and recycling of SrtA. The Primer pair P13 was used to generate MSP9-LPGTGAAALEGTLVPRS-wtSrtA-His₁₀ (FIG. 15(A)), and pair P8 to generate MSP7-LPGTGAAALEGTLVPRS-wtSrtA-His₁₀ (FIG. 15(B)) and pair P9 to generate MSP6-LPGTGAAALEGTLVPRS-wtSrtA-His₁₀ (FIG. 15(C)). For producing MSP11-LPGTGAAALEGTLVPRS-wtSrtA-His₁₀ (FIG. 15(D)), MSP11 was cut out from a plasmid containing the MSP11 gene with NdeI/KpnI, and was subsequently ligated into the vector of MSP9-LPGTGAAALEGTLVPRS-wtSrtA-His₁₀ from which MSP9 was removed and finally the N-terminal his-tag of MSP11 was removed by the primer pair P15. For constructing MSP20-LPGTGAAALEGTLVPRS-wtSrtA-His₁₀ (FIG. 15(E)), the MSP20 gene containing plasmid pUCIDT-AMP+ vector was purchased from IDT. Primer pair P16 was then used to amplify MSP20, digested with NdeI/KpnI and replaced MSP9 in MSP9-LPGTGAAALEGTLVPRS-wtSrtA-His₁₀ (FIG. 15(A)).

### The Molecular Cloning of Autocyclases for Cyclotides

We used the primer pair P17 to delete MSP9 in A2-inhibitory-peptide-TEV-MSP9-LPGT(GGS)5LVPRS-SrtA-His₁₀ to generate the empty autocyclase vector A2-inhibitory-peptide-TEV-LPGT(GGS)5LVPRS-SrtA-His₁₀ (autocyclase-L2a vector). In this autocyclase-L2a vector, primer pair P18 was used to insert G-SFTI between TEV site and linker L2a, and primer pair P19 was used to insert G-kB1. To generate SFTI in autocyclase-L1b, primer pair P20 was used to insert G-SFTI and P21 for GGG-SFTI between TEV site and linker L1b. Then GGG-SFTI in autocyclase-L2a was made by replacing L1b with L2a linker using primer pair P22.

To generate G-kB1 in autocyclase-L1b, a gene block coding G-kB1 was ordered from IDT and replaced MSP9 in autocyclase-L1b-MSP9 to generate autocyclase-L1b-G-kB1. Autocyclase-L1b-G-kB1 was converted to Autocyclase-L1b-GGG-kB1 by the primer pair P23. Subsequently autocyclase-L1b-GGG-kB1 was changed into autocyclase-L2a-GGG-kB1 by the primer pair P24.

Autocyclase-L1b-GG-Vc1.1 was constructed by replacing MSP9 in autocyclase-L1b-MSP9 by GG-Vc1.1 with the primer pair P25. Primer pair P26 was used in PCR mutagenesis to replace L1b by L2a to produce autocyclase-L2a-GG-Vc1.1 (Table 1).

### SDS-Page Analysis

Protein samples were run on 12 or 15% SDS-page gels (made in-house) under electrophoresis at 180-200 V for 30-50 mins. The Precision Plus Protein Dual Xtra ladder (Bio-Rad) was used as a detectable standard, consisting of 20, 25, 37, 50, 75, 100, 150 and 250 kDa markers. After electrophoresis, gels were washed with warm distilled water three times for 3-5 minutes for each wash, prior to staining with Coomassie Brilliant Blue (CBB), then destained in distilled water⁵⁰. Band intensity of Coomassie staining was quantified using ImageLab software (Bio-Rad). Circularisation efficiency was calculated as the intensity of circular protein bands divided by the intensity of the intact autocyclase.

### Autocyclase Expression and Purification

Each fusion protein expression construct (in pET29 vector) was transformed into E. coli BL21(DE3) cells. The freshly transformed colonies or a glycerol stock was inoculated into 10 mL LB media containing 50 μg/mL of kanamycin. LB media was then shaken at 30° C. at 220 rpm overnight for a preculture. 1% of the preculture (3 mL) was used to inoculate 300 mL LB broth containing 50 μg/mL of kanamycin. The culture was then incubated at 37° C. (shaking at 250 rpm) until the OD₆₀₀ reached about 1.0. Induction was commenced by the addition of 0.2 mM IPTG and the culture was left shaking at 250 rpm for 1-6 h at 30° C. The cells were harvested by centrifugation in 0.5 L bottles using a JLA-10.500 rotor (Beckman) operating at 6,000 g for 10 min at 4° C. and the cell pellets were stored at −20° C. The cell pellets were resuspended in lysis buffer (25 mM sodium phosphate, 500 mM NaCl, pH 7.4, 20 mM imidazole) plus 1 mg/mL lysozyme and were stirred at 4° C. for 0.5 hour. The resuspended cells were lysed by sonication (digital sonifier 450 Branson) on ice (40% power, 3 s on and 12 s off) for 5 min and the suspension was mixed for a better cooling, followed by repeated sonication.

The sonicated sample was centrifuged in 50 mL bottles using a JA-25.50 rotor (Beckman) at 30,000 g for 30 min at 4° C. The supernatant was loaded onto a gravity column containing 3 mL Ni-NTA resin (pre-equilibrated with 4° C. lysis buffer). The column was washed with five column volumes of lysis buffer. The autocyclase was then eluted with five column volumes of elution buffer (25 mM sodium phosphate, 500 mM NaCl, pH 7.4, 500 mM imidazole).

The collected fractions from the column were supplemented with 20 mM EDTA and buffer exchanged into the reaction buffer (20 mM Tris HCl pH 7.5, 150 mM NaCl) using 10,000 MW cutoff Amicon centrifugal filter unit through centrifugation (Sigma 2-16K Centrifuge, SciQuip) at 4000 g, 4° C. The protein solution was then supplemented with 1 mM β-mercapto ethanol (BME), 0.5 mM EDTA and TEV at TEV to protein ratio of 1 mg: 50 mg for TEV protease cleavage overnight at 4° C. The cleaved protein sample was then spun down at 3000 g for 5 min at 4° C. to remove any precipitates.

### MSP and Cyclotides Circularization

For MSP cyclisation, TEV protease-cleaved autocyclase was kept at a total protein concentration<100 μM in equilibration buffer (20 mM Tris HCl pH 7.5, 150 mM NaCl, 0.5 mM EDTA, 1 mM β-mercapto ethanol). The sample was supplemented with 1 mM DDM and 10 mM CaCl₂) to initiate cyclisation. The reaction was carried out at 37° C. for 6-8 hours or overnight with shaking at 200 rpm.

For cyclotide cyclisation, the autocyclase was also cleaved by TEV protease to expose N-terminal glycine. The reaction was initialized at a total concentration of <100 μM in the reaction buffer (20 mM Tris HCl pH 7.5, 150 mM NaCl, 10 mM CaCl₂)). The reaction was supplemented with 3 mM GSH/0.3 mM GSSG to produce cyclised and oxidized cyclotides. The glutathione can be replaced by 3 mM β-mercaptoethanol for producing cyclised and reduced peptides.

cMSPs Purification

Following completion of the reaction, the reaction mixture was filtered or centrifuged to remove any precipitation and directly loaded onto a gravity column containing 3 mL Ni-NTA resin (pre-equilibrated with the reaction buffer, i.e. 20 mM Tris HCl pH 7.5, 150 mM NaCl). The flow-through containing cyclised MSP products was collected while generated free SrtA remained on the column.

cMSPs were buffer exchanged into equilibration buffer (20 mM Tris HCl pH 7.5, 1 mM DDM) using 10,000 MW cutoff Amicon centrifugal filter unit through centrifugation (4,000 g, 4° C.). The sample was loaded onto a 5 mL HiScreen Q HP (GE Healthcare) anion-exchange column at 4° C. and purified by an AKTA FPLC system (GE Healthcare). A flow rate was 0.3 mL/min and a linear gradient of 0 to 25% of equilibration buffer supplemented with 1 M NaCl over 20 column volumes was applied. Chromatograms were recorded as A₂₈₀ over volume (mL) and samples were fractionated as 4 mL fractions through an automated fraction collector (Frac-920) module (GE Healthcare). The fractions containing>95% pure cMSPs as judged by SDS-page were pooled together, concentrated to about 0.5 mM, aliquoted and flash frozen to be stored at −80° C. or used directly for nanodisc assembly.

### Sortase a Regeneration

After MSP cyclisation, the unreacted autocyclase, if any, SrtA with the linker and SrtA with MSP degradants in the case of MSP and other by-products were captured on Ni-NTA resin. The resin was rinsed with a buffer containing 20 mM Tris HCl pH 7.5, 50 mM NaCl and incubated with thrombin protease (sigma T4648-1KU) at a 1 unit: 1 mg ratio of thrombin protease: fusion protein at room temperature for six hours or overnight. After thrombin cleavage, the resin was washed with the same buffer and the SrtA eluted with the elution buffer (25 mM sodium phosphate, 500 mM NaCl, 500 mM imidazole, pH 7.4). Finally, the collected eluant for pure SrtA was buffer exchanged into 20 mM Tris·HCl, 100 mM NaCl, pH 7.5, 2 mM DTT by a 10,000 Mw Amicon Centricon (Merck Millipore) through centrifugation with a Refrigerated Sigma 2-16K Centrifuge (SciQuip) at 4000 rcf, 4° C. Aliquots of the SrtA were flash frozen and stored at −80° C. Wild-type sortase A (wtSrtA) concentration was calculated from the measured A₂₈₀ using the extinction coefficient of 17,420 M⁻¹ cm⁻¹ (https://web.expasy.org/protparam/).

### ND Production

Lipid stocks, stored at −80° C., of powder masses of either POPC, POPG and DOTAP (all Tm<4° C.) were suspended in reconstitution buffer (25 mM Tris pH 7.5, 100 mM NaCl, 0.5 mM EDTA and 100 mM cholate) and always handled on ice.

To assemble cNWs, [lipid]/[cMSP] ratios were defined based on past literature, using the equation: N_{L}×S=(0.423×M−9.75)², where N_{L} is the number of lipids per ND, M is the number of amino acids in the scaffold protein and S is the mean surface area per lipid used to form the lipid-nanodisc, measured in Å^{2 58}. POPC and POPG have been estimated to have a similar mean surface area of around 70 Å^{2 59}. We therefore determined ratios of x:1, x:1, 40:1, 50:1 and 60:1 for cMSP6, cMSP7, cMSP9, cMSP11 and cMSP20, respectively. MSP and lipid were both dissolved at the desired ratio and rocked for 1 hour at 4° C. Subsequently, 0.6 g of Bio-Beads SM-2 per mL of solution were added to absorb detergent and initiate ND assembly. The mixture was gently stirred for 4 h at 4° C. The solution was filtered through a 0.45 μm PES membrane to remove the Bio-Beads and then concentrated using an Amicon 10 kDa Centricon at 4° C. and 3000 g. The assembled discs were injected into size exclusion chromatography to monitor aggregation behavior in the buffer of 20 mM Tris·HCl, 50 mM NaCl, 1 mM EDTA, pH 7.5.

### Results

### The Rational Design of Autocyclases

The design of an autocyclase involves engineering several modules: (i) an activation site which is liberated by application of a suitable protease; (ii) a target protein (which could be a peptide or polypeptide) to be cyclised; (iii) a ligation recognition site; (iv) a spacer of suitable length and flexibility; (v) the ligase enzyme sequence and finally (vi) a purification/affinity tag to remove the ligase byproduct once the reaction has taken place. The ligase once liberated may contain a reactive N-terminal amino acid (glycine in the case of SrtA), making it of little value as a ligase enzyme in other applications. However, the ligase can be recovered in a useful form if an additional module is introduced between modules (iv) and (v). In our design this module (iv′) is a protease site—orthogonal to that used in step (i)—which removes the reactive N-terminal sequence of the liberated ligase. Thus, the principles of the autocyclase approach are general where each module may be optimized or swapped for module with similar properties.

The activation site (i): The N-terminus of the protein to be cyclised generally requires a specific recognition sequence. However, it is typically important that this sequence is shielded from exposure prematurely and therefore, the N-terminus of the protein is extended beyond this sequence by addition of a protease recognition sequence. Here we have inserted a TEV-protease recognition site N-terminal to the ligase recognition site. Once the autocyclase is purified the reactive N-terminal sequence may be liberated by application of TEV protease. In this case this leaves an N-terminal glycine as the substrate for the sortase A ligation reaction.

The target (ii) and recognition site (iii): The target protein (or peptide) to be cyclised generally requires N- and C-termini that are in close proximity when the protein is folded. Thus, this includes both naturally cyclic (e.g., cyclotides) and naturally linear (e.g., MSP) targets. Furthermore, the C-terminus requires a ligase recognition site for fusion to the reactive N-terminal sequence. Here, we have used two cyclotides and five MSPs as the targets and C-terminally added the SrtA recognition site to these sequences.

The linker (iv): In our design the linker fuses the SrtA recognition site LPXTG (where X denotes any amino acid) to the N-terminal end of SrtA. Thus, a linker is required that would put the LPXTG site in contact with the ligase catalytic site (i.e. catalytic C184 in FIG. 2, pdb id 2kid)²³—this includes breaching the α1/β2 and β3/β4 loops of sortase A. However, the NMR structure of SrtA (d59) reveals that the first R strand of the enzyme starts at residue 74 and that the first 14 residues do not form a well-ordered secondary structure, nor are these residues required for catalytic activity (or calcium binding). Furthermore, several class A and class C sortase enzymes contain a flexible, N-terminal segment, and structural studies have indicated that this segment can fold back over the active site of the enzyme, thereby (auto)inhibiting the enzyme while it is not required²⁴. These studies suggest that N-terminal fusions of SrtA are not only feasible, but may further benefit from the evolution of a regulatory process that directs the N-terminal region towards the catalytic site.

The structure of SrtA in complex with its substrate (LPXTG), reveals that the distance from the glycine in the LPXTG motif to the N-terminal Q64 in SrtA is ˜35 Å along the surface of the protein, corresponding to approximately a 12 amino acid linker. After subtracting the flexible N-terminal part (QAKP) of SrtA, eight amino acids would represent the shortest possible shortest linker without the need to further unfold the N-terminal region beyond this point. Here, the composition of those eight amino acids is chosen as GAAALEGT (L1 in FIG. 2), which originates from the direct fusion of MSP used for bimolecular cyclisation using the SrtA enzyme²². Furthermore, we designed a thrombin site (LVPRS)²⁵, which was substituted for the native QAKP sequence in SrtA (L1b in FIG. 2). This allows us to modify the N-terminal region of the cleaved SrtA upon completion of the autocyclase reaction, to leave only Ser in the cleaved SrtA. This strategy allows us to recycle SrtA as a valuable side product of this reaction, which can be used in other applications.

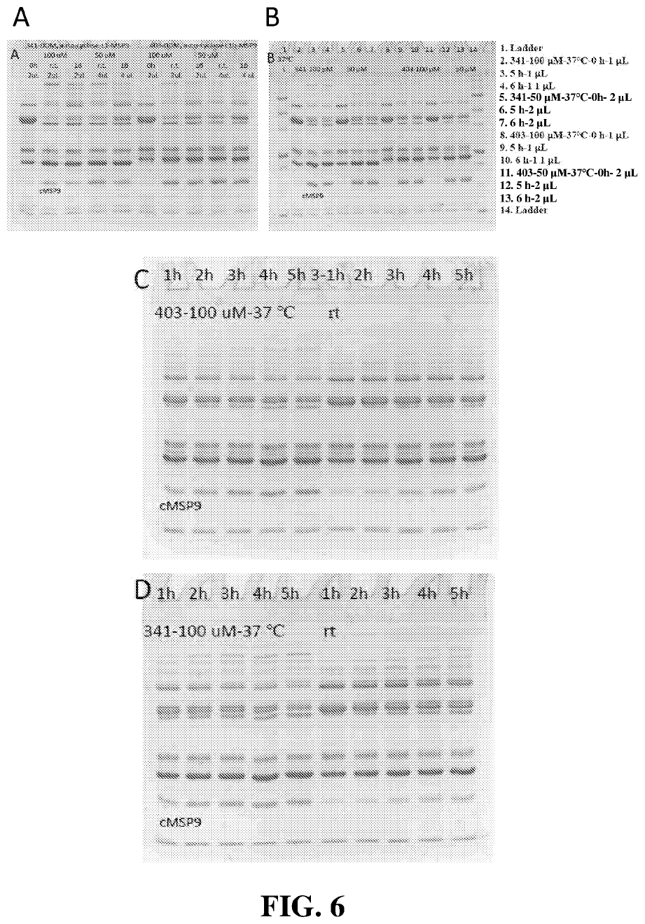

The above design was used to autocircularize an MSP9-L1-SrtA (9 refers to the diameter, in nanometers, of the nanodisc produced by this protein) and an MSP9-L1b-SrtA construct to produce cMSP9 at 4° C., 16° C., 23° C. (room temperature) and 37° C. All reactions proceeded successfully with the highest yield achieved at 37° C. (FIG. 6). We found that MSP9-L1b-SrtA produces higher quantities of cMSP9 than MSP9-L1-SrtA at all tested temperatures while the yield difference is minimal at 37° C. (FIG. 2C). The differences in yield depending on reaction temperature and linker length and composition indicate that these are important factors in dictating the unimolecular reaction rate.

To relax the conformational constraints imposed by the shorter L1 and L1b linkers, a longer linker (L2) was also engineered that included five GGS repeats (GGS)5²⁶. This is a significantly longer and more flexible linker that can easily span the distance between the active and recognition sites. Indeed, the production of cMSP9 from MSP9-L2-SrtA (FIG. 2D), is complete in just 1 hour at >23° C. Similar to the L1 linker we produced an L2b version with a thrombin cleavage site in order to remove the (GGS)5 linker and recycle SrtA upon completion of the autocyclisation reaction (FIG. 2B).

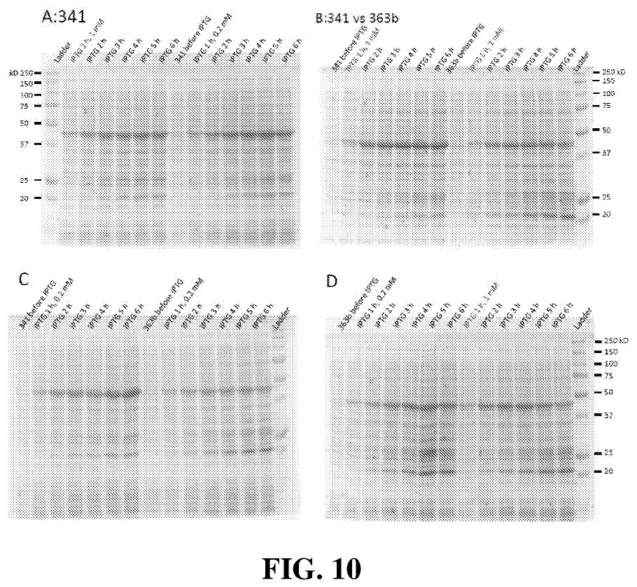

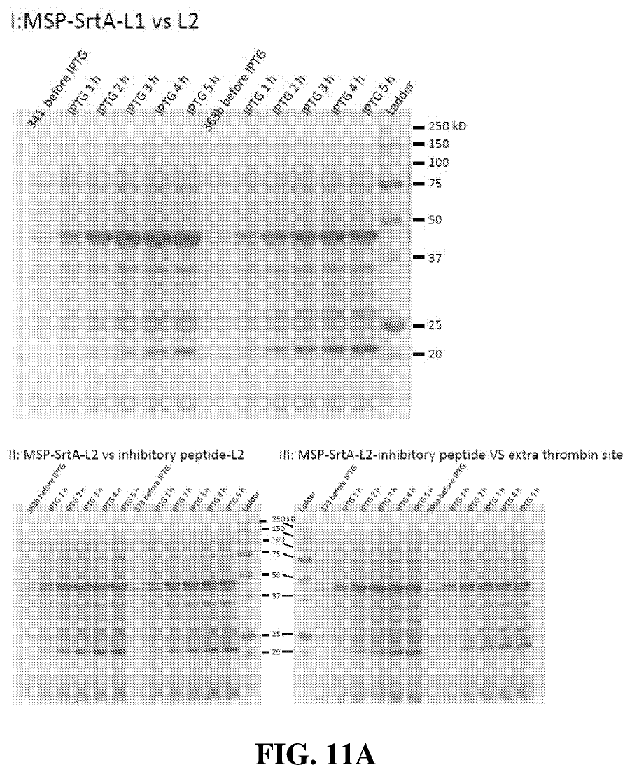

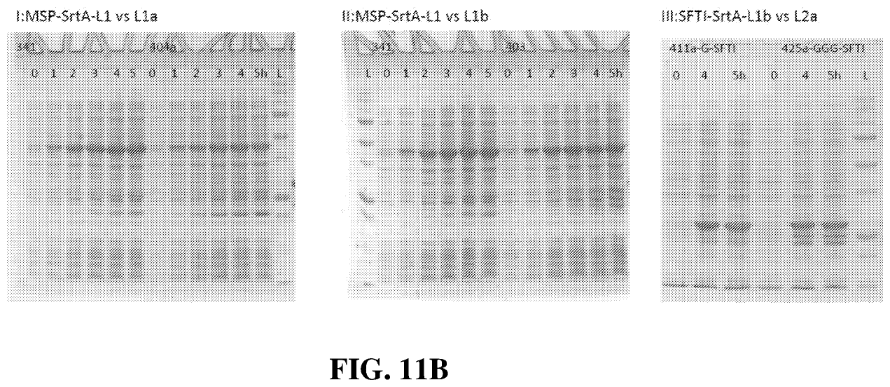

While the use of MSP9-L2-SrtA led to improved reaction kinetics, it unfortunately also led to more in vivo hydrolysis and decrease overall yields of the fusion protein (FIG. 10A). This indicated that in vivo hydrolysis of MSP containing autocyclases is sensitive to the flexibility of the linker. We further tested this by design of a short GGSGGSGG (L1a linker) to yield the autocyclase MSP9-L1a-SrtA and found that also this construct yielded less fusion protein compared to the other constructs (FIG. 10). To decrease the in vivo hydrolysis, we introduced an inhibitory peptide (LPRDA) before TEV recognition site (FIG. 11)²⁷. However, we found that the inhibitory peptide negligibly inhibited in vivo hydrolysis (FIG. 10), likely due to the low affinity of the peptide inhibitor compared to that of the recognition sequence.

The ligase: While natural cyclases are now available, SrtA remains one of the most widely utilised ligases for biochemical reactions. In addition, sortase fusions have been utilized to simplify protein purification and labeling^{26, 28, 29}.

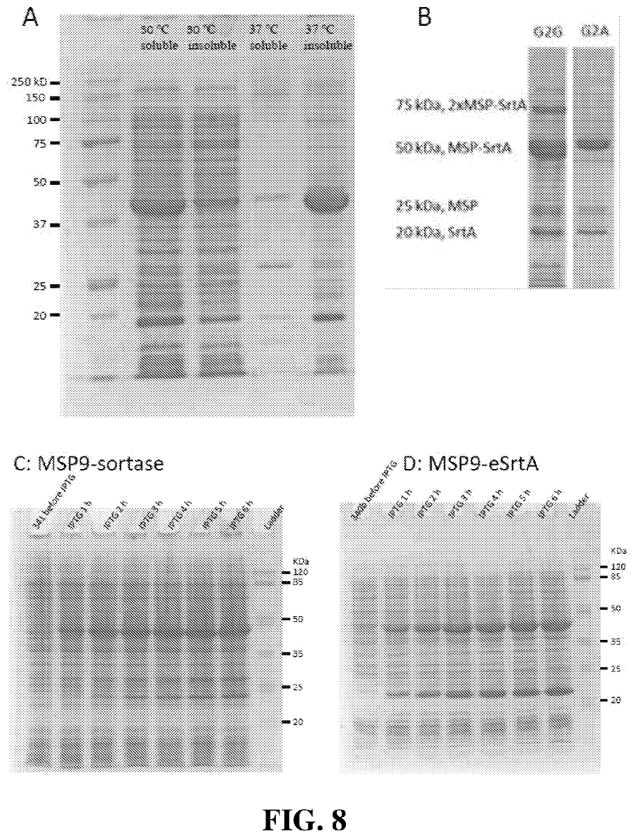

Our initial autocyclase design was constructed using the evolved sortase pentamutant (eSrt)³⁰ to prepare circular target protein²². However, we found that the fusion protein is not very soluble when expressed at 37° C., while soluble expression was found at 30° C. (FIG. 8). We also observed the presence of higher molecular weight products when using this construct (FIG. 8).

These species likely represent polymeric products. The major species present was a 75 kDa protein which corresponds to the weight of 2x(MSP-SrtA). For these polymeric products to form, an N-terminal glycine is required, and it is possible²⁸ that in E. coli an endogenous methionine aminopeptidase removes Met1 to expose Gly2³¹, which then is a substrate for the enzyme. This hypothesis was then supported by a G2A mutation that resulted in expression without the in vivo polymerization products (FIG. 8).

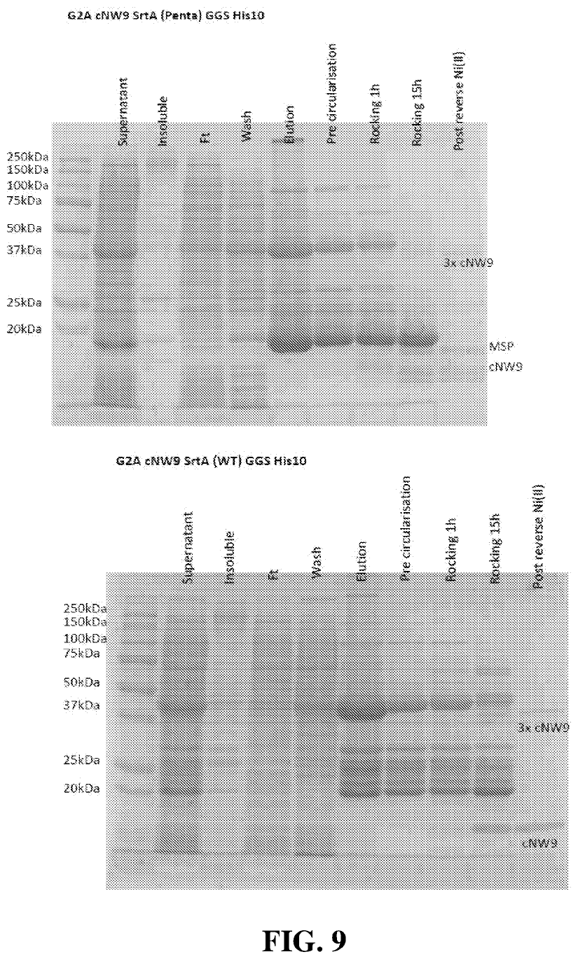

Nevertheless, even when expressing the G2A MSP9-eSrtA, we observed that ˜35% of the fusion protein underwent in vivo hydrolysis (cleavage) after three hours resulting in high levels of impurities (FIG. 9), most likely due to the enhanced activity of the evolved SrtA³⁰. This is remarkable considering the low free Ca²⁺ concentration in E. coli cells (90±10 nM)³². Consequently, eSrtA was replaced by wild-type Sortase A (SrtA), which has lower enzymatic activity (FIG. 9), thereby reducing the presence of in vivo hydrolysis to ˜10%.

The purification tag: The autocyclase can be purified after expression by use of a suitable purification tag. Here we have investigated the use of hexa- and deca-histidine tags (His₆ and His₁₀ respectively) at both termini. We find that if a hexahistidine-tag is placed at both ends of autocyclase, e.g., His₆-MSP9-SrtA-His₆, the in vivo hydrolysis will produce his-tagged linear His₆-MSP9, which downstream contaminates the product of the autocyclase reaction (data not shown).

The purification tag is therefore ideally placed, only at the C-terminal end of the autocyclase. We further found that, compared to a His₆-tag, a His₁₀-tag yielded improved purity of the final product without otherwise affecting the process.

In vivo stability of the target (iii): It is well-known that human derived MSP is prone to strong bacterial proteolysis. Faas et al. performed detailed experiments to determine the time-course and degradation rate of MSP1D1 in a standard pET expression system. Their work revealed that the maximum MSP1D1 yields are achieved at 4 h post-induction before the degradation rate overtakes the production rate³³. We performed similar time-course experiments which show that our MSP containing autocyclase also had the highest expression level of the fusion protein at 4 h post-induction at 30° C. using 1 mM IPTG for induction. Furthermore, the IPTG concentration used for SrtA fusions has previously been optimized to 0.2 mM^{28, 29}. We therefore also performed time-course experiments at 0.2 mM IPTG and 1 mM IPTG. We found that when using the longer and more flexible L2 linker protein expression peaks at 4 h regardless of IPTG concentration, the shorter more rigid L1 linker reaches a maximum after 4 hours at the higher IPTG concentration and peaks after 5-6 hours when the lower concentration is used (FIG. 8). Thus, we conclude that in vivo stability will depend on the target as well as the linker used.

### The General Application of Autocyclase

Next, we investigated the generality of the autocyclase design outlined above using different sizes of MSPs as well as a number of cyclic peptides.

The MSP-autocyclases: First, we investigated the production of cMSP6, 7, 11 and 20, by introducing these sequences into the autocyclase-L1b construct. In all cases we were able to successfully produce circularised MSPs through the procedure outlined above for cMSP9, albeit with some minor adjustments in each case.

A significant challenge in the production of MSPs is the need to reduce self-association of the protein to prevent polymerization during the cyclisation reaction. Indeed, the crystal structure³⁴ of MSP reveals that the protein forms stable dimers, which may explain the challenges in producing monomeric circular proteins. To address this issue, studies have shown that the addition of detergents (DDM³⁵ or Triton X-100³⁶) significantly reduces the presence of polymeric byproducts. Our result for the autocyclase construct is consistent with these reports and we find that the addition of 1 mM DDM dramatically improves the yield of cMSP9 (termed detergent-assisted cyclisation). Thus, we include 1 mM DDM in autocyclase reaction for producing all MSPs investigated here.

We note here that since Triton X-100 is present in the lysis buffer it will co-purify with the protein unless specific measures are taken to reduce its concentration. Indeed, we find that while addition of DDM to the MSP9-autocyclase containing co-purified Triton X-100 results in improved yields, complete removal of Triton X-100 prior to addition of DDM results in significant levels of polymeric products. Thus, our results suggest that Triton X-100 is important in the detergent-assisted cyclisation process while DDM may also be used if Triton X-100 is co-purified with the protein from earlier steps. We also note that the challenges in removal of Triton X-100 while fortuitous in the cyclisation process, does interfere with absorbance readings at 280 nm, and should be removed prior to quantitation—this can be readily verified by 1D ¹H NMR after ion exchange chromatography or treatment with biobeads.

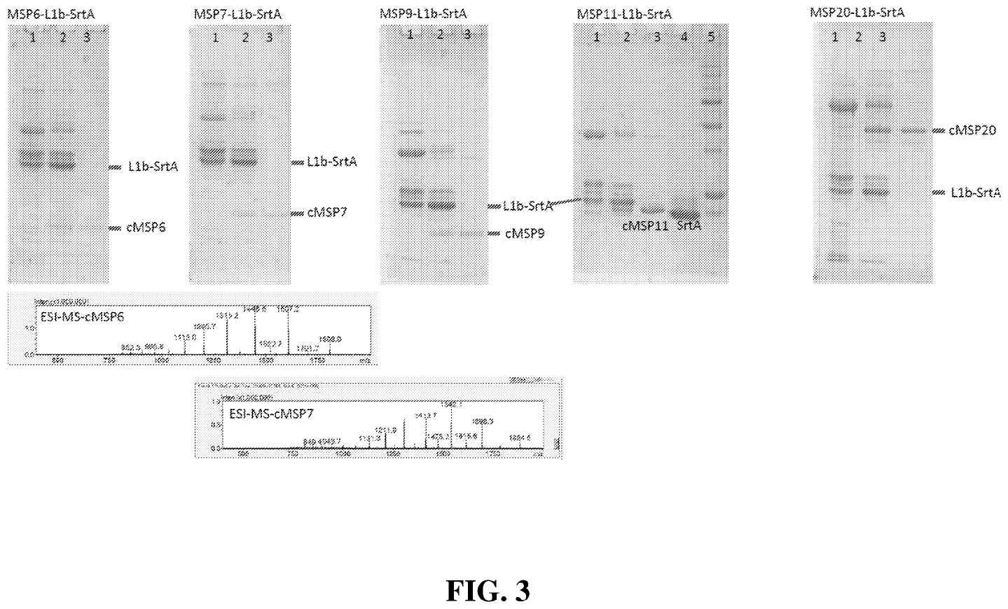

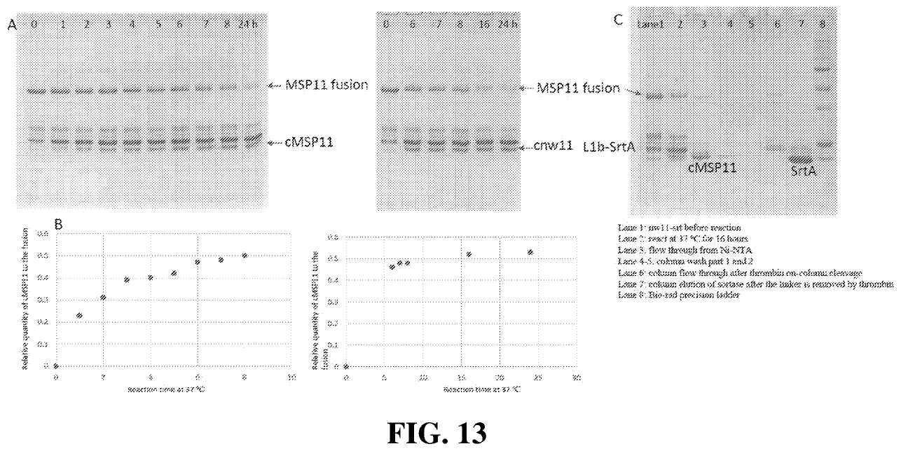

The requirement of detergents in the circularization of MSPs is also dependent on the size of the MSP. The circularization of MSP9 to cMSP9 heavily relies on the presence of 1 mM DDM while the cyclisation of cMSP20 and cMSP6 is independent of the presence of additional detergents. MSPs 6, 7, and 11 all show moderate improvements in yields (˜5%, ˜15% and ˜10% respectively) upon addition of detergents (1 mM DDM) during cyclisation. The extent of polymerization is dependent on the rate of aggregation and this of course is also influence by the protein concentration. Details of the reaction mechanism are discussed in the next section, but we note here that cMSP9 is efficiently produced at concentrations up to ˜50 μM, while MSPs less prone to aggregation can be produced effectively also at higher concentrations (˜100 μM—FIG. 3 and Table 2). In all cases, the reaction is effectively completed within 24 h at 37° C. (FIG. 13).

The disulfide-stabilized peptide-autocyclases: The second class of molecules that we tested were the naturally occurring cyclic peptides. These peptides feature head-to-tail circularization and are further stabilized by disulfide bonds. Here, we have several well-characterized peptides including SFTI (one disulfide bond)³⁷, Vc1.1 (two disulfide bonds)³⁸ and KalataB1 (kB1—three disulfide bonds)³⁹. We note that in these reactions the proteins remain monomeric in solution and thus do not require addition of detergents.

Initially, the autocyclase-L1b construct was used (as for the MSPs), but we found very slow rates of cyclisation reaction using this construct (>3 days for SFTI and kB1). Thus, we generated a series of autocyclase-L2a constructs. We find that for SFTI-L2a this significantly improves the reaction velocity and the reaction is complete within 24 h (at either 37° C. or room temperature). The L2a-kB1 reacts even faster and is completed within 3 h at 37° C. (or 4 h at room temperature). Since SFTI contains only one disulfide bond, the inclusion of GSH/GSSG in the buffer simply produces circularised, oxidized and natively folded SFTI (FIG. 4). Similarly, the circularised kalataB1 may be refolded into its native form using reported folding conditions.

It has been shown that SrtA mediated ligation is sensitive to the number of glycine residues at the N-terminus of the ligated protein, with higher numbers of glycines yielding improved cyclisation efficiency. In the case of the native sequence has been modified for improved oral stability⁴⁰. Thus, to maintain the length of linker used in this modified version, a GG-Vc1.1-L2a design was followed here²¹.

### The Mechanism of Autocyclase

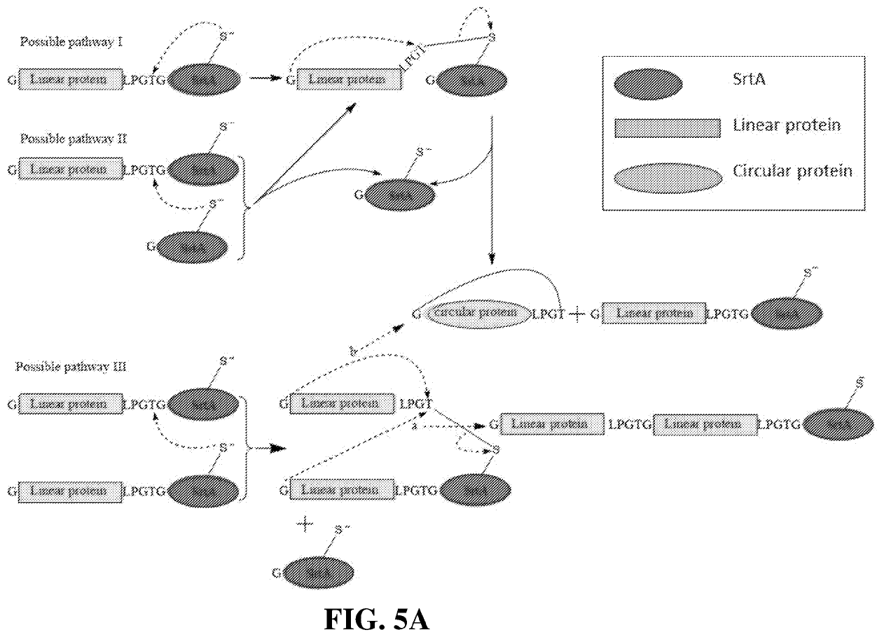

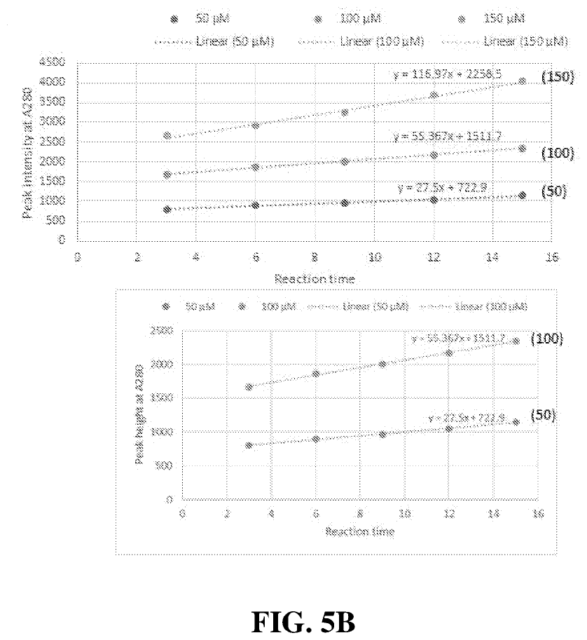

As noted above, the main motivation in generating the autocyclase enzyme was to change the reaction mechanism to overcome challenges associated with polymerisation in the bimolecular reaction. While the bimolecular reaction is an enzymatic reaction which often follows zero order kinetics, in this case the near stoichiometric amounts of enzyme used (i.e. in cMSP reactions reported), means that the reaction will more closely follow second order kinetics. Thus, while the unimolecular autocyclase reaction will be independent of the reactant concentration and the rate will scale linearly with the reactant concentration, in the bimolecular reaction the rate will scale by the square of the reactant concentration. However, since the product of the unimolecular reaction may catalyse subsequent reactions, it is expected that the reaction will deviate from first order kinetics once significant amounts of free sortase A is released. A summary of the different reaction pathways is shown in FIG. 5.

To characterise the reaction mechanism and kinetics we measured the initial reaction velocity (ν₀) for the L2a-kB1 autocyclase using three different starting concentrations. We find that at early time points the reaction rate doubles when the concentration of the L2a-kB1 autocyclase is doubled from 50 μm to 100 μM. This is consistent with first order reaction kinetics and confirms the proposed unimolecular reaction mechanism. When the concentration is further increased to 150 μM, the reaction rate increases by 4.35 times, compared to the reaction rate at 50 μM (ν₀^{1.33}) indicating that the reaction is predominantly the first order with contributions from a higher order reaction—consistent with the competition with bimolecular reactions (FIG. 5). Thus, we can conclude that, to good approximation, the reaction is first order at concentrations below 100 μM. This concentration range is similar, but slightly higher than what we find for the detergent assisted cyclisation of MSPs, suggesting that the interference from the bimolecular reaction will be dependent on the inherent oligomeric state of the target protein under the specific cyclisation conditions.

### Discussion

Nanodiscs (NDs) provide a physiologically relevant bilayer environment for performing biochemical and biophysical characterization of membrane proteins, with applications in a wide variety of fields⁴¹. In the pioneering work in establishing cyclised MSPs, the reaction yielded undesired byproducts²². Subsequently these polymeric byproducts were effectively suppressed by detergent assisted cyclisation and a dropwise addition of the MSP to the eSrtA solution³⁶. Johansen et al. further engineered a solubility-enhanced cMSP with improved production yields, by introduction of a high abundance of negatively charged amino acids.⁴⁶ However, both methods still require the low concentration of MSP to suppress the undesired polymeric by-products.

Furthermore, the high molar ratio of eSrtA to MSP used in these experiments requires an extra step for separate preparation of large amounts of eSrtA.

Recently, an alternative approach was introduced for the generation of cMSPs based on in vivo split intein ligation in E. coli.⁴⁷ Although the intein method eliminates the additional in vitro enzymatic reaction, there are extra purification steps required, while it is also necessary to introduce a His₆-tag and extra cysteine residue into the final cMSP sequence. The presence of a free cysteine residue further complicates application of this methodology to disulfide-bond containing proteins such as the cyclic peptides described here.

The autocyclase method described herein produces circular MSPs that are identical in sequence to those originally presented. The unimolecular reaction design results in higher yields, less reaction steps and reduced time (<two days including protein expression and purification). We also demonstrate the versatility of the method by producing a wide range of cMSP of varied lengths, including cMSP6, cMSP7, cMSP9, cMSP11, and cMSP20—this includes the first reports of a cysteine- and His-tag-free cMSP6 and cMSP7. Further, the presented procedure allows for purification of reactive sortase A as a byproduct. Finally, we note that previous studies have noted significant amounts of insoluble MSP9 and MSP11 after cell lysis, while the original MSP30 and MSP50 were purified under denaturing conditions. Here SrtA appears to function as a solubility enhancement tag and we find that all of the expressed MSP9, MSP11, and MSP20 are in the soluble fraction upon cell lysis²². The autocyclase approach, when applied to MSPs, therefore represents a significant improvement of the current nanodisc technology, and may facilitate increased uptake and utility of this powerful technology.

TABLE 1
Primers used in this study.

| Primer Name | Primer Sequence |
|---|---|
| P1-forward with KpnI site | CGG GAATCC GGTACC CAA GCTAAACCTCAAATT CCGAAAG |
| underlined, for wt-SrtA | |
| P1-reverse with XhoI site | TTTTTT CCG CTCGAG TTT GAC TTC TGT AGC TAC AAA GAT |
| underlined | TTT ACG |
| P2-forward, for Gly2Ala | TATACATATGgctTCGTCCCACCATCAC |
| P2-reverse | TCTCCTTCTTAAAGTTAAACAAAATTATTTC |
| P3-forward with NdeI site | CGCGGATCC CAT ATG GCT AGC AGC GAA AAC CTG TAT TTT |
| underlined, for MSP11 | CAG GGC AGC ACC |
| P3-reverse with KpnI site | GGCGAATTC GGT ACC CGG CAG CTG GGT G |
| underlined | |
| P4-forward, for removing | GAGAATTTGTACTTCCAAGGATC |
| N-terminal His-tag | |
| P4-reverse | GGACGAAGCCATATGTATATC |
| P5-forward, for introducing | catcatTGAGATCCGGCTGCTAAC |
| His₄ into His₆ at the c- | |
| terminal | |
| P5-reverse | atgatgGTGGTGGTGGTGGTGGTG |
| P6-forward, for introducing | tgggtccggtggtagtggtgggagtCAAGCTAAACCGCAGATC |
| (GGS)x5 linker in MSP9- | |
| eSrtA | |
| P6-reverse | cctgaacctcccgatcccccggtaccAGGCAGTTGTGTGTTAAG |
| P7-forward, for introducing | tgggtccggtggtagtggtgggagtCAAGCTAAACCTCAAATTC |
| (GGS)x5 linker in MSP9- | |
| wtSrtA | |
| P7-reverse = P6-reverse | cctgaacctcccgatcccccggtaccAGGCAGTTGTGTGTTAAG |
| P8-forward, for deleteing | TTGGGGGAGGAGATGCGT |
| H4 in MSP9 to make MSP7 | |
| P8-reverse | GGGTTGTACCTTAGCCTTCAC |
| P9-forward, for deleteing | TATAGTGATGAGTTGCGC |
| H4 and H6 in MSP9 to | |
| make MSP6 | |
| P9-reverse | GGGTTGTACCTTAGCCTTC |
| P10-forward, for | cgatgccGAGAATTTGTACTTCCAAGG |
| introducing an inhibitor | |
| peptide | |
| P10-reverse | cgtggcaaGGACGAAGCCATATGTATATC |
| P11-forward, optimized | cgcggaaaacctgtattttcagGGATCGACGTTTTCCAAG |
| inhibitory peptide, option1 | |
| P11-reverse | tcacgaggtaaagaactggccatATGTATATCTCCTTCTTAAAGTTAAAC |
| P12-forward, optimized | cgcagagaatttgtatttccagGGATCGACGTTTTCCAAG |
| inhibitory peptide, option2 | |
| P12-reverse | tcgcgtggaagggaggaagccatATGTATATCTCCTTCTTAAAGTTAAAC |
| P13-forward, for | gcgcagcCAAGCTAAACCTCAAATTCC |
| introducing a Thrombin site | |
| between | |
| LPGTGAAALEGT linker | |
| and SrtA | |
| P13-reverse | ggcaccagGGTACCCTCTAAAGCTGC |
| P14-forward, for | gcgctccCAAGCTAAACCTCAAATTCCGAAAG |
| introducing a Thrombin site | |
| between LPGTG(GGS)5 | |
| linker and SrtA | |
| P14-reverse | ggcacgagACTCCCACCACTACCACC |
| P15-forward, for removing | GAAAACCTGTATTTTCAGGG |
| N-terminal his tag in | |
| MSP11- | |
| LPGTGAAALEGTLVPRS- | |
| SrtA-His₁₀ | |
| P15-reverse | GCTGCTAGCCATATGTATATC |
| P16-forward, for | AAGAAGGAGATATAcatatg gccagttct |
| amplification of MSP20 and | gaaaacctgtattttcagggatcgacg |
| replace MSP9 in MSP9- | |
| LPGTGAAALEGTLVPRS- | |
| wtSrtA-His₁₀ | |
| P16-reverse | CAC CAG GGT ACC CTC TAA AGC TGC AGC ACC TGT ACC |
| | AGG TAA CTG TGT ATT TAA CTT TTT AGT ATA TTC TTC |
| P17-forward, for generating | CTGCCTGGTACCGGGGGA |
| empty autocyclase-L2a | |
| vector | |
| P17-reverse | TCCCTGAAAATACAGGTTTTCCGCG |
| P18-forward, to generate | gccgatttgctttccggatCTGCCTGGTACCGGGGGA |
| autocyclase-L2a-G-SFTI | |
| P18-reverse | ggaatgcttttggtgcagcgTCCCTGAAAATACAGGTTTTCCGCG |
| P19-forward, to generate | Cacctgcagctggccggtgtgcacccgcaacggcctgccggtg |
| autocyclase-L2a-G- | ACCGGGGGATCGGGAGGT |
| KalataB1 | |
| P19-reverse | Cagcccggggtgttgcaggtgccgcccacgcaggtttcgccgca |
| | TCCCTGAAAATACAGGTTTTCCGCG |
| P20-forward, to generate | gccgatttgctttccggatCTGCCTGGCACAGGTGCT |
| autocyclase-L1b-G-SFTI | |
| P20-reverse | ggaatgcttttggtgcagcgTCCTTGGAAGTACAAATTCTCGGAC |
| P21-forward, to generate | tccgccgatttgctttccggatCTGCCTGGCACAGGTGCT |
| autocyclase-L1b-GGG- | |
| SFTI | |
| P21-reverse | atgcttttggtgcagcggccaccTCCTTGGAAGTACAAATTCTCGGAC |
| P22-forward, to generate | tgggtccggtggtagtggtgggagtCTGGTGCCGCGCAGCCAA |
| autocyclase-L2a-GGG-SFTI | |
| P22-reverse | cctgaacctcccgatcccccggtaccAGGCAGATCCGGAAAGCAAATCGG |
| P23-forward, to generate | ggtggcTGCGGCGAAACCTGCGTG |
| autocyclase-L1b-GGG-kB1 | |
| P23-reverse | TCCTTGGAAGTACAAATTCTCGGACG |
| P24-forward, to generate | gtccggtggtagtggtgggagtCTGGTGCCGCGCAGCCAA |
| autocyclase-L2a-GGG-kB1 | |
| P24-reverse | ccacctgaacctcccgatcccccTGTCACAGGCAGGCCGTTG |
| P25-forward, to generate | ctatgatcatccggaaatttgcggtCTGCCTGGCACAGGTGCT |
| autocyclase-L1b-GG-Vc1.1 | |
| P25-reverse | ttgcagcgcggatcgctgcagcaaccTCCTTGGAAGTACAAATTCTCGGAC |
| P26-forward, to generate | tgggtccggtggtagtggtgggagtCTGGTGCCGCGCAGCCAA |
| autocyclase-L2a-GG-Vc1.1 | |
| P26-reverse | cctgaacctcccgatcccccggtaccAGGCAGACCGCAAATTTCCGG |

TABLE 2
The average yields of cMSP of various sizes per liter of
E. coli culture from srtA-fusion based circularisation.

| | Yield of | Yield of circular MSP |
|---|---|---|
| Constructs | fusion(mg/400 mL) | (cMSP)(mg/400 mL) |
| cMSPΔH4-6 (cMSP6) | | 2.2 |
| cMSPΔH4-5 (cMSP7) | | 2.1 |
| cMSPΔH5 (cMSP9) | 25.0 | 6.7 |
| cMSPD1 (cMSP11) | 19.6 | 8.4 |
| cMSP2N2 (cMSP20)* | 22.9 | 6.9 |

*The yield of MSP20 is heavily underestimated, since the amount of Ni-NTA is not enough to capture MSP20-sortase. Half of MSP20-sortase is estimated to have been lost.

TABLE 3
The characterization of circular and intact
MSP proteins by mass spectrometry.

| | Mass, Da | Mass, Da |
|---|---|---|
| | Circularised | Circularised |
| Constructs | (calculated) | (Observed) |
| cMSPΔH4-6 (cMSP6) | 14,456.37 | 14456 |
| cMSPΔH4-5 (cMSP7) | 16,953.20 | 16953 |
| cMSPΔH5 (cMSP9) | | |
| cMSPD1 (cMSP11) | | |
| cMSP2N2 (cMSP15) | | |

TABLE 4
Kinetic parameters of SrtA mutants with a reduced
activity compared to the autocyclase enzyme.

| | K_{cat}, | K_{m LPETG} | K_{cat}/K_{m LPETG} | Activity loss |
|---|---|---|---|---|
| Mutants | [s⁻¹] | [mM] | [M⁻¹S⁻¹] | [fold] |
| WT | 1.10 ± 0.06 | 8.76 ± 0.78 | 125 ± 18 | |
| V168A | 0.15 ± 0.01 | 6.56 ± 0.64 | 22.7 ± 3.6 | 5.5 |
| L169A | 1.23 × 10⁻² ± | 9.14 ± 0.15 | 1.35 ± 0.14 | 93 |
| | 0.06 × 10⁻² | | | |
| E171A | 0.16 ± 0.01 | 6.74 ± 0.69 | 23.1 ± 3.6 | 5.4 |
| Q172A | 1.13 ± 0.11 | 12.7 ± 1.9 | 89.3 ± 22 | 1.4 |
| R197A | 6.28 × 10⁻⁴ ± | 4.69 ± 0.12 | 0.13 ± 0.01 | 960 |
| | 0.60 × 10⁻⁵ | | | |
| R197K | 1.90 × 10⁻³ ± | 10.4 ± 0.19 | 0.18 ± 0.01 | 690 |
| | 0.20 × 10⁻⁴ | | | |

### REFERENCES

1. Cascales, L. & Craik, D. J. Naturally occurring circular proteins: distribution, biosynthesis and evolution. Org. Biomol. Chem. 8, 5035-5047 (2010).
2. Clark, R. J., Akcan, M., Kaas, Q., Daly, N. L. & Craik, D. J. Cyclization of conotoxins to improve their biopharmaceutical properties. Toxicon (2010).
3. Wong, C. T. T. et al. Orally Active Peptidic Bradykinin B1 Receptor Antagonists Engineered from a Cyclotide Scaffold for Inflammatory Pain Treatment. Angewandte Chemie International Edition, n/a-n/a (2012).
4. Driggers, E. M., Hale, S. P., Lee, J. & Terrett, N. K. The exploration of macrocycles for drug discovery—an underexploited structural class. Nat. Rev. Drug Discov. 7, 608-624 (2008).
5. Dawson, P. E., Muir, T. W., Clark-Lewis, I. & Kent, S. B. Synthesis of proteins by native chemical ligation. Science 266, 776-779 (1994).
6. Muir, T. W. Semisynthesis of proteins by expressed protein ligation. Annu. Rev. Biochem. 72, 249-289 (2003).
7. Muir, T. W., Sondhi, D. & Cole, P. A. Expressed protein ligation: a general method for protein engineering. Proc. Natl. Acad. Sci. U.S.A 95, 6705-6710 (1998).
8. Kimura, R. & Camarero, J. A. Expressed protein ligation: a new tool for the biosynthesis of cyclic polypeptides. Protein Pept. Lett. 12, 789-794 (2005).
9. Kimura, R. H., Tran, A. T. & Camarero, J. A. Biosynthesis of the cyclotide Kalata B1 by using protein splicing. Angew. Chem. Int. Ed. Engl. 45, 973-976 (2006).
10. Tavassoli, A. & Benkovic, S. J. Split-intein mediated circular ligation used in the synthesis of cyclic peptide libraries in E. coli. Nat. Protoc. 2, 1126-1133 (2007).
11. Kawakami, T. et al. Diverse backbone-cyclized peptides via codon reprogramming. Nat. Chem. Biol. 5, 888-890 (2009).
12. Nguyen, G. K. T. et al. Butelase 1 is an Asx-specific ligase enabling peptide macrocyclization and synthesis. Nat. Chem. Biol. 10, 732-738 (2014).
13. Harris, K. S. et al. Efficient backbone cyclization of linear peptides by a recombinant asparaginyl endopeptidase. Nat. Commun. 6, 10199 (2015).
14. Yang, R. et al. Engineering a Catalytically Efficient Recombinant Protein Ligase. J Am. Chem. Soc. 139, 5351-5358 (2017).
15. Lee, J., McIntosh, J., Hathaway, B. J. & Schmidt, E. W. Using marine natural products to discover a protease that catalyzes peptide macrocyclization of diverse substrates. J Am. Chem. Soc. 131, 2122-2124 (2009).
16. Barber, C. J. et al. The two-step biosynthesis of cyclic peptides from linear precursors in a member of the plant family Caryophyllaceae involves cyclization by a serine protease-like enzyme. J. Biol. Chem. 288, 12500-12510 (2013).
17. Luo, H. et al. Peptide macrocyclization catalyzed by a prolyl oligopeptidase involved in alpha-amanitin biosynthesis. Chem. Biol. 21, 1610-1617 (2014).
18. Pi, N. et al. Recombinant Butelase-Mediated Cyclization of the p53-Binding Domain of the Oncoprotein MdmX-Stabilized Protein Conformation as a Promising Model for Structural Investigation. Biochemistry 58, 3005-3015 (2019).
19. Mazmanian, S. K., Liu, G., Ton-That, H. & Schneewind, O. Staphylococcus aureus sortase, an enzyme that anchors surface proteins to the cell wall. Science 285, 760-763 (1999).
20. Antos, J. M. et al. Site-specific N- and C-terminal labeling of a single polypeptide using sortases of different specificity. J Am. Chem. Soc. 131, 10800-10801 (2009).
21. Jia, X. et al. Semienzymatic cyclization of disulfide-rich peptides using Sortase A. J Biol. Chem. 289, 6627-6638 (2014).
22. Nasr, M. L. et al. Covalently circularized nanodiscs for studying membrane proteins and viral entry. Nat. Methods 14, 49-52 (2017).
23. Suree, N. et al. The structure of the Staphylococcus aureus sortase-substrate complex reveals how the universally conserved LPXTG sorting signal is recognized. J. Biol. Chem. 284, 24465-24477 (2009).
24. Jacobitz, A. W., Kattke, M. D., Wereszczynski, J. & Clubb, R. T. Sortase Transpeptidases: Structural Biology and Catalytic Mechanism. Adv Protein Chem Struct Biol 109, 223-264 (2017).
25. Zhou, C., Yan, Y., Fang, J., Cheng, B. & Fan, J. A new fusion protein platform for quantitatively measuring activity of multiple proteases. Microb. Cell Fact. 13, 44 (2014).
26. Warden-Rothman, R., Caturegli, I., Popik, V. & Tsourkas, A. Sortase-tag expressed protein ligation: combining protein purification and site-specific bioconjugation into a single step. Anal. Chem. 85, 11090-11097 (2013).
27. Wang, J. et al. Oligopeptide Targeting Sortase A as Potential Anti-infective Therapy for Staphylococcus aureus. Front Microbiol 9, 245 (2018).
28. Mao, H. Y. A self-cleavable sortase fusion for one-step purification of free recombinant proteins. Protein Expr. Purif. 37, 253-263 (2004).
29. Jia, X., Crawford, T., Zhang, A. H. & Mobli, M. A new vector coupling ligation-independent cloning with sortase a fusion for efficient cloning and one-step purification of tag-free recombinant proteins. Protein Expr. Purif. (2019).
30. Chen, I., Dorr, B. M. & Liu, D. R. A general strategy for the evolution of bond-forming enzymes using yeast display. Proc. Natl. Acad. Sci. U.S.A 108, 11399-11404 (2011).
31. Ben-Bassat, A. et al. Processing of the initiation methionine from proteins: properties of the Escherichia coli methionine aminopeptidase and its gene structure. J. Bacteriol. 169, 751-757 (1987).
32. Gangola, P. & Rosen, B. P. Maintenance of intracellular calcium in Escherichia coli. J. Biol. Chem. 262, 12570-12574 (1987).
33. Faas, R. et al. Time-course and degradation rate of membrane scaffold protein (MSP1D1) during recombinant production. Biotechnol Rep (Amst) 17, 45-48 (2018).
34. Mei, X. & Atkinson, D. Crystal structure of C-terminal truncated apolipoprotein A-I reveals the assembly of high density lipoprotein (HDL) by dimerization. J Biol. Chem. 286, 38570-38582 (2011).
35. Zhang, A. H. et al. Elucidating the Lipid Binding Properties of Membrane-Active Peptides Using Cyclised Nanodiscs. Frontiers in Chemistry 7 (2019).
36. Yusuf, Y. et al. Optimization of the Production of Covalently Circularized Nanodiscs and Their Characterization in Physiological Conditions. Langmuir 34, 3525-3532 (2018).
37. Luckett, S. et al. High-resolution structure of a potent, cyclic proteinase inhibitor from sunflower seeds. J Mol. Biol. 290, 525-533 (1999).
38. Sandall, D. et al. A novel α-conotoxin identified by gene sequencing is active in suppressing the vascular response to selective stimulation of sensory nerves in vivo. Biochemistry 42, 6904-6911 (2003).
39. Saether, O. et al. Elucidation of the primary and three-dimensional structure of the uterotonic polypeptide kalata B1. Biochemistry 34, 4147-4158 (1995).
40. Clark, R. J. et al. The engineering of an orally active conotoxin for the treatment of neuropathic pain. Angew. Chem. Int. Ed. Engl. 49, 6545-6548 (2010).
41. Denisov, I. G. & Sligar, S. G. Nanodiscs in Membrane Biochemistry and Biophysics. Chem. Rev. 117, 4669-4713 (2017).
42. Denisov, I. G., Grinkova, Y. V., Lazarides, A. A. & Sligar, S. G. Directed self-assembly of monodisperse phospholipid bilayer Nanodiscs with controlled size. J. Am. Chem. Soc. 126, 3477-3487 (2004).
43. Hagn, F., Etzkorn, M., Raschle, T. & Wagner, G. Optimized Phospholipid Bilayer Nanodiscs Facilitate High-Resolution Structure Determination of Membrane Proteins. J. Am. Chem. Soc. 135, 1919-1925 (2013).
44. Hagn, F. & Wagner, G. Structure refinement and membrane positioning of selectively labeled OmpX in phospholipid nanodiscs. J. Biomol. NMR 61, 249-260 (2015).
45. Raschle, T. et al. Structural and functional characterization of the integral membrane protein VDAC-1 in lipid bilayer nanodiscs. J Am. Chem. Soc. 131, 17777-17779 (2009).
46. Johansen, N. T. et al. Circularized and solubility-enhanced MSPs facilitate simple and high yield production of stable nanodiscs for studies of membrane proteins in solution. FEBS J (2019).
47. Miehling, J., Goricanec, D. & Hagn, F. A Split-Intein-Based Method for the Efficient Production of Circularized Nanodiscs for Structural Studies of Membrane Proteins. ChemBioChem 19, 1927-1933 (2018).
48. Jennings, M. J., Barrios, A. F. & Tan, S. Elimination of truncated recombinant protein expressed in Escherichia coli by removing cryptic translation initiation site. Protein Expr. Purif. 121, 17-21 (2016).
49. Whitaker, W. R., Lee, H., Arkin, A. P. & Dueber, J. E. Avoidance of truncated proteins from unintended ribosome binding sites within heterologous protein coding sequences. ACS Synth Biol 4, 249-257 (2015).
50. Lawrence, A.-M. & Besir, H. Staining of proteins in gels with Coomassie G-250 without organic solvent and acetic acid. JoVE (Journal of Visualized Experiments), e1350 (2009).
51. Ritchie, T. K. et al. Chapter 11—Reconstitution of membrane proteins in phospholipid bilayer nanodiscs. Methods in Enzymology 464, 211-231 (2009).
52. Janosi, L. & Gorfe, A. A. Simulating POPC and POPC/POPG Bilayers: Conserved Packing and Altered Surface Reactivity. Journal of Chemical Theory and Computation 6, 3267-3273 (2010).

1. (canceled)

2. A fusion protein capable of producing a circularised form of a target protein, the fusion protein comprising:
the target protein;
at least one circularisation site adjacent the target protein;
at least one inhibitory domain, and
an enzyme domain capable of interacting with the at least one circularisation site and circularising the target protein.

3. The fusion protein of claim 2, wherein the target protein is circularised by way of a unimolecular reaction.

4. The fusion protein of claim 2, wherein the fusion protein integrates protein expression, purification and circularisation into one single molecule.

5. (canceled)

6. The fusion protein of claim 2, wherein the enzyme domain comprises at least one ligase or cyclase, or an enzymatically active fragment, variant or derivative thereof.

7. The fusion protein of claim 6, wherein the at least one circularisation site comprises first and second circularisation sites adjacent respective terminal ends of the target protein.

8. The fusion protein of claim 6, wherein the enzyme domain is a sortase or an asparaginyl endopeptidase (AEP), or a combination thereof, or an enzymatically active fragment, variant or derivative thereof.

9. The fusion protein of claim 6, further comprising at least one spacer, wherein preferably the at least one spacer is situated between the target protein and the enzyme domain.

10. The fusion protein of claim 9, wherein the at least one inhibitory domain is adjacent one or more of the at least one circularisation site, and optionally the at least one inhibitory domain is positioned at, near, adjacent or towards an N- or C-terminus of the fusion protein.

11. The fusion protein of claim 2, wherein the circularised form of the target protein is capable of binding to a target of interest, such as a therapeutic target or pesticide target, wherein preferably the target of interest is a biomacromolecule, such as a protein, a peptide, a nucleic acid, a polycarbohydrate, or a small molecule such as an organic compound or an organometallic complex, or any other molecule that contributes to a disease or is a target of a pesticide.

12. The fusion protein of claim 2, wherein the target protein is or comprises a membrane scaffold protein (MSP), or a fragment, variant or derivative thereof, wherein preferably a circularised MSP or fragment, variant or derivative thereof is capable of being used in the production of a nanodisc.

13. The fusion protein of claim 2, wherein the target protein is or comprises a cyclotide, or a fragment, variant or derivative thereof, wherein preferably the cyclotide is SFTI, Vc1.1, Kalata B1 or MCOTI-II, or an orthologue, fragment, variant or derivative thereof.

14-16. (canceled)

17. An isolated nucleic acid [1] encoding the fusion protein of claim 2; a genetic construct [2] comprising said nucleic acid [1]; or, a host cell comprising said nucleic acid [1] and/or said genetic construct [2].

18. (canceled)

19. A method for circularising a target protein, said method including the steps of:
(a) providing a fusion protein comprising:
a target protein;
at least one circularisation site adjacent the target protein;
at least one inhibitory domain,
an enzyme domain capable of circularising the target protein; and
optionally a spacer positioned between the target protein and the enzyme domain; and
(b) facilitating interaction of the enzyme domain with the at least one circularisation site to thereby circularise the amino acid sequence of the target protein.

20-21. (canceled)

22. The method of claim 19, comprising the step of modulating activity of the enzyme domain by introducing at least one mutation into the enzyme domain, wherein modulated activity results in: i) increased or reduced catalytic activity; ii) increased or reduced binding to the at least one circularisation site; and/or iii) an altered circularisation site.

23. The method of claim 19, wherein the target protein of the fusion protein is circularised by way of an enzyme domain released from a like said fusion protein.

24. The method of claim 19:
(i) further including an initial step of producing the fusion protein;
(ii) wherein the step of facilitating interaction of the enzyme domain with the at least one circularisation site comprises the step of activating the enzyme domain;
(iii) wherein the step of facilitating interaction of the enzyme domain with the at least one circularisation site comprises the step of removing the inhibitory domain adjacent one or more of the at least one circularisation site;
(iv) further including a subsequent step of removing the spacer from the enzyme domain;
(v) further including a subsequent step of isolating or purifying the enzyme domain removed from the fusion protein by way of an affinity tag positioned at or towards an N- or C-terminus of the fusion protein and adjacent the enzyme domain.

25. (canceled)


SEQUENCE LISTING (142 sequences)

SEQ ID NO: 1 - protein, 351 aa, Artificial Sequence
  Synthetic sequence
MGSSHHHHHHENLYFQGSTFSKLREQLGPVTQEFWDNLEKETEGLRQEMSKDLEEVKAKVQPYLDDFQKKWQEEMELYRQKVEPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGAAALEGTQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 2 - protein, 356 aa, Artificial Sequence
  Synthetic sequence
MASSENLYFQGSTFSKLREQLGPVTQEFWDNLEKETEGLRQEMSKDLEEVKAKVQPYLDDFQKKWQEEMELYRQKVEPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 3 - protein, 378 aa, Artificial Sequence
  Synthetic sequence
MASSENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 4 - protein, 558 aa, Artificial Sequence
  Synthetic sequence
MASSENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 5 - protein, 334 aa, Artificial Sequence
  Synthetic sequence
MASSENLYFQGSTFSKLREQLGPVTQEFWDNLEKETEGLRQEMSKDLEEVKAKVQPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 6 - protein, 312 aa, Artificial Sequence
  Synthetic sequence
MASSENLYFQGSTFSKLREQLGPVTQEFWDNLEKETEGLRQEMSKDLEEVKAKVQPYSDELRQRLAARLEALKENGGARLAEYHAKATEHLSTLSEKAKPALEDLRQGLLPVLESFKVSFLSALEEYTKKLNTQLPGTGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 7 - protein, 354 aa, Artificial Sequence
  Synthetic sequence
MASSENLYFQGSTFSKLREQLGPVTQEFWDNLEKETEGLRQEMSKDLEEVKAKVQPYLDDFQKKWQEEMELYRQKVEPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 8 - protein, 332 aa, Artificial Sequence
  Synthetic sequence
MASSENLYFQGSTFSKLREQLGPVTQEFWDNLEKETEGLRQEMSKDLEEVKAKVQPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 9 - protein, 310 aa, Artificial Sequence
  Synthetic sequence
MASSENLYFQGSTFSKLREQLGPVTQEFWDNLEKETEGLRQEMSKDLEEVKAKVQPYSDELRQRLAARLEALKENGGARLAEYHAKATEHLSTLSEKAKPALEDLRQGLLPVLESFKVSFLSALEEYTKKLNTQLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 10 - protein, 376 aa, Artificial Sequence
  Synthetic sequence
MASSENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 11 - protein, 556 aa, Artificial Sequence
  Synthetic sequence
MASSENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 12 - protein, 212 aa, Artificial Sequence
  Synthetic sequence
MASSLPRDAENLYFQGRCTKSIPPICFPDLPGTGGSGGSGGSGGSGGSLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 13 - protein, 225 aa, Artificial Sequence
  Synthetic sequence
MASSLPRDAENLYFQGCGETCVGGTCNTPGCTCSWPVCTRNGLPVTGGSGGSGGSGGSGGSLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 14 - protein, 200 aa, Artificial Sequence
  Synthetic sequence
MASSENLYFQGRCTKSIPPICFPDLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 15 - protein, 202 aa, Artificial Sequence
  Synthetic sequence
MASSENLYFQGGGGRCTKSIPPICFPDLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHH

SEQ ID NO: 16 - protein, 209 aa, Artificial Sequence
  Synthetic sequence
MASSENLYFQGGGRCTKSIPPICFPDLPGTGGSGGSGGSGGSGGSLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 17 - protein, 222 aa, Artificial Sequence
  Synthetic sequence
MASSENLYFQGGGCGETCVGGTCNTPGCTCSWPVCTRNGLPVTGGSGGSGGSGGSGGSLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 18 - protein, 215 aa, Artificial Sequence
  Synthetic sequence
MASSENLYFQGGGCGETCVGGTCNTPGCTCSWPVCTRNGLPVTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 19 - protein, 213 aa, Artificial Sequence
  Synthetic sequence
MASSENLYFQGCGETCVGGTCNTPGCTCSWPVCTRNGLPVTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 20 - protein, 204 aa, Artificial Sequence
  Synthetic sequence
MASSENLYFQGGCCSDPRCNYDHPEICGLPGTGAAALEGTLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 21 - protein, 211 aa, Artificial Sequence
  Synthetic sequence
MASSENLYFQGGCCSDPRCNYDHPEICGLPGTGGSGGSGGSGGSGGSLVPRSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVKLEHHHHHHHHHH

SEQ ID NO: 22 - DNA, 1056 bp, Artificial Sequence
  Synthetic sequence
atggggtcgtcccaccatcaccaccatcatgagaatttgtacttccaaggatcgacgttttccaagttacgcgaacagttaggacccgtaacgcaggaattctgggacaaccttgagaaagagacggaaggccttcgccaggagatgtcaaaagaccttgaggaagtgaaggctaaggtacaaccctatctggacgattttcaaaagaagtggcaagaagaaatggagttgtatcgtcaaaaagttgaacctttgggggaggagatgcgtgatcgcgcccgcgcccacgtggatgcattgcgcacgcatttagctccatatagtgatgagttgcgccagcgtttggccgcacgtttagaggctttgaaagagaatggcggtgcccgtctggccgagtaccatgcaaaggcgacagaacatttgtccaccttgagcgagaaagctaaaccggctctggaggacttgcgtcagggcttgcttccggtacttgaatcattcaaggtgtcctttctgtctgccttagaagagtatactaagaagcttaacacacaactgcctggcacaggtgctgcagctttagagggtacccaagctaaaccgcagatccccaaagacaaatctaaagttgcaggttatattgagatcccagacgcggatattaaggagcccgtgtatccgggtcccgccactcgcgagcagttgaatcgcggagtctcctttgcagaggaaaatgaatcgttggatgaccagaatatctctattgccggtcatacattcatcgaccgtccaaattaccaattcactaaccttaaagccgcgaaaaaggggtcgatggtctatttcaaggtgggcaatgaaacacgcaaatataaaatgacttcgattcgtaacgtcaaaccaacggctgtggaagtgttagacgagcaaaaaggcaaggataagcaacttactttaattacgtgtgacgattataatgaagagacaggagtatgggagacacgcaaaatcttcgtggcgacggaggttaagctcgagcatcatcatcatcaccactag

SEQ ID NO: 23 - DNA, 1071 bp, Artificial Sequence
  Synthetic sequence
atggcttcgtccgagaatttgtacttccaaggatcgacgttttccaagttacgcgaacagttaggacccgtaacgcaggaattctgggacaaccttgagaaagagacggaaggccttcgccaggagatgtcaaaagaccttgaggaagtgaaggctaaggtacaaccctatctggacgattttcaaaagaagtggcaagaagaaatggagttgtatcgtcaaaaagttgaacctttgggggaggagatgcgtgatcgcgcccgcgcccacgtggatgcattgcgcacgcatttagctccatatagtgatgagttgcgccagcgtttggccgcacgtttagaggctttgaaagagaatggcggtgcccgtctggccgagtaccatgcaaaggcgacagaacatttgtccaccttgagcgagaaagctaaaccggctctggaggacttgcgtcagggcttgcttccggtacttgaatcattcaaggtgtcctttctgtctgccttagaagagtatactaagaagcttaacacacaactgcctggtaccgggggatcgggaggttcaggtgggtccggtggtagtggtgggagtcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 24 - DNA, 1137 bp, Artificial Sequence
  Synthetic sequence
atggctagcagcgaaaacctgtattttcagggcagcacctttagcaaactgcgtgaacagctgggcccggtgacccaggaattttgggataacctggaaaaagaaaccgaaggcctgcgtcaggaaatgagcaaagatctggaagaggtgaaagcgaaagtgcagccgtatctggatgactttcagaaaaaatggcaggaagagatggaactgtatcgtcagaaagtggaaccgctgcgtgcggaactgcaggaaggcgcgcgtcagaaactgcatgaactgcaggaaaaactgagcccgctgggcgaagagatgcgtgatcgtgcgcgtgcgcatgtggatgcgctgcgtacccatctggcgccgtatagcgatgaactgcgtcagcgtctggcggcccgtctggaagcgctgaaagaaaacggcggtgcgcgtctggcggaatatcatgcgaaagcgaccgaacatctgagcaccctgagcgaaaaagcgaaaccggcgctggaagatctgcgtcagggcctgctgccggtgctggaaagctttaaagtgagctttctgagcgcgctggaagagtataccaaaaaactgaacacccagctgccgggtaccgggggatcgggaggttcaggtgggtccggtggtagtggtgggagtcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 25 - DNA, 1677 bp, Artificial Sequence
  Synthetic sequence
atggcatcgtcggagaacttgtatttccaaggctctactttctcgaagttgcgtgagcagttgggacctgtgacacaagagttctgggataatttagaaaaggagacagaagggctgcgtcaagagatgagtaaagaccttgaagaagttaaagcaaaggtgcagccctatctggatgatttccaaaaaaaatggcaagaagaaatggaattataccgtcagaaggtagagccacttcgtgcagaattgcaagaaggcgcacgccagaagttgcacgaactgcaagaaaaattgtcacctttgggggaggagatgcgcgaccgtgcacgcgcgcacgttgacgccttgcgtacgcatctggcgccgtactctgacgaattacgtcagcgcttggccgcgcgcttagaggccttgaaggagaacgggggagcgcgtcttgcagagtaccatgccaaagccacggaacatctgtccaccttgagcgagaaggcgaagccagcactggaagacttacgccagggtttgctgccagtccttgagtcttttaaagtatcgtttctttctgcgcttgaggaatacacgaagaagttaaacactcagggtactccagtgacacaggagttttgggataatttggaaaaagagactgaagggcttcgccaagagatgtcgaaggatttggaagaggtaaaggcgaaggtccaaccttacctggacgatttccaaaagaagtggcaggaagaaatggagttataccgtcagaaagtcgaacctttacgtgccgaattacaagaaggagcacgccaaaaacttcatgagcttcaggagaagctgtccccccttggtgaggagatgcgcgaccgtgcgcgtgctcatgtagatgcattacgtacccaccttgccccctatagcgatgagttgcgtcagcgtcttgccgcccgcctggaagctttgaaagagaatggcggtgctcgtttagcagagtatcacgccaaggccaccgaacatctttcaactttgtctgagaaagccaaacctgcgttagaagacttgcgtcaagggcttctgcctgtcttagagtcgttcaaggtgtcatttctgtcggcgcttgaagaatatactaaaaagttgaatacacagttacctggtaccgggggatcgggaggttcaggtgggtccggtggtagtggtgggagtcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 26 - DNA, 1005 bp, Artificial Sequence
  Synthetic sequence
atggcttcgtccgagaatttgtacttccaaggatcgacgttttccaagttacgcgaacagttaggacccgtaacgcaggaattctgggacaaccttgagaaagagacggaaggccttcgccaggagatgtcaaaagaccttgaggaagtgaaggctaaggtacaacccttgggggaggagatgcgtgatcgcgcccgcgcccacgtggatgcattgcgcacgcatttagctccatatagtgatgagttgcgccagcgtttggccgcacgtttagaggctttgaaagagaatggcggtgcccgtctggccgagtaccatgcaaaggcgacagaacatttgtccaccttgagcgagaaagctaaaccggctctggaggacttgcgtcagggcttgcttccggtacttgaatcattcaaggtgtcctttctgtctgccttagaagagtatactaagaagcttaacacacaactgcctggtaccgggggatcgggaggttcaggtgggtccggtggtagtggtgggagtcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 27 - DNA, 939 bp, Artificial Sequence
  Synthetic sequence
atggcttcgtccgagaatttgtacttccaaggatcgacgttttccaagttacgcgaacagttaggacccgtaacgcaggaattctgggacaaccttgagaaagagacggaaggccttcgccaggagatgtcaaaagaccttgaggaagtgaaggctaaggtacaaccctatagtgatgagttgcgccagcgtttggccgcacgtttagaggctttgaaagagaatggcggtgcccgtctggccgagtaccatgcaaaggcgacagaacatttgtccaccttgagcgagaaagctaaaccggctctggaggacttgcgtcagggcttgcttccggtacttgaatcattcaaggtgtcctttctgtctgccttagaagagtatactaagaagcttaacacacaactgcctggtaccgggggatcgggaggttcaggtgggtccggtggtagtggtgggagtcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 28 - DNA, 1065 bp, Artificial Sequence
  Synthetic sequence
atggcttcgtccgagaatttgtacttccaaggatcgacgttttccaagttacgcgaacagttaggacccgtaacgcaggaattctgggacaaccttgagaaagagacggaaggccttcgccaggagatgtcaaaagaccttgaggaagtgaaggctaaggtacaaccctatctggacgattttcaaaagaagtggcaagaagaaatggagttgtatcgtcaaaaagttgaacctttgggggaggagatgcgtgatcgcgcccgcgcccacgtggatgcattgcgcacgcatttagctccatatagtgatgagttgcgccagcgtttggccgcacgtttagaggctttgaaagagaatggcggtgcccgtctggccgagtaccatgcaaaggcgacagaacatttgtccaccttgagcgagaaagctaaaccggctctggaggacttgcgtcagggcttgcttccggtacttgaatcattcaaggtgtcctttctgtctgccttagaagagtatactaagaagcttaacacacaactgcctggcacaggtgctgcagctttagagggtaccctggtgccgcgcagccaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 29 - DNA, 999 bp, Artificial Sequence
  Synthetic sequence
atggcttcgtccgagaatttgtacttccaaggatcgacgttttccaagttacgcgaacagttaggacccgtaacgcaggaattctgggacaaccttgagaaagagacggaaggccttcgccaggagatgtcaaaagaccttgaggaagtgaaggctaaggtacaacccttgggggaggagatgcgtgatcgcgcccgcgcccacgtggatgcattgcgcacgcatttagctccatatagtgatgagttgcgccagcgtttggccgcacgtttagaggctttgaaagagaatggcggtgcccgtctggccgagtaccatgcaaaggcgacagaacatttgtccaccttgagcgagaaagctaaaccggctctggaggacttgcgtcagggcttgcttccggtacttgaatcattcaaggtgtcctttctgtctgccttagaagagtatactaagaagcttaacacacaactgcctggcacaggtgctgcagctttagagggtaccctggtgccgcgcagccaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 30 - DNA, 933 bp, Artificial Sequence
  Synthetic sequence
atggcttcgtccgagaatttgtacttccaaggatcgacgttttccaagttacgcgaacagttaggacccgtaacgcaggaattctgggacaaccttgagaaagagacggaaggccttcgccaggagatgtcaaaagaccttgaggaagtgaaggctaaggtacaaccctatagtgatgagttgcgccagcgtttggccgcacgtttagaggctttgaaagagaatggcggtgcccgtctggccgagtaccatgcaaaggcgacagaacatttgtccaccttgagcgagaaagctaaaccggctctggaggacttgcgtcagggcttgcttccggtacttgaatcattcaaggtgtcctttctgtctgccttagaagagtatactaagaagcttaacacacaactgcctggcacaggtgctgcagctttagagggtaccctggtgccgcgcagccaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 31 - DNA, 1131 bp, Artificial Sequence
  Synthetic sequence
atggctagcagcgaaaacctgtattttcagggcagcacctttagcaaactgcgtgaacagctgggcccggtgacccaggaattttgggataacctggaaaaagaaaccgaaggcctgcgtcaggaaatgagcaaagatctggaagaggtgaaagcgaaagtgcagccgtatctggatgactttcagaaaaaatggcaggaagagatggaactgtatcgtcagaaagtggaaccgctgcgtgcggaactgcaggaaggcgcgcgtcagaaactgcatgaactgcaggaaaaactgagcccgctgggcgaagagatgcgtgatcgtgcgcgtgcgcatgtggatgcgctgcgtacccatctggcgccgtatagcgatgaactgcgtcagcgtctggcggcccgtctggaagcgctgaaagaaaacggcggtgcgcgtctggcggaatatcatgcgaaagcgaccgaacatctgagcaccctgagcgaaaaagcgaaaccggcgctggaagatctgcgtcagggcctgctgccggtgctggaaagctttaaagtgagctttctgagcgcgctggaagagtataccaaaaaactgaacacccagctgccgggtacgggcgccgctgcactggaaggtaccctggtgccgcgcagccaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 32 - DNA, 1671 bp, Artificial Sequence
  Synthetic sequence
atggccagttctgaaaacctgtattttcagggatcgacgttttccaagttacgtgagcagttaggacctgttacacaagagttctgggataacttagagaaagagacagaagggctgcgtcaagagatgagtaaagaccttgaagaagttaaagcaaaggttcagccctatctggatgatttccagaagaaatggcaggaggaaatggaattataccgtcagaaggtagagccacttcgtgcagaattgcaagaaggcgcacgccagaagttacacgaactgcaagaaaaattatcacctttaggggaggagatgcgcgaccgtgcacgcgcgcacgttgacgccttacgtacgcatctggcgccgtactctgacgaattacgtcagcgcttagccgcgcgcttagaggccttaaaggagaacgggggagcgcgtcttgcagagtaccatgccaaagccacggaacatctgtccaccttgagcgagaaggcgaagccagcactggaagacttacgccagggtttactgccagtccttgagtcttttaaagtatcgtttctttctgcgcttgaggaatacacgaagaagttaaacactcagggtactccagttacacaggagttttgggataatttagaaaaagagactgaagggcttcgccaagagatgtcgaaggatttagaagaggtaaaggcgaaggtccaaccttacctggacgatttccagaagaagtggcaagaagaaatggagttataccgtcagaaagtcgaacctttacgtgccgaattacaagaaggagcacgccaaaaacttcatgagcttcaggagaagctgtccccccttggtgaagagatgcgcgaccgtgcgcgtgctcatgtagatgcattacgtacccaccttgccccctatagcgatgagttacgtcagcgtcttgccgcccgcctggaagctttaaaagagaatggcggtgctcgtttagcagagtatcacgccaaggccaccgaacatctttcaactttatctgagaaagccaaacctgcgttagaagacttacgtcaagggcttctgcctgtcttagagtcgttcaaggtttcatttctgtcggcgcttgaagaatatactaaaaagttaaatacacagttacctggtacaggtgctgcagctttagagggtaccctggtgccgcgcagccaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 33 - DNA, 639 bp, Artificial Sequence
  Synthetic sequence
atggccagttctttacctcgtgacgcggaaaacctgtattttcagggacgctgcaccaaaagcattccgccgatttgctttccggatctgcctggtaccgggggatcgggaggttcaggtgggtccggtggtagtggtgggagtctcgtgccgcgctcccaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 34 - DNA, 678 bp, Artificial Sequence
  Synthetic sequence
atggccagttctttacctcgtgacgcggaaaacctgtattttcagggatgcggcgaaacctgcgtgggcggcacctgcaacaccccgggctgcacctgcagctggccggtgtgcacccgcaacggcctgccggtgaccgggggatcgggaggttcaggtgggtccggtggtagtggtgggagtctcgtgccgcgctcccaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaactcgagcaccaccaccaccaccaccatcatcatcattga

SEQ ID NO: 35 - protein, 12 aa, Artificial Sequence
  Synthetic sequence
GAAALEGTQAKP

SEQ ID NO: 36 - protein, 12 aa, Artificial Sequence
  Synthetic sequence
GGSGGSGGQAKP

SEQ ID NO: 37 - protein, 13 aa, Artificial Sequence
  Synthetic sequence
GAAALEGTLVPRS

SEQ ID NO: 38 - protein, 15 aa, Artificial Sequence
  Synthetic sequence
GGSGGSGGSGGSGGS

SEQ ID NO: 39 - protein, 20 aa, Artificial Sequence
  Synthetic sequence
GGSGGSGGSGGSGGSLVPRS

SEQ ID NO: 40 - protein, 18 aa, Artificial Sequence
  Synthetic sequence
  MISC_FEATURE (1)..(18): peptide is cyclised
GRCTKSIPPICFPDLPGT

SEQ ID NO: 41 - protein, 31 aa, Artificial Sequence
  Synthetic sequence
  MISC_FEATURE (1)..(31): peptide is cyclised
GCGETCVGGTCNTPGCTCSWPVCTRNGLPVT

SEQ ID NO: 42 - protein, 22 aa, Artificial Sequence
  Synthetic sequence
  MISC_FEATURE (1)..(22): peptide is cyclised
GGCCSDPRCNYDHPEICGLPGT

SEQ ID NO: 43 - DNA, 87 bp, Artificial Sequence
  Synthetic sequence
tctagaaataattttgtttaactttaagaaggagatatacatatggccagttctttacctcgtgacgcggaaaacctgtattttcag

SEQ ID NO: 44 - DNA, 87 bp, Artificial Sequence
  Synthetic sequence
tctagaaataattttgtttaactttaagaaggagatatacatatggccagttctctaccccgtgatgcggaaaacctgtattttcag

SEQ ID NO: 45 - DNA, 87 bp, Artificial Sequence
  Synthetic sequence
tctagaaataattttgtttaactttaagaaggagatatacatatggctagttccctaccccgtgatgcagagaatctgtactttcag

SEQ ID NO: 46 - DNA, 87 bp, Artificial Sequence
  Synthetic sequence
tctagaaataattttgtttaactttaagaaggagatatacatatggcttcctcccttccacgcgacgcagagaatttgtatttccag

SEQ ID NO: 47 - DNA, 87 bp, Artificial Sequence
  Synthetic sequence
tctagaaataattttgtttaactttaagaaggagatatacatatggcaagttcactccctcgggacgcagaaaatctgtactttcaa

SEQ ID NO: 48 - DNA, 87 bp, Artificial Sequence
  Synthetic sequence
tctagaaataattttgtttaactttaagaaggagatatacatatggccagttcgttgccccgtgatgctgagaatctgtacttccaa

SEQ ID NO: 49 - DNA, 87 bp, Artificial sequence
  Synthetic sequence
tctagaaataattttgtttaactttaagaaggagatatacatatggcttcgagtttaccacgtgacgctgagaatctgtacttccag

SEQ ID NO: 50 - DNA, 87 bp, Artificial Sequence
  Synthetic sequence
tctagaaataattttgtttaactttaagaaggagatatacatatggcctcgtctttaccccgtgatgcagagaatctgtattttcaa

SEQ ID NO: 51 - DNA, 87 bp, Artificial Sequence
  Synthetic sequence
tctagaaataattttgtttaactttaagaaggagatatacatatggcctcttcccttccgcgcgatgcagagaacttgtatttccaa

SEQ ID NO: 52 - DNA, 40 bp, Artificial Sequence
  Synthetic sequence
cgggaatccggtacccaagctaaacctcaaattccgaaag

SEQ ID NO: 53 - DNA, 45 bp, Artificial Sequence
  Synthetic sequence
ttttttccgctcgagtttgacttctgtagctacaaagattttacg

SEQ ID NO: 54 - DNA, 28 bp, Artificial Sequence
  Synthetic sequence
tatacatatggcttcgtcccaccatcac

SEQ ID NO: 55 - DNA, 31 bp, Artificial Sequence
  Synthetic sequence
tctccttcttaaagttaaacaaaattatttc

SEQ ID NO: 56 - DNA, 51 bp, Artificial Sequence
  Synthetic sequence
cgcggatcccatatggctagcagcgaaaacctgtattttcagggcagcacc

SEQ ID NO: 57 - DNA, 28 bp, Artificial Sequence
  Synthetic sequence
ggcgaattcggtacccggcagctgggtg

SEQ ID NO: 58 - DNA, 23 bp, Artificial Sequence
  Synthetic sequence
gagaatttgtacttccaaggatc

SEQ ID NO: 59 - DNA, 21 bp, Artificial Sequence
  Synthetic sequence
ggacgaagccatatgtatatc

SEQ ID NO: 60 - DNA, 24 bp, Artificial Sequence
  Synthetic sequence
catcattgagatccggctgctaac

SEQ ID NO: 61 - DNA, 24 bp, Artificial Sequence
  Synthetic sequence
atgatggtggtggtggtggtggtg

SEQ ID NO: 62 - DNA, 43 bp, Artificial Sequence
  Synthetic sequence
tgggtccggtggtagtggtgggagtcaagctaaaccgcagatc

SEQ ID NO: 63 - DNA, 44 bp, Artificial sequence
  Synthetic sequence
cctgaacctcccgatcccccggtaccaggcagttgtgtgttaag

SEQ ID NO: 64 - DNA, 44 bp, Artificial sequence
  Synthetic sequence
tgggtccggtggtagtggtgggagtcaagctaaacctcaaattc

SEQ ID NO: 65 - DNA, 44 bp, Artificial sequence; identical to SEQ ID NO: 63
  Synthetic sequence

SEQ ID NO: 66 - DNA, 18 bp, Artificial sequence
  Synthetic sequence
ttgggggaggagatgcgt

SEQ ID NO: 67 - DNA, 21 bp, Artificial sequence
  Synthetic sequence
gggttgtaccttagccttcac

SEQ ID NO: 68 - DNA, 18 bp, Artificial sequence
  Synthetic sequence
tatagtgatgagttgcgc

SEQ ID NO: 69 - DNA, 19 bp, Artificial sequence
  Synthetic sequence
gggttgtaccttagccttc

SEQ ID NO: 70 - DNA, 27 bp, Artificial sequence
  Synthetic sequence
cgatgccgagaatttgtacttccaagg

SEQ ID NO: 71 - DNA, 29 bp, Artificial sequence
  Synthetic sequence
cgtggcaaggacgaagccatatgtatatc

SEQ ID NO: 72 - DNA, 40 bp, Artificial sequence
  Synthetic sequence
cgcggaaaacctgtattttcagggatcgacgttttccaag

SEQ ID NO: 73 - DNA, 50 bp, Artificial sequence
  Synthetic sequence
tcacgaggtaaagaactggccatatgtatatctccttcttaaagttaaac

SEQ ID NO: 74 - DNA, 40 bp, Artificial sequence
  Synthetic sequence
cgcagagaatttgtatttccagggatcgacgttttccaag

SEQ ID NO: 75 - DNA, 50 bp, Artificial sequence
  Synthetic sequence
tcgcgtggaagggaggaagccatatgtatatctccttcttaaagttaaac

SEQ ID NO: 76 - DNA, 27 bp, Artificial sequence
  Synthetic sequence
gcgcagccaagctaaacctcaaattcc

SEQ ID NO: 77 - DNA, 26 bp, Artificial sequence
  Synthetic sequence
ggcaccagggtaccctctaaagctgc

SEQ ID NO: 78 - DNA, 32 bp, Artificial sequence
  Synthetic sequence
gcgctcccaagctaaacctcaaattccgaaag

SEQ ID NO: 79 - DNA, 26 bp, Artificial sequence
  Synthetic sequence
ggcacgagactcccaccactaccacc

SEQ ID NO: 80 - DNA, 20 bp, Artificial sequence
  Synthetic sequence
gaaaacctgtattttcaggg

SEQ ID NO: 81 - DNA, 21 bp, Artificial sequence
  Synthetic sequence
gctgctagccatatgtatatc

SEQ ID NO: 82 - DNA, 56 bp, Artificial sequence
  Synthetic sequence
aagaaggagatatacatatggccagttctgaaaacctgtattttcagggatcgacg

SEQ ID NO: 83 - DNA, 72 bp, Artificial sequence
  Synthetic sequence
caccagggtaccctctaaagctgcagcacctgtaccaggtaactgtgtatttaactttttagtatattcttc

SEQ ID NO: 84 - DNA, 18 bp, Artificial sequence
  Synthetic sequence
ctgcctggtaccggggga

SEQ ID NO: 85 - DNA, 25 bp, Artificial sequence
  Synthetic sequence
tccctgaaaatacaggttttccgcg

SEQ ID NO: 86 - DNA, 37 bp, Artificial sequence
  Synthetic sequence
gccgatttgctttccggatctgcctggtaccggggga

SEQ ID NO: 87 - DNA, 45 bp, Artificial sequence
  Synthetic sequence
ggaatgcttttggtgcagcgtccctgaaaatacaggttttccgcg

SEQ ID NO: 88 - DNA, 61 bp, Artificial sequence
  Synthetic sequence
cacctgcagctggccggtgtgcacccgcaacggcctgccggtgaccgggggatcgggaggt

SEQ ID NO: 89 - DNA, 69 bp, Artificial sequence
  Synthetic sequence
cagcccggggtgttgcaggtgccgcccacgcaggtttcgccgcatccctgaaaatacaggttttccgcg

SEQ ID NO: 90 - DNA, 37 bp, Artificial sequence
  Synthetic sequence
gccgatttgctttccggatctgcctggcacaggtgct

SEQ ID NO: 91 - DNA, 45 bp, Artificial sequence
  Synthetic sequence
ggaatgcttttggtgcagcgtccttggaagtacaaattctcggac

SEQ ID NO: 92 - DNA, 40 bp, Artificial sequence
  Synthetic sequence
tccgccgatttgctttccggatctgcctggcacaggtgct

SEQ ID NO: 93 - DNA, 48 bp, Artificial sequence
  Synthetic sequence
atgcttttggtgcagcggccacctccttggaagtacaaattctcggac

SEQ ID NO: 94 - DNA, 43 bp, Artificial sequence
  Synthetic sequence
tgggtccggtggtagtggtgggagtctggtgccgcgcagccaa

SEQ ID NO: 95 - DNA, 50 bp, Artificial sequence
  Synthetic sequence
cctgaacctcccgatcccccggtaccaggcagatccggaaagcaaatcgg

SEQ ID NO: 96 - DNA, 24 bp, Artificial sequence
  Synthetic sequence
ggtggctgcggcgaaacctgcgtg

SEQ ID NO: 97 - DNA, 26 bp, Artificial sequence
  Synthetic sequence
tccttggaagtacaaattctcggacg

SEQ ID NO: 98 - DNA, 40 bp, Artificial sequence
  Synthetic sequence
gtccggtggtagtggtgggagtctggtgccgcgcagccaa

SEQ ID NO: 99 - DNA, 42 bp, Artificial sequence
  Synthetic sequence
ccacctgaacctcccgatccccctgtcacaggcaggccgttg

SEQ ID NO: 100 - DNA, 43 bp, Artificial sequence
  Synthetic sequence
ctatgatcatccggaaatttgcggtctgcctggcacaggtgct

SEQ ID NO: 101 - DNA, 51 bp, Artificial sequence
  Synthetic sequence
ttgcagcgcggatcgctgcagcaacctccttggaagtacaaattctcggac

SEQ ID NO: 102 - DNA, 43 bp, Artificial sequence; identical to SEQ ID NO: 94
  Synthetic sequence

SEQ ID NO: 103 - DNA, 47 bp, Artificial sequence
  Synthetic sequence
cctgaacctcccgatcccccggtaccaggcagaccgcaaatttccgg

SEQ ID NO: 104 - protein, 6 aa, Artificial Sequence
  Synthetic sequence
  misc_feature (3)..(3): Xaa can be any naturally occurring amino acid
LPXTGA

SEQ ID NO: 105 - protein, 5 aa, Artificial sequence
  Synthetic sequence
  misc_feature (3)..(3): Xaa can be any naturally occurring amino acid
LAXTG

SEQ ID NO: 106 - protein, 5 aa, Artificial sequence
  Synthetic sequence
  misc_feature (3)..(3): Xaa can be any naturally occurring amino acid
LPXSG

SEQ ID NO: 107 - protein, 6 aa, Artificial sequence
  Synthetic sequence
LPGTGA

SEQ ID NO: 108 - protein, 6 aa, Artificial sequence
  Synthetic sequence
LPSTGA

SEQ ID NO: 109 - protein, 6 aa, Artificial sequence
  Synthetic sequence
LPETGA

SEQ ID NO: 110 - protein, 5 aa, Artificial sequence
  Synthetic sequence
LPGTG

SEQ ID NO: 111 - protein, 5 aa, Artificial sequence
  Synthetic sequence
LPGTA

SEQ ID NO: 112 - protein, 5 aa, Artificial sequence
  Synthetic sequence
LPSTG

SEQ ID NO: 113 - protein, 5 aa, Artificial sequence
  Synthetic sequence
LPSTA

SEQ ID NO: 114 - protein, 5 aa, Artificial sequence
  Synthetic sequence
LPETG

SEQ ID NO: 115 - protein, 5 aa, Artificial sequence
  Synthetic sequence
LPETA

SEQ ID NO: 116 - protein, 5 aa, Artificial sequence
  Synthetic sequence
LPATG

SEQ ID NO: 117 - protein, 5 aa, Artificial sequence
  Synthetic sequence
  misc_feature (3)..(3): Xaa can be any naturally occurring amino acid
LPXSG

SEQ ID NO: 118 - protein, 5 aa, Artificial sequence
  Synthetic sequence
  misc_feature (3)..(3): Xaa can be any naturally occurring amino acid
LPXTG

SEQ ID NO: 119 - protein, 5 aa, Artificial sequence
  Synthetic sequence
  misc_feature (3)..(3): Xaa can be any naturally occurring amino acid
LAXTG

SEQ ID NO: 120 - protein, 5 aa, Artificial sequence
  Synthetic sequence
  misc_feature (3)..(3): Xaa can be any naturally occurring amino acid
APXTG

SEQ ID NO: 121 - protein, 5 aa, Artificial Sequence
  Synthetic sequence
  misc_feature (3)..(3): Xaa can be any naturally occurring amino acid
FPXTG

SEQ ID NO: 122 - protein, 5 aa, Artificial Sequence
  Synthetic sequence
LMVGG

SEQ ID NO: 123 - protein, 5 aa, Artificial Sequence
  Synthetic sequence
  MISC_FEATURE (3)..(5): GGS triplet can be repeated at least one, two, three, four, or five times
GSGGS

SEQ ID NO: 124 - protein, 4 aa, Artificial Sequence
  Synthetic sequence
GAAA

SEQ ID NO: 125 - protein, 4 aa, Artificial Sequence
  Synthetic sequence
LEGT

SEQ ID NO: 126 - protein, 8 aa, Artificial Sequence
  Synthetic sequence
GAAALEGT

SEQ ID NO: 127 - protein, 14 aa, Artificial Sequence
  Synthetic sequence
GSGGSGGSGGSGGS

SEQ ID NO: 128 - protein, 12 aa, Artificial Sequence
  Synthetic sequence
LPGTGAAALEGT

SEQ ID NO: 129 - protein, 19 aa, Artificial Sequence
  Synthetic sequence
LPGTGGSGGSGGSGGSGGS

SEQ ID NO: 130 - protein, 5 aa, Artificial Sequence
  Synthetic sequence
LVPRS

SEQ ID NO: 131 - protein, 8 aa, Artificial Sequence
  Synthetic sequence
GGSGGSGG

SEQ ID NO: 132 - protein, 5 aa, Artificial Sequence
  Synthetic sequence
LPRDA

SEQ ID NO: 133 - protein, 8 aa, Artificial Sequence
  Synthetic sequence
ENLYFQGS

SEQ ID NO: 134 - protein, 7 aa, Artificial Sequence
  Synthetic sequence
ENLYFQG

SEQ ID NO: 135 - protein, 8 aa, Artificial Sequence
  Synthetic sequence
GAAALEGT

SEQ ID NO: 136 - protein, 14 aa, Artificial Sequence
  Synthetic sequence
GSGGSGGSGGSGGS

SEQ ID NO: 137 - protein, 10 aa, Artificial Sequence
  Synthetic sequence
HHHHHHHHHH

SEQ ID NO: 138 - protein, 6 aa, Artificial Sequence
  Synthetic sequence
HHHHHH

SEQ ID NO: 139 - protein, 4 aa, Artificial Sequence
  Synthetic sequence
QAKP

SEQ ID NO: 140 - DNA, 42 bp, Artificial Sequence
  Synthetic sequence
tctagaaataattttgtttaactttaagaaggagatatacat

SEQ ID NO: 141 - DNA, 45 bp, Artificial Sequence
  Synthetic sequence
atggcttcgtccttgccacgcgatgccgagaatttgtacttccaa

SEQ ID NO: 142 - DNA, 10 bp, Artificial Sequence
  Synthetic sequence
aagaaggaga